(19) United States
(12) Patent Application Publication
(10) Pub. No.: US 2025/0331884 A1
(43) Pub. Date: Oct. 30, 2025

(54) DEVICES AND METHODS FOR TISSUE REMOVAL AND WOUND CLOSURE

(71) Applicant: Osheru, Inc., Bend, OR (US)

(72) Inventors: Patricia O. Buehler, Bend, OR (US); Knute C. Buehler, Bend, OR (US); Matthew W. Hoskins, Bend, OR (US); Michael J. Allison, Ridgefield, WA (US); Michael A. Fairchild, Vancouver, WA (US); John Muri, Laguna Niguel, CA (US)

(21) Appl. No.: 19/193,799

(22) Filed: Apr. 29, 2025

Related U.S. Application Data
(60) Provisional application No. 63/640,843, filed on Apr. 30, 2024.

(51) Int. Cl. A61B 17/3205; A61B 90/00

(52) U.S. Cl. CPC A61B 17/3205; A61B 90/03; A61B 90/08; A61B 2090/034; A61B 2090/0807

(57) ABSTRACT

Disclosed herein are systems, devices, kits, and associated methods for excising tissue. A device for excising tissue may include a body, a first curved member coupled to the body and having a first tissue contact surface, a second curved member movable relative to the first curved member and comprising a second tissue contact surface, an actuator coupled to the body and configured to decrease a distance between the first and second curved members, and a cutting assembly configured to excise the tissue. The first tissue contact surface may have a first radius of curvature (ROC), while the second curved member may have a second, smaller ROC. Further, one or portions of the device, such as one or both of the first and second jaws, may be transparent and/or may include markings to facilitate accurate aligning of the device relative to a tissue excision site.

### CROSS REFERENCE TO RELATED APPLICATIONS

This application claims priority to U.S. Provisional Patent Application No. 63/640,843 filed Apr. 30, 2024, the content of which is incorporated herein by reference in its entirety for all purposes.

### TECHNICAL FIELD

Described herein are systems, devices, kits, and methods for excising tissue and/or sealing tissue excision wounds.

### BACKGROUND

Numerous diseases and conditions necessitate the removal of tissue from patients as part of diagnosis or treatment. One common example is blepharoplasty, a surgical procedure for removing excess tissue from the upper eyelids for medical or cosmetic purposes. Typically, this procedure involves several steps: marking the excision sites symmetrically for aesthetic outcomes, administering local anesthesia to the excision sites (which may cause tissue swelling), excising the excess tissue, and closing the wound caused by the excision. During tissue removal, cautery is often utilized to manage bleeding, while precise suturing is important to minimize scarring and seal the wound effectively. Thus, these procedures are often time-consuming and require skilled surgeons. Furthermore, post-operative care can be uncomfortable, risky, and prolonged for patients, with potential complications such as wound dehiscence, suture granulomas, and asymmetry between excision sites. Patients can also experience bruising and swelling around the excision sites for several weeks, and typically need to have a suture removal appointment during the recovery period.

Given these challenges, there is a need for advancements in tissue excision technology, including technology capable of both excess tissue removal and wound closure. Accordingly, new and improved systems, devices, kits, and methods for tissue removal and wound closure are described herein.

### BRIEF SUMMARY

Described herein are devices and methods for excising tissue and/or sealing wounds, such tissue and wounds of an upper eyelid of a patient.

A device for excising tissue may include a body, a first curved member coupled to the body and having a first tissue contact surface, a second curved member movable relative to the first curved member and having a second tissue contact surface, an actuator coupled to the body and configured to decrease a distance between the first and second curved members, and a cutting assembly configured to excise the tissue. The first tissue contact surface may have a first radius of curvature (ROC), and the second tissue contact surface may have a second, smaller ROC. In some variations, the first ROC may be about 30 mm to about 40 mm and the second ROC may be about 25 mm to about 35 mm. Similarly, the first ROC may be about 1 to about 1.5 times greater than the second ROC. In some variations, when the first and second curved members are in a closed configuration, a gap may be formed between proximal and distal ends of the first and second tissue contact surfaces. In some variations, the second curved member may further include a first projection coupled to a first end of the second curved member and a second projection coupled to a second, opposite end of the second curved member. The first curved member may include a first channel at least partially surrounding first projection and a second channel at least partially surrounding the second projection, and the first and second projections may be configured to be translated vertically within the first and second channels, respectively, in response to actuation of the actuator. In some variations, the second curved member may further include a tissue support surface adjacent to the second tissue contact surface. The tissue support surface may be raised relative to the second tissue contact surface along a longitudinal axis of the device. The tissue support surface and the second tissue contact surface may form a slot therebetween, and the slot may be configured to receive a portion of a cutting element of the cutting assembly therein during excision of the tissue. In some variations, the first curved member may be fixed to the body. The first curved member may be integrally formed with the body. In some variations, the first and second tissue contact surfaces may face one another. In some variations, one or both of the first and second tissue contact surfaces may be serrated. In some variations, one or both of the first or second tissue contact surfaces may be smooth. In some variations, the first tissue contact surface may include a male engagement feature and the second tissue contact surface may include a female engagement feature configured to receive the male engagement feature therein. The male engagement feature may be a tongue and the female engagement feature may be a groove, and the male and female engagement features may extend along a length of the first and second tissue contact surfaces, respectively. Each of the male and female engagement features may include a triangular cross-sectional shape, and a height of the tongue and a depth of the groove may each be about 0.025 mm to about 2 mm. In some variations, the actuator may include a lever. In some variations, the cutting assembly may be coupled to and moveable along the first curved member. The cutting assembly may be coupled to and moveable along a cutting guide of the first curved member. In some variations, the cutting assembly may include a housing having a handle and a cutting element coupled to the housing. The device may further include a cutting assembly lock coupled to one or both of the first and second curved members, where the cutting assembly lock may be configured to prevent movement of the cutting element after the cutting assembly is moved from a first position to a second position along the first curved member. The first position may be an initial position of the cutting assembly prior to tissue excision and the second position may be a final position of the cutting assembly after tissue excision. Moreover, the cutting element may be a blade. In some variations, the device may include a cutting guide having a channel formed along a top surface of the first jaw. The body may include first and second grip members extending from opposing lateral sides of the body. In some variations, the device may be configured to be used on an upper eyelid, a lower eyelid, an eyeball, an eyebrow, a cheek, a jaw, an underarm, or a neck of a patient. The device may be configured to be used on only an upper right eyelid or only an upper left eyelid of the patient. The device may be configured to be used on a conjunctiva of the eyeball of the patient. In some variations, the tissue may include lesion on the patient.

Another device for excising tissue may include a body having a first jaw with a first tissue contact surface and a cutting guide, where the cutting guide includes a first engagement feature, a second jaw movable relative to the first jaw and having a second tissue contact surface, an actuator coupled to the body and configured to move one of the first or second jaws, and a cutting assembly slidably coupled to the cutting guide and configured to excise the tissue. The cutting assembly may include a second engagement feature configured to engage the first engagement feature to limit movement of the cutting assembly along the cutting guide. The first engagement feature may include a recess and the second engagement feature may include a hook. In some variations, the first jaw may further include at least one detent configured to engage the cutting assembly and limit movement of the cutting assembly along the cutting guide. The at least one detent may include a first detent configured to engage the cutting assembly when the cutting assembly is in a first position along the cutting guide and a second detent configured to engage the cutting assembly when the cutting assembly is in a second, different position along the cutting guide. The cutting assembly may include a first housing portion, a second housing portion, and a cutting element. The cutting element may be secured between the first and second housing portions. The first housing portion may include a channel configured to receive the second housing portion therein, and the housing portions may be coupled to one another with a coupling element such as a screw or pin. The first housing portion may include a contact surface having at least one curved region, and the curved region may be configured to receive user contact to move the cutting assembly along the cutting guide. The second housing portion may include a slot configured to receive the cutting element therein.

Another device for excising tissue may include a body, a first jaw having a first tissue contact surface, a linkage assembly, a second jaw movable relative to the first jaw and having a second tissue contact surface, an actuator having a first engagement feature and a first end coupled to the second jaw, a linkage arm having a first end coupled to the actuator and a second end coupled to the body, a biasing member anchored to the body and configured to bias the actuator toward an open position, a lock coupled to the body and having a second engagement feature configured to releasably couple with the first engagement feature to maintain a clamped positioned of the second jaw, and a cutting assembly configured to excise the tissue. The actuator may be configured to pivot relative to the body to move the second jaw linearly relative to the first jaw. The lock may be configured to be pivoted to disengage the second engagement feature from the first engagement feature. The first engagement feature may include a recess and the second engagement feature may include a hook. In some variations, the biasing member may be a tension spring. The actuator may include a bent lever. The body may define an opening that houses the lock therein. The device may further include a baseplate with an integrated stop member having a contact surface configured to engage an underside of the second jaw to limit movement of the second jaw away from the first jaw. The baseplate may include at least one coupling element configured to secure the baseplate to the body. In some variations, the actuator may further include an integrated stop member and a contact surface, where the stop member is configured to maintain the contact surface against an underside of the second jaw to limit movement of the second jaw away from the first jaw. The stop member may include an angled protrusion on an underside of the first end of the actuator. In some variations, the linkage arm may include an integrated stop member configured to contact an underside of the actuator to limit rotation of the actuator. The stop member may include an angled protrusion on an underside of the first end of the linkage arm. The actuator may include a contact surface that is maintained against the second jaw by the stop member of the linkage arm.

Another device for excising tissue may include a body, a first jaw coupled to the body and having a first tissue contact surface and one or more markings configured to aid in alignment of the device with a tissue excision site on a patient, a second jaw movable relative to the first jaw and having a second tissue contact surface, an actuator coupled to the body configured to move one of the first and second jaws relative to the other of the first and second jaws, a lock coupled to the body and configured to fix a position of the one of the first and second jaws relative to the other of the first and second jaws when engaged, and a cutting assembly configured to excise the tissue. The first and second jaws may be transparent. In some variations, the one or more markings may include at least one marking centered along a longitudinal axis of the first jaw. The one or more markings may be on a top surface of the first jaw. Further, the first jaw may include a cutting guide configured to support the cutting assembly, and the one or more markings may be on a top surface of the cutting guide. In some variations, the one or more markings may be one or more first markings, and the second jaw may further include one or more second markings. In some variations, one or both of the first and second tissue contact surfaces may be serrated. In some variations, the lock may include a first engagement feature configured to releasably couple to a second engagement feature of the actuator. The first engagement feature may be a first projection extending from a body of the lock, and the second engagement feature may be a second projection extending transversely to the first projection and from the actuator, and the second projection include an opening configured to receive the first projection therein. The lock may further include a lock actuator configured to remove the first engagement feature from the second engagement feature. Additionally, the lock actuator may be configured to receive an applied force that is greater than a compression force within the lock to remove the first engagement feature from the second engagement feature. The compression force may be applied by a spring of the lock, and the lock actuator may be configured to apply an opposite force to the spring to remove the first engagement feature from the second engagement feature. In some variations, the lock may include an engageable latch. In some variations, the body may further include a handle configured to be held by a user. In some variations, the actuator may be rotatably coupled to the second jaw via one or more pivot joints, which may include at least two pivot joints.

Another device for excising tissue may include a first jaw moveable relative to a second jaw, a cutting assembly coupled to and moveable along the first jaw, and an actuator configured to move the first jaw relative to the second jaw. The cutting assembly may include a housing with a handle and a cutting element coupled to the housing and having a cutting edge facing toward a first end of the housing, where the handle may be positioned on the first end of the housing. In some variations, the first jaw may include a cutting guide configured to support the cutting assembly. The cutting guide may include a first track configured to support a first portion of the housing of the cutting assembly and a second track configured to support a second portion of the housing. The first track may have a first thickness that is less than a second thickness of the second track. Further, the first and second tracks may have first and second radii of curvature (ROC) that are about equal to an ROC of the first jaw. In some variations, the handle may extend from a top surface of the housing of the cutting assembly, or may extend from a first side surface of the housing of the cutting assembly. The cutting element may be coupled to and extend from a second side surface of the housing. Additionally, the first and second sides may be opposite sides of the housing. In some variations, a central longitudinal axis of the handle of the cutting assembly and a longitudinal axis of the cutting element of the cutting assembly may be parallel to each other. In some variations, the cutting edge of the cutting element may be angled relative to a central longitudinal axis of the cutting assembly. The cutting edge may have an angle of about 30 degrees relative to the central longitudinal axis. In some variations, the cutting element may include a double bevel. In some variations, the housing may further include a contact surface configured to support a finger of a user thereon.

Yet another device for excising tissue may include a body, a first jaw coupled to the body, a second jaw movable relative to the first jaw, an actuator coupled to the body configured to move the second jaw relative to the first jaw;, a lock coupled to the body and configured to fix a position of the second jaw relative to the first jaw when engaged with the actuator, and a cutting assembly configured to excise the tissue. The lock may include a releasable ratchet.

A kit for excising tissue may include a first device for excising tissue and a second device for excising tissue. The first device may include a first body, a first jaw coupled to the first body, a second jaw movable relative to the first jaw, a first cutting assembly coupled to and moveable along the first jaw, and a first actuator coupled to the first body. The first cutting assembly may include a first housing having a first handle, and a first cutting element coupled to the first housing and having a first cutting edge. The first actuator may be configured to move the second jaw relative to the first jaw. The second device may include a second body, a third jaw coupled to the second body, a fourth jaw movable relative to the third jaw, a second cutting assembly coupled to and moveable along the third jaw, and a second actuator coupled to the second body. The second cutting assembly may include a second housing having a second handle and a second cutting element coupled to the second housing and having a second cutting edge. The second actuator may be configured to move the fourth jaw relative to the third jaw. Additionally, the first and second cutting edges may be facing opposite directions. In some variations, the first device for excising tissue may be configured to be used on a first tissue excision site on a first eyelid of a patient, and the second device for excising tissue may be configured to be used on a second tissue excision site on a second, different eyelid of the patient. In some variations, the first and second devices may be configured to be used simultaneously during a procedure for excising tissue. In some variations, the kit may further include a forceps configured to position the tissue between one or both of the first and second jaws and the third and fourth jaws of the first and second devices, respectively. Additionally, or alternatively, in some variations, the kit may further include one or more of a local anesthetic and an adhesive. The local anesthetic may be lidocaine, and the adhesive may be cyanoacrylate.

A method for excising tissue may include aligning a device for excising tissue relative to a tissue excision site on a patient through a transparent first jaw of the device using one or more markings on the first jaw, positioning the tissue through the first jaw and a second jaw of the device, where the first jaw is coupled to a body of the device and the second jaw is movable relative to the first jaw, closing the first and second jaws using an actuator of the device and excising the tissue. In some variations, positioning the tissue may include grasping the tissue and positioning the tissue through an opening between the first and second jaws. In some variations, the method may further include, prior to excising the tissue, releasably engaging the actuator with a lock of the device to maintain the first and second jaws in a closed configuration. In some variations, the tissue may be excised using a cutting assembly of the device. The cutting assembly may be configured to be moved from a first position to a second position along the first jaw of the device to excise the tissue. Further, the cutting assembly may include a housing having a handle and a cutting element coupled to the housing, and wherein moving the cutting assembly comprises sliding the cutting assembly along the first jaw using the handle. In some variations, the method may further include removing the excised tissue. In some variations, a wound caused by excising the tissue may be sealed during one or more of closing the first and second jaws and excising the tissue. The wound may be sealed without using sutures or cautery. Further, the method may further include applying adhesive to the wound. In some variations, the method may further include, prior to aligning the device relative to the tissue excision site, applying a local anesthetic to the tissue excision site.

Another method for excising tissue may include, with a first device for excising tissue: aligning the first device relative to a first tissue excision site on a patient, positioning first tissue between a first jaw and a second jaw of the first device, actuating a first actuator of the first device to move the second jaw relative to the first jaw, and excising the first tissue using a cutting assembly of the first device. Simultaneously the method for excising tissue may include, with a second device for excising tissue: aligning the second device relative to a second tissue excision site on the patient, positioning second tissue between a third jaw and a fourth jaw of the second device, actuating a second actuator of the second device to move the fourth jaw relative to the third jaw, and excising the second tissue using a second cutting assembly of the second device. In some variations, the first tissue excision site may be on a first upper eyelid of the patient and the second tissue excision site may be on a second upper eyelid of the patient. In some variations, the first cutting assembly of the first device may include a first housing having a first handle positioned on a first end of the first housing and a first cutting element coupled to the housing and comprising a first cutting edge facing a first direction. Additionally, the second cutting assembly of the second device may include a second housing having a second handle positioned on a second end of the housing and a second cutting element coupled to the second housing and comprising a second cutting edge facing a second, opposite direction. In some variations, the method may further include aligning the first device relative to the second device.

### BRIEF DESCRIPTION OF THE DRAWINGS

FIG. 1A depicts a perspective view of a variation of a first tissue excision device. FIG. 1B depicts a perspective view of a variation of a second tissue excision device.

FIG. 2 depicts an exploded view of a variation of a tissue excision device.

FIG. 3A depicts a front view of a variation of a body of a tissue excision device. FIG. 3B is a back view of the body of FIG. 3A.

FIG. 4A depicts a back view of a variation of a tissue excision device. FIG. 4B depicts a top view of another variation of a tissue excision device.

FIG. 5 depicts a cross-sectional view of a variation of jaws of a tissue excision device.

FIG. 6A depicts a top view of a variation of a tissue excision device. FIG. 6B depicts a front view of the tissue excision device of FIG. 6A.

FIG. 7 depicts a magnified view of a variation of jaws of a tissue excision device.

FIG. 8 depicts a magnified view of a variation of serrations on jaws of a tissue excision device.

FIG. 9 depicts a perspective view of a portion of a variation of a tissue excision device.

FIG. 10A depicts a top view of a variation of a tissue excision device showing the jaws thereof. FIG. 10B is a perspective view of a variation of a second jaw of the jaws of FIG. 10A.

FIG. 11A depicts an exploded view of a variation of an actuator of a tissue device. FIG. 11B depicts a bottom view of a variation of a tissue excision device including the actuator of FIG. 11A.

FIG. 12 depicts an exploded view of a variation of a lock of a tissue excision device.

FIG. 13 depicts a perspective view of a portion of a variation of a body of a tissue excision device having an actuator and a lock coupled thereto.

FIG. 14 depicts a cross-sectional view of a variation of a tissue excision device in a closed configuration.

FIG. 15 depicts a front view of a variation of a cutting assembly of a tissue excision device.

FIG. 16A depicts a top view of a variation of a housing of a cutting assembly of a tissue excision device. FIG. 16B depicts a top perspective view of the housing of FIG. 16A. FIG. 16C depicts a bottom perspective view of the housing of FIGS. 16A-16B. FIG. 16D depicts a first side view of the housing of FIGS. 16A-16C. FIG. 16E depicts a second side view of the housing of FIGS. 16A-16D. FIG. 16F depicts a third side view of the housing of FIGS. 16A-16E.

FIG. 17A depicts a top view of another variation of a housing of a cutting assembly of a tissue excision device. FIG. 17B depicts another top view of the housing of FIG. 17A. FIG. 17C depicts a bottom view of the housing of FIGS. 17A-17B. FIG. 17D depicts a first side view of the housing of FIGS. 17A-17C. FIG. 17E depicts a second side view of the housing of FIGS. 17A-17D. FIG. 17F depicts a third side view of the housing of FIGS. 17A-17E.

FIG. 18 depicts a front view of a variation of a cutting element of a cutting assembly of a tissue excision device.

FIG. 19 depicts a front view of another variation of a cutting element of a cutting assembly of a tissue excision device.

FIGS. 20A and 20B depict front and exploded views, respectively, of another variation of a tissue excision device. FIGS. 20C and 20D depict front views of the tissue excision device of FIGS. 20A-20B in an open configuration and a closed configuration, respectively.

FIGS. 21A-21C depict front perspective, exploded, and back views, respectively, of another variation of a tissue excision device. FIGS. 21D and 21E depict front perspective views of the tissue excision device of FIGS. 21A-21C in an open configuration and a closed configuration, respectively.

FIGS. 22A and 22B depict front and exploded views, respectively, of yet another variation of a tissue excision device. FIGS. 22C and 22D depict front views of the tissue excision device of FIGS. 22A-22B in an open configuration and a closed configuration, respectively.

FIGS. 23A and 23B depict front and exploded views, respectively, of still another variation of a tissue excision device. FIGS. 23C and 23D depict front views of the tissue excision device of FIGS. 23A-23B in an open configuration and a closed configuration, respectively.

FIGS. 24A and 24B depict first and second side views, respectively, of a variation of graspers.

FIG. 25 is a flow diagram illustrating a variation of a method for excising tissue using a tissue excision device.

FIG. 26 is a flow diagram illustrating another variation of a method for excising tissue using first and second tissue excision devices.

FIGS. 27A and 27B depict front and side views, respectively, of an exemplary configuration of a wound caused by excision of excess tissue of an upper eyelid.

FIGS. 28A and 28B depict a perspective view of another variation of a tissue excision device in open and closed configurations, respectively.

FIG. 29 depicts an exploded view of the variation of the tissue excision device shown in FIGS. 28A and 28B.

FIG. 30 depicts a back view of another variation of a tissue excision device.

FIGS. 31A and 31B depict a cross-sectional view of a variation of a tissue excision device comprising a tongue-in-groove clamping mechanism in partially open and closed configurations, respectively.

FIGS. 32A and 32B depict a perspective view and a top view of another variation of a tissue excision device, respectively.

FIG. 33 depicts a front view of another variation of a tissue excision device.

FIG. 34 depicts another front view of the variation of the tissue excision device shown in FIG. 33.

FIG. 35 depicts a cross-sectional side view of a variation of a tissue excision device comprising a linkage assembly.

FIG. 36 depicts a perspective view of another variation of a tissue excision device.

FIGS. 37A and 37B depict a cross-sectional perspective view and a side view of a variation of a tissue excision device with a first jaw stop mechanism in partially open and fully open configurations, respectively.

FIGS. 38A and 38B depict a perspective view and a side view of a variation of a tissue excision device with a second jaw stop mechanism, respectively.

FIGS. 39A and 39B depict an exploded view and an assembled bottom view of a variation of a cutting assembly for a tissue excision device, respectively.

FIG. 40A depicts a side view of another variation of a cutting assembly for a tissue excision device. FIG. 40B depicts a side view of a portion of a tissue excision device comprising the cutting assembly of FIG. 40A.

FIG. 41A depicts a perspective view of a variation of a guillotine-type tissue excision device. FIG. 41B depicts a perspective view of a first jaw of the tissue excision device of FIG. 41A. FIG. 41C depicts a perspective view showing the integration of the first jaw with a body of the tissue excision device of FIG. 41A. FIG. 41D depicts a perspective view of a second jaw of the tissue excision device of FIG. 41A. FIG. 41E depicts a perspective view of a cutting element of the tissue excision device of FIG. 41A.

### DETAILED DESCRIPTION

The following description, for purposes of explanation, uses specific nomenclature to provide a thorough understanding of the invention. The foregoing descriptions of specific variations of the invention are presented for purposes of illustration and description. They are not intended to be exhaustive or to limit the invention to the precise forms disclosed. Indeed, it should be understood that modifications and variations to the devices and methods herein may be possible in view of the teachings disclosed herein.

Described herein are systems, devices, kits, and methods for manipulating, positioning, grabbing, clamping, excising, and/or removing tissue of one or more tissue excision sites of a patient. Additionally, or alternatively, the systems, devices, kits, and methods herein may be used for wound closure, such as for sealing a wound of a tissue excision site created during a tissue excision procedure.

As referred to herein throughout, a tissue excision site may include a portion (e.g., at least a portion) of tissue in or on an anatomical area or feature of a patient, such as, for example, tissue of: an upper eyelid, a lower eyelid, an eyeball (e.g., a conjunctiva), an eyebrow, a jaw, an underarm, a cheek, or a neck of the patient. The tissue may include, for example, skin (e.g., epidermis, dermis), such as skin lacerations, loose skin and/or skin lesions. Additionally, or alternatively, the tissue may include other lesions, such as lesions on the eyeball, subcutaneous tissue and/or fat. In some variations, a tissue excision site may include any suitable anatomical area having loose skin and/or a skin lesion to be excised.

As an example, excision of upper eyelid tissue, referred to in the art as blepharoplasty, may be performed in accordance with the methods herein, using variations of the devices described herein, for cosmetic and/or functional purposes (e.g., to improve peripheral vision for patients whose upper eyelids obscure their field of vision). However, conventional blepharoplasty may be costly, time-consuming, and risky for patients. For example, blepharoplasty is generally performed under local anesthesia and/or intravenous or oral sedation, requires an experienced surgeon, and takes about 1-2 hours to perform in a surgical operating room (an elevated cost comparable to the outpatient setting). For example, one time-consuming step of the procedure may include creating symmetric markings on both upper eyelids of the patient. In particular, good cosmetic and functional results of the procedure are dependent on the location of the incision and removal of the proper amount of excess skin tissue. Thus, symmetry between the upper eyelids is desirable to achieve a good cosmetic and functional result, hence a surgeon generally attempts to create an incision at a same location relative to each upper eyelid and attempts to remove a similar amount of tissue from each upper eyelid. Another time-consuming, and invasive, step of conventional blepharoplasty incudes controlling bleeding during the excisions using cautery to maintain a view of the markings guiding the surgeon during the excisions. Additionally, yet another time-consuming and invasive step of traditional blepharoplasty includes meticulous suturing of the excision wounds to reduce scarring thereof and to properly seal the wounds.

Furthermore, post-operative care may be time-consuming and difficult for patients. For example, common complications of blepharoplasty include wound dehiscence-separation of wound edges due to improper wound healing-and asymmetry-a difference in the amount of tissue excised and/or an improper alignment of the excisions of a patient's upper eyelids. Additionally, bruising and swelling of the eye area caused by invasive surgical techniques can take 2-3 weeks to heal. Moreover, patients often need to have a suture removal appointment during the recovery period, which may cause more discomfort and increase costs for the patient.

While the aforementioned disadvantages of tissue excision are described with respect to blepharoplasty, medical professionals and patients may experience some or all of the same disadvantages for other tissue excision procedures, such as skin lesion or loose skin removals.

Advantageously, the systems, devices, kits, and methods herein may benefit patients and medical providers by reducing costs, procedure time, complications, and recovery time for tissue excision procedures. For example, the tissue excision devices herein may systems, devices, kits, and methods herein may enable a surgeon or other medical professional to complete the procedure in about 10 minutes in an outpatient setting, which may significantly reduce costs for patients. Additionally, the systems, devices, kits, and methods herein may provide a minimally invasive alternative to traditional procedures by eliminating the need for local anesthesia injection, cautery and sutures. For example, the tissue excision devices herein may be configured to apply a clamping force to tissue to be excised that results in hemostasis of the tissue. The resulting hemostasis may eliminate a need for cautery to control bleeding during the procedure and may promoting self-sealing of a wound caused by the tissue excision by reducing a fluid concentration within the wound, thus increasing a cohesive force of the wound and allowing the vulnerable tissue to “stick” together. As a result, patients may experience less bruising and swelling, and faster recovery periods, than the current standard of care. In the same vein, eliminating a need for using sutures for wound closure may provide a better cosmetic result for patients by reducing scarring of the tissue excision site(s). Additionally, eliminating the need for cautery and/or suture may result in a simpler procedure for a surgeon or other medical professional to perform, as one or more steps of tissue excision may be eliminated. Further, the systems, devices, kits, and methods herein may provide consistent, symmetric results and a reduced risk of wound dehiscence compared to conventional procedures such as blepharoplasty. For example, the tissue excision devices herein may allow for adjustment of an amount of tissue to be removed, reducing asymmetry complications. Additionally, or alternatively, the tissue excision devices herein may include one or more features, such as transparent components and/or fiducial markings, to facilitate accurate alignment of the tissue excision devices with marked tissue excision sites.

Thus, in general, the technology herein may facilitate and accelerate tissue excision procedures for surgeons (or other medical professionals), which may be offered at lower cost compared to conventional tissue excision procedures. Further, the technology herein and may be less invasive and promote faster healing for the patient, thus addressing some of the deficiencies of current tissue excision procedures involving excess skin removal and wound closure.

Generally, the systems herein may be for performing a tissue excision procedure involving one or more tissue excisions. The systems may include one or more tissue excision devices and/or one or more graspers (e.g., forceps). The tissue excision devices may be configured to be aligned with a tissue excision site, such as with surgical markings made on the tissue excision site. For example, one or portions of a tissue excision device, such as the body and/or the jaws thereof, may be transparent and/or may include markings to facilitate accurate aligning of the device relative to a tissue excision site. The graspers may be used to position tissue within an opening (e.g., between jaws) of the tissue excision devices. The tissue excision devices may be configured to clamp the tissue within the device (e.g., within jaws thereof) at a force to cause hemostasis of the tissue, and may subsequently be used to excise the tissue. In some variations, the tissue excision devices herein may promote self-sealing of a wound caused by the excision due to the lack of blood flow to the tissue during the tissue excision. In some variations, the graspers herein may additionally be used to move excised tissue away from the tissue excision site. Variations tissue excision devices, graspers, and aspects thereof will be described in detail below.

The following description is exemplary in nature and is not intended to limit the scope, applicability, or configuration of the invention in any way. Various changes to the described embodiments may be made in the function and arrangement of the elements described herein without departing from the scope of the invention.

### 1. Devices

### 1.1. Tissue Excision Devices

The tissue excision devices herein may generally include a body, jaws, and an actuator, configured to move one or both of the jaws between an open configuration and closed configuration. In some variations, the devices may additionally include a lock and a cutting assembly. The body may include a handle configured to be held by a user (e.g., a surgeon or other medical professional) and may thus have any size, shape and form suitable to be held in a hand during a procedure. The jaws (which may be referred to herein as “curved members” or “elongate members”) may include, for example, a first jaw and a second jaw. The first and second jaws may be coupled, such as via a rotational and/or translational coupling. Additionally, the first and second jaws may be actuated between an open configuration and a closed configuration. The open configuration may allow for tissue to be positioned (e.g., using a grasper) within an opening defined by the first and second jaws, and the closed configuration may allow for tissue to be captured within the first and second jaws. Additionally, transparent portions (e.g., jaws and body) of the tissue excision devices, as well as markings thereon, may expand a user's view while positioning the tissue between the jaws and aligning the jaws symmetrically on a tissue excision site. In general, in the closed configuration, blood flow may be partially or completely restricted to tissue captured or clamped within the first and second jaws due to a clamping force and/or surface pattern (e.g., serration pattern) of the jaws. A uniform clamping force may be applied to the tissue due to a difference in the radii of curvature between the first and second jaws, and/or due to serrations along the first and second jaws.

Moreover, the actuator may be coupled to one or both of the first and second jaws, and may be configured to move one or both of the first and second jaws relative to each other to adjust a size of the opening formed therebetween. The lock may be configured to releasably engage the actuator so that a position of the jaws (e.g., configured by the actuator) may be fixed when the lock is engaged and one or both of the jaws may be free to move relative to the other of the jaws when the lock is, and disengaged or released. Further, the cutting assembly may be used to cut the excess tissue clamped within the jaws (e.g., tissue that is not held directly between the jaws, but is pushed in front of the jaws due to the clamping) to excise tissue. The cutting assembly may be integrated with the tissue excision device or may be a separate device. In some variations, the cutting assembly may be moveably coupled to the jaws (e.g., to one or both of the first and second jaws) and may be configured to excise tissue held within the jaws. For example, the cutting assembly may be configured to be contacted or grasped by one or more fingers of a user and moved along a portion of the jaws (e.g., following a contour thereof) to excise tissue positioned in front of a front side or external surface of the tissue excision device (e.g., a side or surface facing away from a patient or tissue excision site that is pushed in front of the jaws due to the clamping). In general, a wound created by the excision may partially or fully (e.g., at least partially) self-seal due to cohesive forces within the wound that are augmented by the lack of blood flow to the tissue excision site (e.g., caused by the clamping force and/or serration of the jaws). Put another way, the wound may “stick” to itself due to the lack of blood flow to the tissue excision site. Each of the body, jaws, actuator, lock, and cutting assembly will be described in detail herein.

In some variations, the tissue excision devices herein may be configured for use on one or more anatomical areas of a patient. For example, the tissue excision devices may be used to excise tissue on one or more of: an upper eyelid, a lower eyelid, an eyeball (e.g., a conjunctiva), an eyebrow, a jaw, an underarm, a cheek, and a neck of a patient. As another example, the tissue excision devices may be used on one or more lesions (e.g., skin lesions, eyeball lesions) located on any anatomical areas of a patient, including, without limitation, those listed above. In some variations, a geometry of the tissue excision devices herein may vary according to the intended anatomical area. For example, a first tissue excision device configured to be used on an eyelid or eyeball may have curvature, such as at least one curve matching an average contour of a human eyeball, while a second tissue excision device configured to be used on a jaw may not have a curvature, or may have less curvature (e.g., a greater radius of curvature) than the first tissue excision device. In some variations, one or more dimensions (e.g., length, width, depth) of the tissue excision devices herein may vary according to individualized measurements of the anatomic area based on imaging modalities and/or direct measurement. In some variations, one or more dimensions (e.g., length, width, depth) of the tissue excision devices herein may vary according to the intended anatomical area. For example, a first tissue excision device configured to be used on an underarm may have one or more of a greater length, width, and depth than a second tissue excision device configured to be used on a conjunctiva. In some variations, a tissue excision device may have one or more dimensions or geometry (e.g., length, width, depth, curvature) that are about equal to one or more corresponding dimensions of its intended anatomical area for treatment. For example, a tissue excision device configured for use on an underarm, jaw, or eyeball (e.g., conjunctiva) may have an average length and/or width that is about equal to an average length and/or width of an average adult underarm, jaw, or eyeball. Additionally, or alternatively, in some variations, a configuration of the tissue excision devices herein may vary depending on the intended anatomical area. For example, a first tissue excision device configured to be used on a first upper eyelid may include a cutting assembly configured to be moved in a first direction, while a second tissue excision device configured to be used on a second, opposite upper eyelid may include a cutting assembly configured to be moved in a second, opposite direction (as explained in detail herein).

Similarly, the tissue excision devices herein may be individually designed for each patient. For example, scanning technology (e.g., a 3D scanner) may be utilized to create a model (e.g., a 3D model) of a patient's tissue excision site. This data may be used to design a size and/or shape of a desired excision for the patient and to fabricate (e.g., 3D print, or the like) the patient specific device, including patient specific jaws (e.g., a first jaw, including a cutting guide thereon, and/or a second jaw) to achieve the desired excision.

As referred to herein throughout, a width of a tissue excision device may be defined as a distance along a transverse axis (e.g., a horizontal, abscissa, or X-axis) of the device between a first end (e.g., a first lateral end) and a second end (e.g., a second, opposite lateral end) of the device. In some variations, the first and second ends may be right and left ends of the tissue excision device according to a perspective of a user when the device is in-use. Moreover, a depth of the tissue excision device may be defined as a distance along a sagittal axis of the device between a third end (e.g., a front side) and a fourth end (e.g., a back side) of the device. In some variations, the third and fourth ends may be front and back sides of the tissue excision device according to the perspective of the user when the device is in-use. That is, the front side of a tissue excision device may be configured to face away from a patient and tissue excision site thereon while the device is in-use, and the back side may be configured to face toward (and in some variations, contact) the patient and the tissue excision site when the device is in-use. Further, a length of the tissue excision device may be defined as a distance along a longitudinal axis of the device between a fifth end (e.g., a bottom end or distal end) and a sixth end (e.g., a top end or proximal end) of the device. In some variations, the fifth and sixth ends may be distal and proximal ends of the tissue excision device according to the perspective of the user when the device is in-use.

In one example, a tissue excision device configured to be used on an upper eyelid may have a maximum width of about 20 mm to about 90 mm, such as about 50 mm to about 70 mm or about 60 to about 65 mm (e.g., about equal to or greater than 60 mm, about equal to or greater than 65 mm), a maximum depth of about 5 mm to about 50 mm, such as about 7.5 mm to about 20 mm or about 10 mm to about 15 mm (e.g., about equal to or greater than 10 mm), and a maximum length of about 30 mm to about 100 mm, such as about 60 mm to about 80 mm or about 70 mm to about 75 mm (e.g., about equal to or greater than 65 mm, about equal to or greater than 70 mm, about equal to or greater than 75 mm).

Moreover, one or more components of the tissue excision devices herein may be manufactured from a medical grade plastic and/or metal, such as stainless steel. In some variations, one or more components of the tissue excision devices herein, such as the jaws and/or the body, may be partially or fully composed of a transparent or translucent material. When being positioned against a tissue excision site, a partially or completely transparent body and/or jaws of a tissue excision device may provide a view of the tissue excision site through the device (e.g., through the jaws) to assist with accurate alignment of the device with the tissue excision site. Additionally, or alternatively, in some variations, one or more components of the tissue excision devices herein such as the jaws and/or the body, may include markings to guide alignment of the device with a tissue excision site. The markings may include etches, projections, indentations, colored markings, frosted markings, or combinations thereof. Additionally, the markings may be any suitable shape, such as a line, a circle, a triangle, a square, a rectangle, an irregular shape, or combinations thereof. In some variations, the markings may include at least one marking on the jaws (e.g., a first jaw, such as a superior or upper jaw) positioned on a central longitudinal axis of a tissue excision device to aid a user in aligning the device centrally on a tissue excision site. In some variations, the markings may include a plurality of markings, such as two, three, four, five, six, seven, eight, nine, ten, or more than ten markings on the jaws (e.g., a first jaw, such as a superior or upper jaw) to aid in alignment of the device relative to the tissue excision site.

In one example, tissue excision devices having transparent portions and/or markings to aid in alignment may additionally improve symmetry of a tissue excision procedure involving excisions on a plurality (e.g., two or more) of tissue excision sites. For example, as described herein throughout, the tissue excision devices herein may be used to perform a blepharoplasty procedure. Typically, a blepharoplasty procedure involves excising excess tissue from both upper eyelids of a patient. Accordingly, tissue excision devices having transparent portions and/or markings to guide alignment of the devices relative to tissue excision sites may improve the symmetry of the excisions made on the upper eyelids of the patient. That is, a first tissue excision device with a transparent portion and/or markings may allow a user to accurately align the first device on a first upper eyelid of the patient, and a second tissue excision device a same transparent portion and/or same markings may allow the user to accurately align the second device on a second upper eyelid of the patient. Thus, the excisions made with the first and second tissue excision devices on the first and second eyelids may be relatively precise and symmetric.

In some variations, the tissue excision devices herein may include one or more surface patterns, such as one or more labels and/or one or more logos. The surface patterns may be located on surfaces of the tissue excision devices herein. For example, a logo, such as a logo resembling an eye, may be included on (e.g., etched into or printed onto) a housing and/or actuator of a tissue excision device, such as into or onto a front surface (e.g., surface configured to face away from a tissue excision site and toward an external environment during a tissue excision procedure) of the housing and/or actuator. Additionally, or alternatively, a label, such as a label indicating an intended anatomical area to treat with a given tissue excision device may be included on (e.g., etched into or printed onto) a cutting assembly of the device, such as into or onto a top surface of the cutting assembly. The label may include text, such as a letter, a word, or more than one word, and/or may include an image, such as a symbol. In some variations, the label may include the letter “L” or the word “left” to indicate that a cutting assembly of a tissue excision device should be moved from right to left, according to a user's perspective of the tissue excision device when the device is in-use. Correspondingly, in some variations, the label may include the letter “R” or the word “right” to indicate that a cutting assembly of a tissue excision device should be moved from left to right according to a user's perspective of the tissue excision device when the device is in-use.

In some variations, the tissue excision devices herein may be disposable. For example, a tissue excision device may be configured for a single use to excise a single portion of tissue. In some variations, a single-use tissue excision device may additionally be configured to enable a single cut using the device. For example, tissue held within the tissue excision device may be cut as a cutting assembly of the device is moved from a first (e.g., initial or pre-cutting) position to a second (e.g., final or post-cutting) position along the tissue excision device (e.g., along a first jaw thereof). In some variations, the tissue excision device may additionally include a cutting assembly lock configured to prevent movement of the cutting assembly (e.g., of a cutting element of the cutting assembly) after the cutting assembly has been moved from the first position to the second position. Moreover, in some variations, the tissue excision devices herein may be multi-use devices configured to excise one or more tissues and/or to enable one or more cuts of tissue with a cutting assembly configured to be transferred between the first and second positions along the device any suitable number of times.

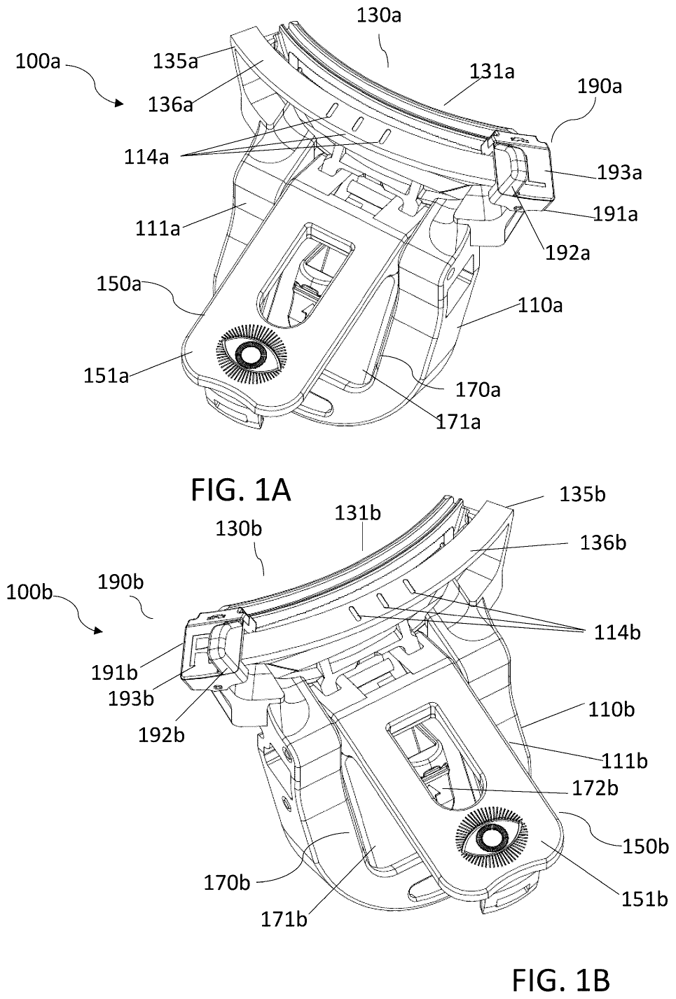

FIGS. 1A and 1B are perspective views of exemplary tissue excision devices (“devices”) 100a,b. As shown, both devices 100a,b may include bodies 110a,b, jaws 130a,b, actuators 150a,b, locks 170a,b, and cutting assemblies 190a,b. The devices 100a,b may have an intended alignment relative to a tissue excision site. For example, first sides 111a,b of the devices 100a,b may be front sides configured to face away from the tissue excision site, toward an external environment, so that a user may access the actuators 150a,b, locks 170a,b, and cutting assemblies 190a,b during a tissue excision procedure. Oppositely, second sides (not shown) of the devices 100A,B may be back sides configured to face toward the tissue excision site to align with (e.g., be in at least partial contact with) site during the tissue excision procedure. The bodies 110a,b may include handles 113a,b configured to be held in a first hand, or between a first set of figures, of a user. In some variations, the handles 113a,b may be transparent. Similarly, the jaws 130a,b, which may each include first jaws 131a,b and second, opposing jaws (not shown), may be transparent to allow a user to view a tissue excision site through the jaws 130a,b. The first jaws 131a,b may include top surfaces 136a,b having markings 114a,b to aid alignment of the jaws 130a,b against a tissue excision site.

The top surfaces 136a,b may be top surfaces of cutting guides 135a,b of each of the first jaws 131a,b. The cutting guides 135a,b may be curved, elongate supports configured to carry cutting assemblies 190a,b thereon, respectively. The cutting assemblies 190a,b may be moved along the cutting guides 135a,b to excise tissue, as is explained in detail herein.

Moreover, the cutting assemblies 190a,b, which may be configured to excise tissue by cutting the tissue, may have unique configurations. As one example, a housing 191a of the cutting assembly 190a may include a handle 192a projecting from a first end (e.g., a left end from a perspective of a user of the device 100a) of the housing 191a, and a contact surface 193a on a second, opposite end (e.g., a right end from a perspective of a user of the device 100a) of the housing 191a. In contrast, a housing 191b of the cutting assembly 190 may include a handle 192b projecting from a first end (e.g., a right end from a perspective of a user of the device 100b) of the housing 191b, and a contact surface 193b on a second, opposite end (e.g., a left end from a perspective of a user of the device 100b) of the housing 191a. The handles 192a,b may be configured to be grasped or pressed by a user to move (e.g., slide) the cutting assemblies 190a,b along the first jaws 131a,b. The contact surfaces 193a,b may be configured to receive a finger of a user thereon to facilitate user controllability of movement of the cutting assemblies 190a,b.

Further, the devices 100a,b may include actuators 150a,b, which may be configured to actuate (e.g., move) the jaws 130a,b between open and closed configurations. The actuators 150a,b may have actuator bodies 151a,b, which may be levers and may be configured to actuate the first jaws 131a,b and/or the second jaws relative to each other. The actuators 150a,b may be configured to engage locks 170a,b to maintain a closed configuration of the jaws 130a,b (e.g., to clamp tissue therebetween). Additionally, the locks 170a,b may have lock bodies 171a,b including lock actuators 172a,b configured to release the actuators 150a,b, from the locks 170a,b so that the jaws 130a,b may be moved from the closed configuration to the open configuration (e.g., after the devices 100a,b are used to excise tissue).

In practice, to excise tissue using either one of the devices 100a,b, the first jaw 131a,b and the second jaw 132a,b may be in an open configuration while the cutting assembly 190a,b may be positioned in an initial or pre-cutting position at a first or second lateral end of the cutting guide 135a,b. Excess tissue may be positioned between first and second jaws 131a,b and 132a,b. Using pre-drawn markings as a guide, the tissue may be positioned such that the surgical markings are aligned with the markings 114a,b of the jaws 130a,b. Then, the actuator 150a,b may be moved (e.g., pivoted) in a first direction to move the second jaw 132a,b closer to the first jaw 131a,b so that tissue may be clamped therebetween. Additionally, the actuator 150a,b may be engaged with the lock 170a,b to maintain a closed configuration where the tissue is clamped within the jaws 130a,b. Next, using the handle 192a,b, the cutting assembly 190a,b may be pushed in a first or second direction (toward a lateral end that is opposite to the lateral end where the cutting assembly 190a,b is in the initial position), following a curvature along the first side 111a,b of the device 100a,b, to excise the tissue that sits on the front side of the jaws 130a,b. The tissue may be excess tissue protruding from between the jaws 130a,b, and toward a surgeon or other medical professional using the device 100a,b. Next, the actuator 150a,b may be moved in a second, opposite direction, away from the first side 111a,b, to open the jaws 130a,b, and the device 100a,b may be removed from the surgical field to prepare for wound closure (e.g., using a tissue adhesive).

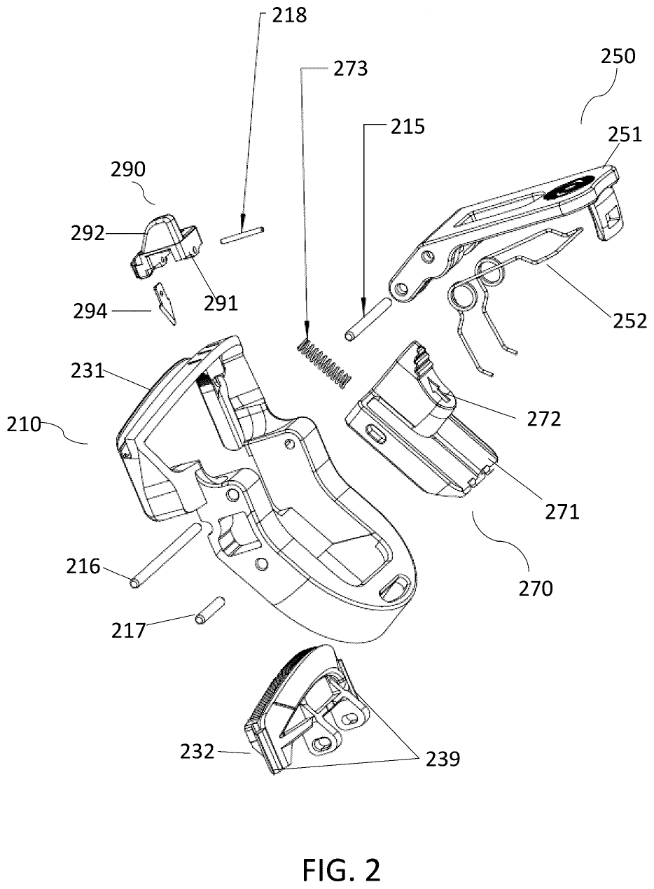

Turning to FIG. 2, shown there is an exploded view of an exemplary tissue excising device (“device”) 200 showing each component of the device 200 and illustrating how each component of the device 200 may be coupled to the body 210 thereof. As shown, a first jaw 231 of the device 200 may be integrally formed with the body 210. In contrast, a second jaw 232 of the device 200 may be moveably coupled to the body 210 via projections 239 that are configured to move (e.g., translate vertically) within channels (not shown) of first jaw 231. In some variations, however, the first jaw 231 may similarly moveably coupled to the second jaw 232, which may be fixed to (e.g., integrally formed with) the body 210. Additionally, the second jaw 232 may be coupled to the actuator 250 via a first retention pin 215. The actuator 250 may have an actuator body 251 and may be coupled to the body 210 via a pivot pin 216. Accordingly, the second jaw 232 may be indirectly coupled to body 210 via the actuator 250. Further, the actuator body 251 may be configured to receive a first spring 252 (e.g., within one or more recesses of a back side of the actuator body), such as, for example, a torsion spring, which may be coupled to the pivot pin 216 (e.g., coiled around the pivot pin 216 at one or more pivot joints, such as two pivot joints). The first spring 252 may additionally be coupled to a lock body 271 of a lock 270 (e.g., within one or more recesses of a back side of the lock body 271). The lock 270 may be coupled to the body 210 via one or more second retention pins 217 (e.g., one or two second retention pins 217). The lock body 271 may additionally be configured to receive a second spring 273, such as, for example, a compression spring, which may be housed within a recess (not shown) of a back side of the lock body 271. The second spring 273 may be configured to produce a force within the lock 270 to form an engagement between the lock 270 and the actuator 250. For example, the spring may push the lock body 271 into or onto a component of the actuator body 251 to force an engagement between the lock 270 and actuator body 250. The lock 270 body may also include lock actuator 272, which may be configured to receive an applied force that is greater than the compression force produced by the second spring 273 to release the engagement between the lock 270 and the actuator 250. Moreover, a housing 291 of the cutting assembly 290 may be coupled to the first jaw 231 via a third retention pin 218. The housing 291 may comprise a handle 292 extending therefrom, which may be configured to be contacted (e.g., grasped, pushed) by a user to move the cutting assembly 290 along the first jaw 231. Additionally, a cutting element 294 of the cutting assembly may be coupled to the housing 291 (e.g., to an interior surface thereof) via the third retention pin 218.

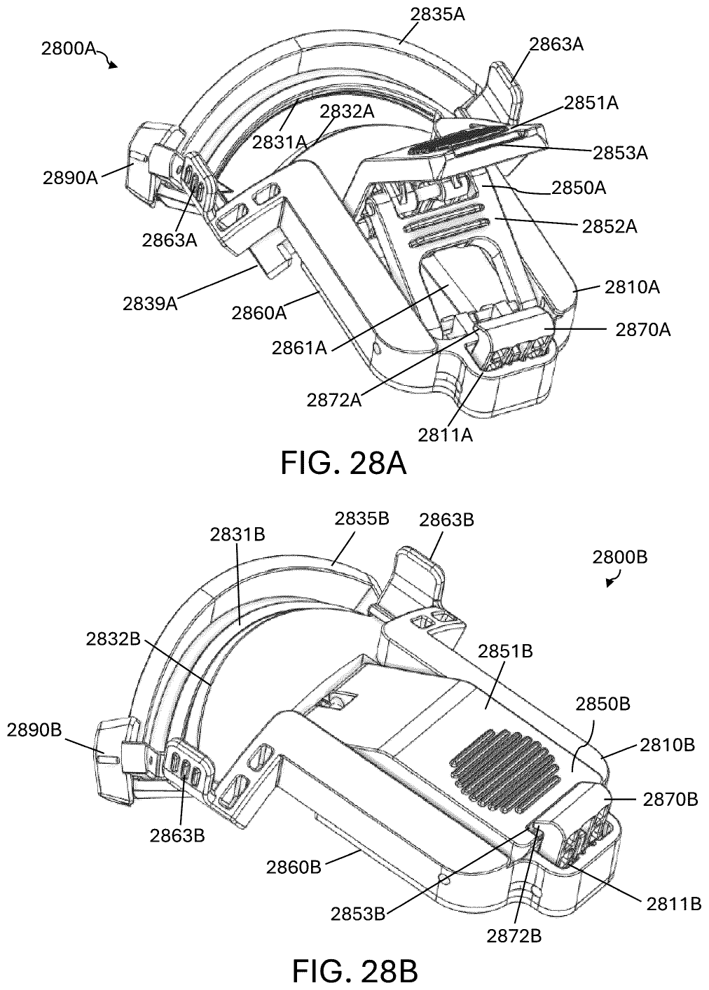

Another exemplary configuration of a tissue excision device is shown in FIGS. 28A and 28B. These figures show perspective views of the tissue excision device 2800A/B in open and closed configurations, respectively. The device 2800A/B may have a body 2810A/B which may comprise a frame defining an internal cavity for housing various components, such as a linkage assembly 2850A/B. The body 2810A/B may also define a separate opening 2811A/B for housing one or more additional components, such as a lock 2870A/B (which may also be part of the linkage assembly 2850A/B). The body may comprise a distal (top, upper) portion defining a first jaw 2831A/B and lateral ends coupled thereto, and a proximal (bottom, lower) portion defining the internal cavity housing the linkage assembly 2850A/B. In some variations, a maximum width of the distal portion may be greater than a maximum width of the proximal portion. As depicted in FIGS. 28A and 28B, in some variations, lateral sidewalls of the proximal portion of the body 2810A/B may be substantially linear and may taper, but need not.

The first jaw 2831A/B may be fixed relative to the body 2810A/B and may comprise a first tissue contact surface (not shown) and a cutting assembly guide 2835A/B formed along a surface of the first jaw 2831A/B opposite the first tissue contact surface. In some variations, the first jaw 2831A/B may be integrally formed with the body 2810A/B. Further, a second jaw 2832A/B may be movably coupled to the body 2810A/B and may comprise a second tissue contact surface (not shown). One or both of the first and second jaws 2831A/B, 2831A/B may be configured to move relative to the other and/or to the body 2810A/B. For example, the second jaw 2832A/B may be configured to move relative to the first jaw 2831A/B to clamp tissue positioned between the jaws.

The cutting assembly guide 2835A/B may be configured to receive a cutting assembly 2890A/B thereon. In some variations, the first jaw 2831A/B may comprise one or more surface features (e.g., detents and/or protrusions-not shown) configured to engage the cutting assembly 2890A/B in one or more discrete positions along the cutting guide 2835A/B, such as at the beginning and/or an end of a cutting path. For example, the surface feature(s) may be configured to maintain a position of the cutting element of the cutting assembly 2890A/B prior to use of the device 2800A/B (e.g., during shipping). Accordingly, in some variations, the surface detents and/or protrusions may be positioned at a first lateral end of the cutting guide 2835A/B and/or at a second, opposite end of the cutting guide 2835 A/B.

The linkage assembly 2850A/B may comprise components that are movably (e.g., pivotably) coupled to one or both of the first and second jaws 2831A/B, 2832A/B and/or to the body 2810A/B in order to actuate the device 2810A/B for clamping and releasing tissue. For example, the linkage assembly 2850A/B may comprise actuator 2851A/B, a linkage arm 2852A (shown in FIG. 28A), and a biasing member 2861A (shown in FIG. 28A). The actuator 2851A/B may have a first end rotatably coupled to the second jaw 2832A/B. In some variations, the actuator 2851A/B may comprise a lever configured to rotate (e.g., pivot) relative to the body 2810A/B to cause linear movement of the second jaw 2832A/B. In some variations, the lever may be bent or may otherwise comprise a curve along its longitudinal axis. The linkage arm 2852A may have a first end coupled to the actuator 2851A/B (e.g., adjacent to or at the bend or apex of the curve) and a second end coupled to the body 2810A/B. The biasing member 2861A may comprise a tension spring having a first end coupled to the actuator 2851A/B or linkage arm 2852A and a second end coupled to the body 2810A/B. The biasing member 2861A may be configured to bias the actuator 2851A/B toward an open position in which the second jaw 2832A/B is spaced apart from the first jaw 2831A/B.

The actuator 2851 may further comprise a first engagement feature, such as a recess or projection, and the lock 2870A/B may comprise a second engagement feature, such as a complementary projection or recess. The lock 2870A/B may be positioned within the opening 2811A/B of the body 2810A/B and may be moveable to engage or disengage the first engagement feature of the actuator 2851. For example, the lock 2870A/B may be rotatable (e.g., pivotable) and/or translatable to engage or disengage the first engagement feature of the actuator 2851. When engaged, the lock 2870A/B may releasably couple with the actuator 2851A/B to maintain a closed or clamped position of the second jaw 2832A/B.

In some variations, the body 2018A/B may further comprise one or more components to facilitate gripping/grasping of the device 2800A/B, such as a pair of grip members 2863A/B extending from opposite lateral sides of the body 2810. The grip members 2863A/B may be shaped to facilitate handling, and may be textured and/or contoured to improve tactile feedback and control.

Furthermore, a baseplate 2860A/B may be coupled to a portion of the body 2810A/B, such as to an underside of a lower or proximal portion of the body 2810A/B. In some variations, the baseplate 2860A/B may comprise an integrated stop member (not shown) configured to limit movement of the second jaw 2832A/B away from the first jaw. As discussed in more detail herein, this stop member may thus define a fully open position of the jaws.

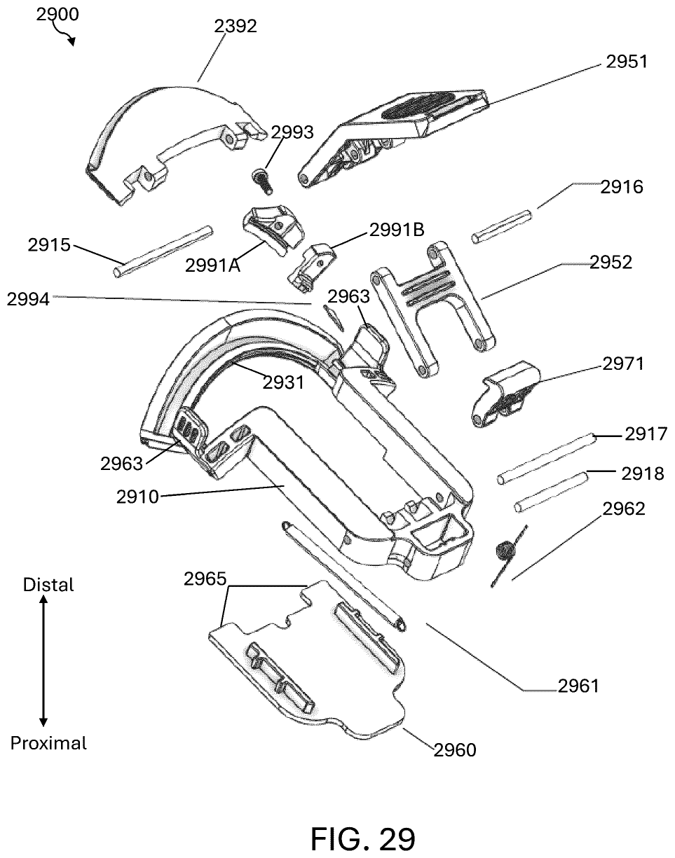

FIG. 29 depicts an exploded view of a tissue excision device 2900, which may correspond generally to the device shown in FIGS. 28A and 28B. The device 2900 may comprise a body 2910, a first jaw 2931 (which may be integrally formed with the body 2910), and a second jaw 2932. The second jaw 2932 may be movable relative to the body 2910 and first jaw 2931. The device 2900 may be configured to permanently or temporarily couple with a cutting assembly 2990, which may include a first housing portion 2991A, a second housing portion 2991B, and a cutting element 2994. The first and second housing portions 2991A, 2991B may be coupled to one another to hold the cutting element 2994 in a secure position. In some variations, a coupling element 2993, such as a screw or pin, may be used to hold the housing portions together. In other variations, the housing portions may be joined via a snap fit, interlocking features, adhesive, or another mechanical fastening technique.

The first housing portion 2991A may define at least one channel configured to couple to at least a portion of a cutting assembly guide (e.g., to a sidewall of a track thereof) on the first jaw 2931. In some variations, the first housing portion 2991A may comprise a contact surface having one or more contoured regions, such as curved or raised portions. These contoured regions may be configured to receive user contact and facilitate manual actuation of the cutting assembly 2990 along the cutting guide during use. The second housing portion 2991B may define a slot or groove for receiving the cutting element 2994, which may be secured therein via a pin or other suitable coupling feature. The second housing portion 2991B may be releasably coupled with the first housing portion 2991A, such as, for example, via a slot thereof that is sized and shaped to receive the second housing portion 2991B.

As described with respect to FIG. 28 above, a linkage assembly may be positioned within the body 2910 and may include an actuator 2951, a pivot arm or linkage arm 2952, a biasing member 2961, a lock 2971, and the second jaw 2931. As shown, dowels or pins 2915, 2916, 2917, 2918 may be used to couple ends of the second jaw 2932, biasing member 2961, and linkage arm 2952 to portions of the actuator body 2951 and the body 2910. The biasing member 2961 may comprise a spring, such as a tension spring, having a first end secured to the actuator 2951 and a second end secured to the body 2910.

The actuator 2951 may be configured to rotate (e.g., pivot) relative to the body 2910 to move the second jaw 2932 linearly relative to the first jaw 2931. In some variations, the actuator 2951 may include an integrated stop member configured to contact the second jaw 2932 to limit distal movement of the second jaw 2932 away from the first jaw 2931. Similarly, in some variations, a stop member may be provided on (or as part of) a baseplate 2960 that may be secured to the body 2910. The stop member may comprise a fixed projection configured to engage an underside of the second jaw 2932 to limit separation between the jaws. In some variations, the projection may be angled. In yet another variation, a stop member may be integrated into the linkage arm 2952 and positioned to contact a underside surface of the actuator 2951 to restrict rotational freedom of the actuator and maintain the actuator in contact with the second jaw 2932. For example, the stop member may comprise an angled protrusion formed on a first end (e.g., upper region) of the linkage arm 2952.

The lock 2971 may be actuatable (e.g., pivotable, translatable) via an internal biasing member 2962, which may comprise a spring (e.g., a torsion spring, a tension spring, etc.). The lock 2971 may be provided within an opening or compartment of the body 2910 that structurally limits rotational/translational movement of the lock 2972.

Moreover, a pair of grip members 2963 may extend laterally from opposite sides of the body 2910. These grip members may be extensions which may comprise recesses, ridges, and/or other surface texture to enhance usability.

Features and functions of several components of the tissue excision devices herein, including the body, the jaws, the linkage assembly (e.g., the actuator, the lock) and the cutting assembly, are described independently in more detail below.

### Body

The tissue excision devices herein may generally include a body (e.g., base, handle). The body may comprise a frame forming a distal (upper, top) jaw portion and a proximal (lower, bottom) handle portion. The handle portion may be configured to be held in a hand of a user, such as a surgeon or other medical professional. Additionally, as described above with respect to FIG. 2, the body may directly or indirectly (e.g., via pins) couple to other components of the tissue excision devices. For example, the body may include one or more lumens therethrough, which may be used to house pins to couple the body to other components of the tissue excision device. In some variations, the body may be integrally formed with one or more other components of the tissue excision device, such as with a first portion of the jaws (e.g., with a first jaw thereof). In some variations, at least a portion of the body may be constructed of a transparent or translucent material to facilitate visualization of a tissue excision site through the body.

In some variations, one or more maximum dimensions of the body may define one or more maximum dimensions of a tissue excision device. For example, a tissue excision device and its body may have a maximum width of about 20 mm to about 90 mm, such as about 50 mm to about 70 mm or about 60 mm to about 65 mm (e.g., about equal to or greater than 60 mm, about equal to or greater than 65 mm), a maximum depth of about 5 mm to about 50 mm, such as about 7.5 mm to about 20 mm or about 10 mm to about 15 mm (e.g., about equal to or greater than 10 mm), and a maximum length of about 30 mm to about 100 mm, such as about 60 mm to about 80 mm or about 70 mm to about 75 mm (e.g., about equal to or greater than 65 mm, about equal to or greater than 70 mm, about equal to or greater than 75 mm).

A width of the body may be defined as a distance along a transverse axis (e.g., a horizontal or X-axis) of the device between a first end (e.g., a first lateral end) and a second end (e.g., a second, opposite lateral end) of the device. Moreover, a depth of the body may be defined as a distance along a sagittal axis (e.g., Z-axis) of the device between a third end (e.g., a front side) and a fourth end (e.g., a back side) of the body. Further, a length of the body may be defined as a distance along a longitudinal axis (e.g., vertical or Y-axis) of the device between a fifth end (e.g., a bottom end or distal end) and a sixth end (e.g., a top end or proximal end) of the device. In some variations, the fifth and sixth ends may be distal and proximal ends of the tissue excision device according to the perspective of the user when the device is in use. In some variations, a maximum width of a distal (jaw-side) portion of the body may be greater than a maximum width of a proximal (handle-side) portion of the body.

In general, the body may be fabricated of a rigid or semirigid material, such as metal or polymer (e.g., polycarbonate, polypropylene).

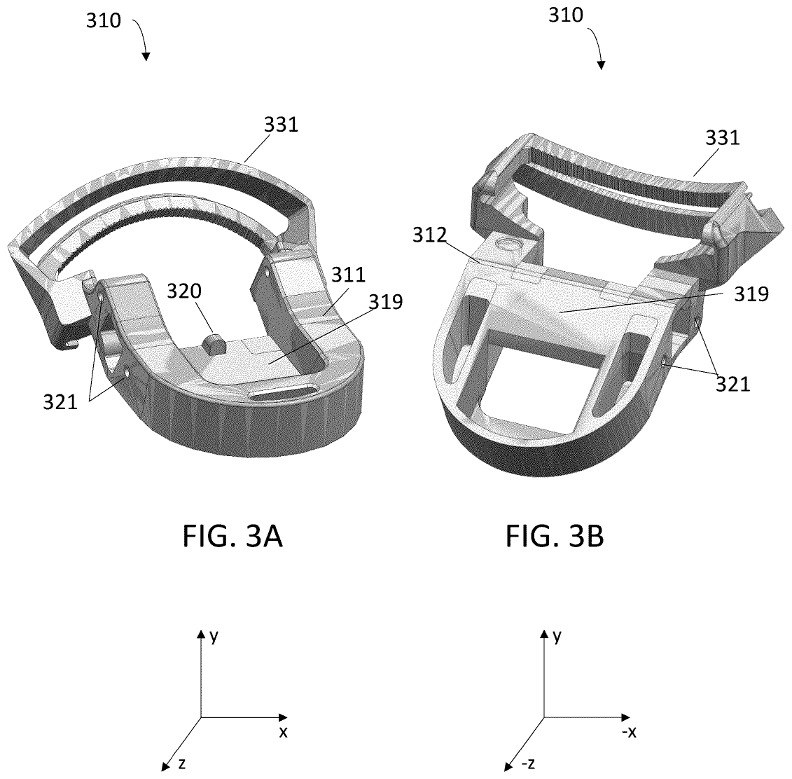

A front view of an exemplary body 310, including a first side 311 of the body 310, which may be a front side thereof, is shown in FIG. 3A. The front side 311 may be configured to face away from a tissue excision site during tissue excision. A back view of the body 310 of FIG. 3A, including second side 312 of the body 310, which may be a back side (underside), is shown in FIG. 3B. The back side 312 may be configured to face toward, and in some variations, partially contact, a tissue excision site during tissue excision. Referring to both FIGS. 3A and 3B, the body 310 may be integrally formed with the first jaw 331. Moreover, the body 310 may include a center support 319 configured to stabilize the structure of the body 310. Additionally, or alternatively, the center support 319 may be configured to hold a lock spring (e.g., spring 273 of FIG. 2) within a recess of a lock (e.g., lock 270 of FIG. 2) of the device by preventing the lock spring from moving out of the recess. As depicted in FIG. 3A, the center support 319 may include a projection 320 extending from a front side of the center support 319. The projection 320 may be configured to hold the lock spring in place by maintaining constant contact with a first end of the lock spring. Further, a lock actuator of the lock (e.g., lock actuator 272 of FIG. 2) may be configured to compress the lock spring against the projection 320 to release an engagement between the lock and an actuator of the device (e.g., actuator 250 of FIG. 2), as will be described in further detail herein.

While the body 310 is shown to have a contoured profile, the tissue excision devices herein may not require such a shape. In some variations, the body may comprise a substantially linear or tapered profile along one or more sides or edges. The body may further comprise one or more openings or compartments to at least partially enclose various components, such as a linkage assembly, actuator, and/or lock. Additionally, the body may comprise coupling features, such as lumens, channels, or engagement recesses, configured to receive or secure a movable jaw and/or a baseplate. In some variations, the baseplate may be coupled to the body using one or more fasteners, snap-fit features, or integrated joining elements.

The body may also comprise one or more grip members, which may be integrally formed with or separately attached to the body and may be contoured, textured, and/or ergonomically shaped to improve tactile engagement with the user. In some variations, the grip members may comprise arcuate outer profiles or recessed areas to facilitate thumb or other finger placement during use. In some variations, the body may comprise a pair of grip members extending from opposing lateral sides of a distal portion of the body.

Additionally, one or more guide channels may be formed within the body. These guide channels may be configured to receive and constrain lateral coupling features of the second jaw. In some variations, the second jaw may include projections or tabs that extend laterally into the guide channels, allowing the second jaw to translate along a linear axis relative to the body. Put another way, the second jaw may be linearly movable within guide channels formed in the distal portion of the body and constrained against rotational or transverse displacement during actuation. In this manner, the guide channels may facilitate controlled and repeatable movement of the second jaw toward and away from the first jaw to provide a consistent clamping motion.

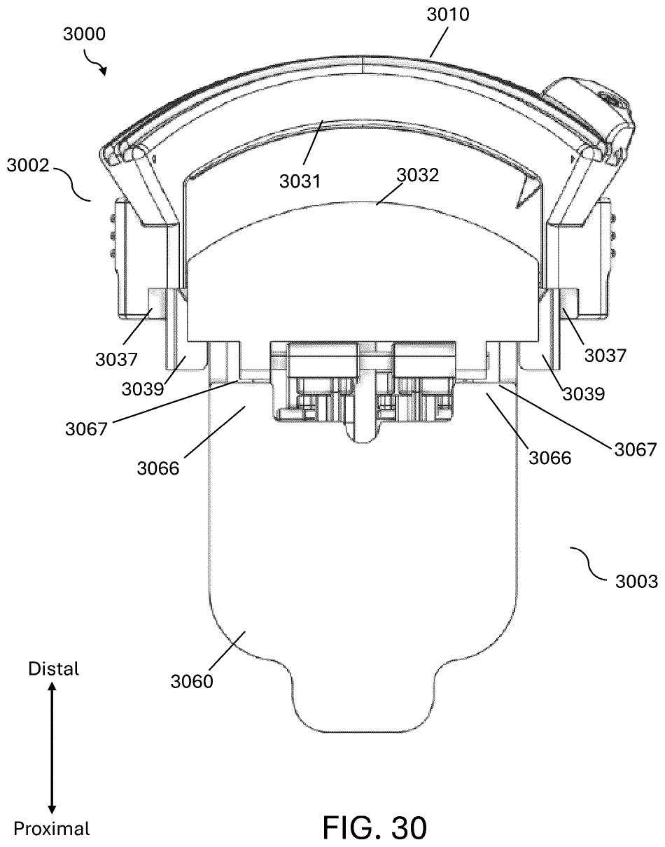

Referring to FIG. 30, a back view of an exemplary tissue excision device 3000 is shown. The device 3000 may comprise a body 3010 having a proximal portion 3003 and a distal portion 3002. The proximal portion 3003 may form a handle portion of the device 3000, and the distal portion 3002 may form or support a jaw portion of the device 3000. In some variations, the distal portion 3002 may be integrally formed with a first jaw 3031. A second jaw 3032 may be coupled to the body 3010 and linearly movable relative to the body 310/the first jaw 3031.

A baseplate 3060 may be coupled to the back/underside of the body 3010. The baseplate 3060 may comprise an integrated stop member 3066. The stop member 3066 may define or include a contact surface 3067 that is configured to engage a proximal or underside portion of the second jaw 3032 to limit movement of the second jaw away from the first jaw 3031. The stop member 3066 may be formed as an angled or raised projection, tab, rail, or pad. In some variations, the stop member 3066 may be integral with the baseplate 3060 or separately formed and secured to the baseplate. In other variations, the stop member may be provided on a different component of the device, such as on the actuator or linkage arm, and positioned to produce a similar limiting effect on jaw separation (described in further detail below).

Lateral portions of the body 3010 may further include coupling guides 3037. These coupling guides 3037 may be recessed channels, tracks, or lumens formed along internal surfaces of the body and may be configured to receive corresponding lateral coupling features 3039 of the second jaw 3032. When the coupling features 3039 are positioned within the coupling guides 3037, linear travel of the second jaw 3032 is enabled and rotational movement is constrained, ensuring smooth and directed clamping motion. The guides and features may comprise corresponding geometries to facilitate secure but slidable engagement between the jaws and body.

The body 3010 may also comprise one or more grip members or finger rest structures (not shown) that may protrude from opposing lateral sides of the distal portion to improve ergonomic control and user comfort during use.

### Jaws

The tissue excision devices herein may comprise jaws, such as a first jaw and a second jaw (which may also be referred to as “first curved member” and “second curved member” or “first elongate member” and “second elongate member”). The first and second jaws may be elongate members operably coupled together so that one or both may be moved (relative to one another) between an open configuration and a closed configuration. These configurations may be defined by a dimension (e.g., a length) of an opening between a first tissue contact surface of the first jaw and a second tissue contact surface of the second jaw. The first and second tissue contact surfaces may be configured to directly contact tissue during tissue excision, and may face (e.g., oppose) one another so that when tissue is positioned between the first and second jaws, one or both of the first and second tissue contact surfaces may be moved toward one another (e.g., via an actuator) to capture and secure the tissue between the first and second tissue contact surfaces. In some variations, at least a portion of one or both of the first and second jaws may be constructed of a transparent or translucent material to facilitate visualization of a tissue excision site through the jaws.

In some variations, the jaws may further comprise one or more tissue engagement features configured to enhance tissue securement, alignment, and/or cutting precision. These engagement features may comprise complementary tongue-and-groove configurations, serrations, textured surfaces, or other mechanical interface elements that promote positive mechanical coupling between the jaws when in the closed configuration. Such engagement features may serve multiple functions, including but not limited to: improving grip on tissue, promoting hemostasis along compression lines, maintaining precise jaw alignment during actuation, and defining consistent tissue compression profiles across the jaw length.

In some variations, the jaws may be biased in the open configuration by a biasing member (e.g., a spring), such as via a biasing member within a lock configured to engage with an actuator of the jaws (as explained in more detail herein). In the open configuration, the first and second jaws (e.g., the first and second tissue contact surfaces thereof) may have an opening with a length that is greater than a length of the opening in the closed configuration. The opening may be positioned over a tissue excision site, and tissue to be excised from the site may be pulled through the opening (e.g., using a grasper). One or both of the first and second jaws may be moved (e.g., via an actuator) to decrease a size of the opening (e.g., in one or more dimensions, such as a vertical dimension) and transition to the closed configuration, which may allow the first and second jaws to clamp the tissue therebetween. In some variations, the movement of the jaws may include linear or translational movement, such as vertical translational movement along a longitudinal axis of a tissue excision device. A first dimension of the opening (e.g., length, or vertical distance between the first and second tissue contact surfaces) may be variable within about 0 mm to about 20 mm, such as about 1 mm to about 18 mm, about 2 mm to about 16 mm, about 3 mm to about 14 mm, about 4 mm to about 12 mm, about 6 mm to about 10 mm, or about 7 mm to about 8 mm (including all ranges and subranges therebetween). In contrast, a second dimension of the opening (e.g., width, or horizontal distance between first and second ends of the body) may be fixed, and may be about 0 mm to about 55 mm, such as about 5 mm to about 50 mm, about 10 mm to about 45 mm, about 15 mm to about 40 mm, about 20 mm to about 35 mm, or about 25 mm to about 30 mm (including all ranges and subranges therebetween). In some variations, a width of tissue clamped within the jaws may be about equal to or less than a width of the opening.

In some variations, the first jaw may be a superior jaw and the second jaw may be an inferior jaw according to their relative positions with respect to a head and feet of a patient. That is, the first jaw may be a superior or upper jaw because it may be configured to be positioned closer to the head, and farther from the feet, of the patient compared to the second jaw, which may thus be the inferior or lower jaw.

In some variations, the jaws of the tissue excision devices herein may be made of a same material as the bodies thereof, such as one or more polymers (e.g., polycarbonate, polypropylene). In some variations, a first jaw may include a first material, and a second jaw may include a second, different material. For example, the first jaw may include polypropylene, and the second jaw may include polycarbonate. In some instances, use of differing materials on the first and second jaws, such as on first and second tissue contact surfaces of the first and second jaws, may reduce a coefficient of friction between the two jaws, facilitating movement of the jaws against one another (e.g., movement of portion of the second jaw within portions of the first jaw, as explained below).

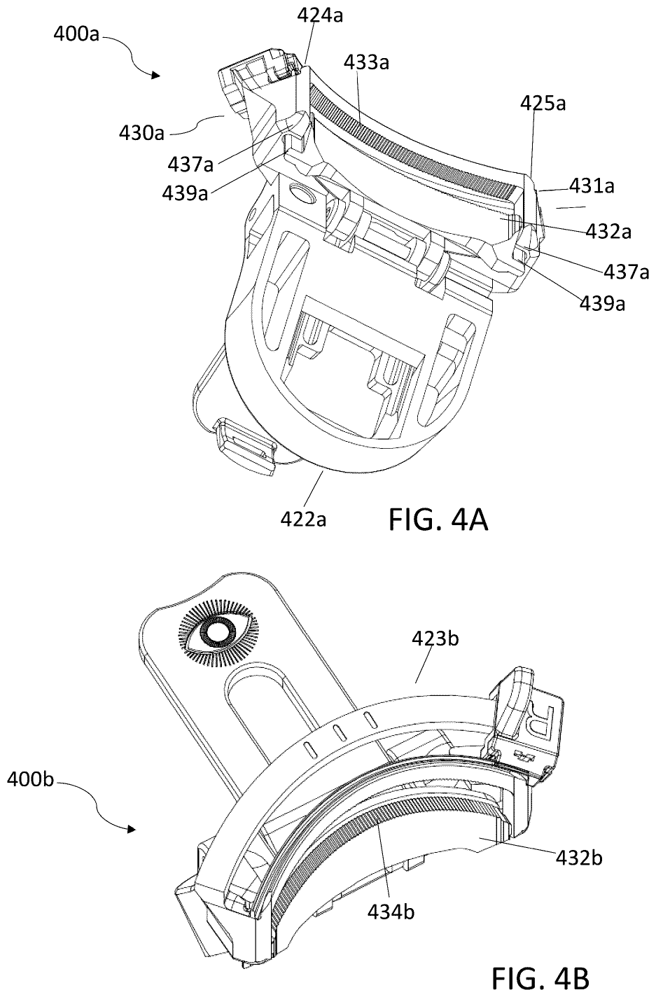

As noted above with respect to FIG. 2, in some cases, a second jaw of the jaws may be movably coupled to a first jaw of the jaws. For example, the first jaw may include coupling guides (not shown) configured to house one or more coupling features 239 of the second jaw such that the second jaw may be moved (e.g., vertically translated) relative to the first jaw. Such an example is illustrated in FIG. 4A, which depicts a back view of a tissue excision device 400a including jaws 430a with first jaw 431a and second jaw 432a. As shown, the second jaw 432a may include coupling features 439a, which may be projections or flanges extending laterally from the second jaw 432a. The coupling features 439a may be configured to be received within corresponding coupling guides 437a of the first jaw 431a, which may include channels or lumens at first and second lateral ends 424a, 425a of the device 400. The coupling guides 437a may be configured to partially surround and guide movement of the coupling features 439a therein. The first and second coupling features 439a may have any suitable shape that corresponds to a shape of the first and second coupling guides 437a or otherwise allows mating with the first and second coupling guides 437a, such as a cylindrical, substantially cylindrical, rectangular, or substantially rectangular shape.

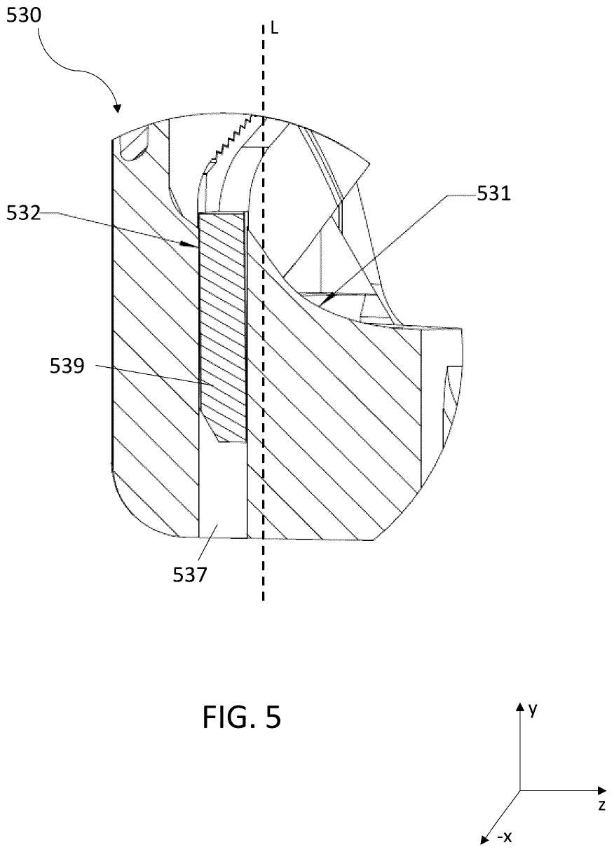

Another example of the coupling between first and second jaws of the tissue excision devices herein is depicted in FIG. 5, which is a cross-sectional view of jaws 530, including first jaw 531 and second jaw 532. In some variations, to reduce rotation of the second jaw 432a when it is moved relative to the first jaw 431a, the coupling features 539 of the second jaw 532 may be rotated with respect to a longitudinal axis L of the device, while the coupling guides 537 of the first jaw 531 may be aligned with the longitudinal axis L. For example, the coupling features 539 may be rotated about 0.05 degrees to about 5 degrees in the X- , Y- , and/or Z-directions, such as about 0.075 degrees to about 4 degrees, about 0.1 degrees to about 3 degrees, about 0.2 degrees to about 2.5 degrees, about 0.3 degrees to about 2 degrees, about 0.4 degrees to about 1.5 degrees, about 0.5 degrees to about 1 degree, or about 0.6 degrees to about 0.75 degrees (e.g., about 0.5, about 0.6, or about 0.7 degrees). Alternatively, in some variations, to reduce rotation of the second jaw 532 when it is moved relative to the first jaw 531, the coupling guides 537 may be rotated with respect to the longitudinal axis L of the device, while the coupling features 539 may be aligned with the longitudinal axis L. For example, the coupling guides 537 may be rotated about 0.05 degrees to about 5 degrees in the x- , y- , and/or z-directions, such as about 0.075 degrees to about 4 degrees, about 0.1 degrees to about 3 degrees, about 0.2 degrees to about 2.5 degrees, about 0.3 degrees to about 2 degrees, about 0.4 degrees to about 1.5 degrees, about 0.5 degrees to about 1 degree, or about 0.6 degrees to about 0.75 degrees (e.g., about 0.5, about 0.6, or about 0.7 degrees).

Moreover, to maximize a length of an opening defined by the first and second jaws 531, 532 in an open configuration, the second jaw 532 and/or the first jaw 531 may be configured to allow for freedom of movement of the coupling features 539 within the coupling guides 537. To do so, in some variations, the coupling features 539 may have a length along the longitudinal axis L of a tissue cutting device that may be about equal to or less than a length of the coupling guides 537 along the longitudinal axis L. For example, the length of the coupling features 539 may be about 25 mm to about 500 mm, such as about 50 mm to about 250 mm, about 75 mm to about 200 mm, or about 100 mm to about 150 mm (e.g., about 150 mm), and the length of the coupling guides 537 may be about 50 mm to about 750 mm, such as about 75 mm to about 500 mm, about 100 mm to about 250 mm, or about 125 mm to about 200 mm (e.g., greater than about 150 mm). Additionally, or alternatively, referring again to FIG. 4A, in some variations, the coupling guides 437a may not include distal stops or bases. That is, the coupling guides 437a may have open distal ends and thus may not restrict the coupling features 439a (or at least portions thereof) from being moved beyond the distal ends, or out of, the coupling guides 437a.

As noted above, the jaws may include one or more tissue contact surfaces for clamping tissue therebetween, such as a first tissue contact surface of the first jaw and a second tissue contact surface of the second jaw. In some variations, one or both of the first and second tissue contact surfaces may have a constant or varied depth of the tissue excision device (e.g., along a Z-axis of the device, according to the perspective of a user). In some variations, a tissue contact surface with a constant depth along the transverse axis of the tissue excision device may facilitate exertion of a uniform clamping force of the jaws on tissue held therein. An average or maximum depth of one or both of the first and second tissue contact surfaces may be about 0.25 mm to about 10 mm, such as about 0.5 mm to about 7.5 mm, about 0.75 mm to about 5 mm, about 1 mm to about 2.5 mm, about 1.25 mm to about 2.25 mm, or about 1.5 mm to about 2 mm (including all ranges and subranges therebetween). Moreover, a width (e.g., along a transverse or X-axis of the device, according to the perspective of a user) of one or both of the first and second tissue contact surfaces may be about 10 mm to about 500 mm, such as about 15 mm to about 275 mm, about 25 mm to about 250 mm, about 50 mm to about 225 mm, about 75 mm to about 200 mm, about 100 mm to about 175 mm, or about 125 mm to about 150 mm (including all ranges and subranges therebetween).

Exemplary tissue contact surfaces are also depicted in FIGS. 4A and 4B. Referring to FIG. 4A, the first jaw 431a of the jaws 430a may include a first tissue contact surface 433a, which may be serrated. The first tissue contact surface 433a may be oriented in a first direction, such as toward a first end 422a (e.g., a distal or bottom end, from a perspective of a user of the device 400a) of the device 400a. In some variations, the first tissue contact surface 433a may be a bottom surface of the first jaw 431a (as opposed to a top surface, such as top surface 136a,b of the first jaw 131a,b of FIGS. 1A and 1B). Referring to FIG. 4B, the second jaw 432b of the jaws 430b may include a second tissue contact surface 434b, which may be serrated. The second tissue contact surface 434b may be oriented in a second direction, such as toward a second end 423b (e.g., a proximal or top end, from a perspective of a user of the device 400b) of the device 400b. In some variations, the second tissue contact surface 434b may be a top surface of the second jaw 432b.

Furthermore, the jaws of the tissue excision devices herein may be curved. For example, the devices described herein, and particularly, the jaws, may have a compound curvature, which may facilitate alignment of the tissue excision devices with a curved tissue excision site and allow the tissue to be excised to be clamped with a force configured to cause hemostasis in the tissue to allow a wound caused by the excision to self-seal. For example, one or both of the first and second jaws of the tissue excision devices herein may include more than one curve, such as a first curve and a second curve on each of the first and second jaws. The first curve may be an anteroposterior curve that follows a contour of a tissue excision site (e.g., upper eyelid area) to facilitate a desired excision shape within the tissue excision site. Put another way, the anteroposterior curve may be symmetric around a central YZ-plane bisecting the device such that first and second lateral ends (relative to the X-axis) of the anteroposterior curve have the same Z-coordinates, and a midpoint of the anteroposterior curve has a larger (more positive) Z-coordinate. The second curve may be a superoinferior curve along one or more tissue contact surfaces of a tissue excision device (e.g., along first and second tissue contact surfaces of first and second jaws) and may be shaped to secure tissue so that blood flow is restricted to the tissue and an excision wound within the tissue may self-seal. In other words, the superoinferior curve may be symmetric about the central YZ-plane bisecting the device such that first and second lateral ends (relative to the X-axis) of the anteroposterior curve have the same Y-coordinates, and a midpoint of the anteroposterior curve has a larger (more positive) Y-coordinate.

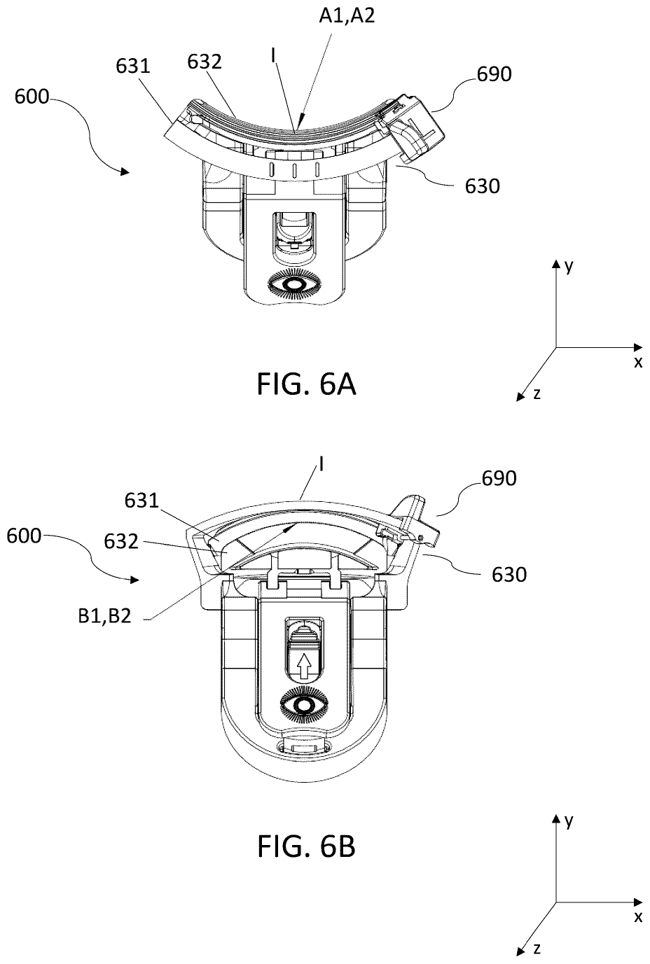

Such an example is depicted in FIGS. 6A and 6B, which show a top view and a front view of a tissue excision device 600, respectively. In FIG. 6A, a first jaw 631 and a second jaw 632 of tissue excision device 600 may each have first curves A1, A2, respectively. The first curves A1, A2 may be anteroposterior curves, which may be along a back side of the device and thus a back side of the first and second jaws 631, 632. In some variations, the first curves A1, A2 may be unique to each patient and/or tissue excision site, and may be established using patient anatomy imaging (e.g., using photographs, computerized tomography (CT) scans, 3D scanners, and/or data or information from other sources including, for example, other imaging modalities). The radii of curvature (ROC) of the first curves A1, A2 may be about 20 mm to about 100 mm, such as about 25 mm to about 80 mm, about 30 mm to about 60 mm, about 35 mm to about 50 mm, about 40 mm to about 45 mm, about 15 mm to about 60 mm, about 20 mm to about 50 mm, about 25 mm to about 40 mm, or about 30 mm to about 30 mm, including all ranges and subranges therebetween (e.g., 33 mm). In some variations, the ROC of the first curves A1, A2 may be about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm. In some variations, the first curve A1 of the first jaw 631 may be different than the first curve A2 of the second jaw 632. For example, an ROC of the first curve A1 may be less than, or greater than, an ROC of the first curve A2. In some variations, a difference between the ROCs of the first and second curves A1, A2 may be about 0.5 mm to about 10 mm, such as about 0.75 mm to about 5 mm, about 1 mm to about 2.5 mm, about 1.25 mm to about 2 mm, or about 1.5 mm to about 1.75 mm (including all ranges and subranges therebetween). Moreover, an arc length of one or both of the first and second jaws 631, 632 along the first curve A1, A2 may be about 20 mm to about 200 mm, such as about 25 mm to about 175 mm, about 30 mm to about 150 mm, about 40 mm to about 125 mm, about 50 mm to about 100 mm, or about 55 mm to about 75 mm (e.g., about 33 mm, about 55 mm).

In FIG. 6B, a second curve of the first jaw 631 and a second jaw 632 of tissue excision device 600 may each have second curves B1, B2, respectively. The second curves B1, B2 may be superoinferior curves, which may define the contours of first and second tissue contact surfaces (not shown) located on the first and second jaws 631, 632, respectively. That is, the second curves A1, A2 may be along a interior surfaces of, and thus “within”, the tissue excision device 600. In some variations, the second curves B1, B2 may be unique to each patient and/or tissue excision site, and may be established using patient anatomy imaging (e.g., using photographs, computerized tomography (CT) scans, 3D scanners, and/or data or information from other sources including, for example, other imaging modalities). The radii of curvature (ROC) of the second curves B1, B2 may be about 10 mm to about 90 mm, such as about 15 mm to about 80 mm, about 20 mm to about 70 mm, about 25 mm to about 60 mm, about 30 mm to about 50 mm, or about 35 mm to about 45 mm (including all ranges and subranges therebetween). In some variations, the ROC of the second curves B1, B2 may be about 30 mm, about 31 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm. In some variations, an ROC of second curve B1 of the first tissue contact surface may be different than an ROC of the second curve B2 of the second tissue contact surface. For example, the ROC of the second curve B1 may be less than, or greater than, the ROC of the second curve B2. In some variations, a first ROC of the second curve B1 may be about 20 mm to about 60 mm, such as about 25 mm to about 50 mm, or about 30 mm to about 40 mm (including all ranges and subranges therebetween). In some variations the first ROC of the second curve B1 may be less than about 30 mm, about 30 mm, about 31 mm, about 32 mm, about 33 mm, about 34 mm, about 35 mm, about 36 mm, about 37 mm, about 38 mm, about 39 mm, about 40 mm, or greater than about 40 mm (e.g., about 34 mm to about 35 mm). Moreover, a second first ROC of the second curve B2 may be about 10 mm to about 50 mm, such as about 15 mm to about 45 mm, about 20 mm to about 40 mm, or about 25 mm to about 35 mm (including all ranges and subranges therebetween). In some variations the second ROC of the second curve B2 may be less than about 20 mm, about 20 mm, about 21 mm, about 22 mm, about 23 mm, about 24 mm, about 25 mm, about 26 mm, about 27 mm, about 28 mm, about 29 mm, about 30 mm, about 30.5 mm, about 31 mm, about 31.5 mm, about 32 mm, about 32.5 mm, about 33 mm, about 33.5 mm, about 34 mm, about 34.5 mm, about 35 mm, or greater than about 35 mm. A difference between the ROC of the second curve B1 and the ROC of the second curve B2 may be about 0 mm to about 10 mm, such as about 0.5 mm to about 9 mm, about 1 mm to about 8 mm, about 1.25 mm to about 7 mm, about 1.5 mm to about 6 mm, about 1.75 mm to about 5.5 mm, about 2 mm to about 5 mm, about 2.25 mm to about 4.75 mm, about 2.5 mm to about 4.5 mm, about 2.75 mm to about 4.25 mm, about 3 mm to about 4 mm, or about 3.25 mm to about 3.75 mm (including all ranges and subranges therebetween). Similarly, in some variations, the ROC of the second curve B1 may be about 0.01 to about 5 times greater than the ROC of the second curve B2, such as about 0.025 to about 4 times greater, about 0.05 to about 3 times greater, about 0.075 to about 2.5 times greater, about 0.1 to about 2 times greater, about 0.25 to about 1.9 times greater, about 0.5 to about 1.8 times greater, about 0.75 to about 1.75 times greater, about 1 to about 1.5 times greater, about 1.01 to about 1.25 times greater, about 1.02 to about 1.1 times greater, about 1.03 to about 1.09 times greater, about 1.04 to about 1.08 times greater, about 1.045 to about 1.075 times greater, about 1.05 to about 1.07 times greater, about 1.055 to about 1.065 times greater (e.g., about 1.04 to about 1.045, or about 1.04 to about 1.05 times greater, including all ranges and subranges therebetween).

Moreover, an arc length of one or both of the first and second jaws 631, 632 along the second curve B1, B2 may be about 20 mm to about 200 mm, such as about 25 mm to about 175 mm, about 30 mm to about 150 mm, about 40 mm to about 125 mm, about 50 mm to about 100 mm, or about 55 mm to about 75 mm (e.g., about 55 mm).

Furthermore, the jaws herein may comprise one or more features configured to engage or interface with another component of the tissue excision devices, such as with the cutting assembly or with a cutting assembly lock. For example, one or both of a first jaw and a second jaw may comprise a cutting guide configured to couple to and guide a cutting assembly through tissue secured by a tissue excision device to excise the tissue. In some variations, the cutting guide may comprise one or more elongate slots, tracks, or edges along a first or second jaw of a tissue excision device. The cutting guide may be curved, and the curve may match the curvature of the either the first jaw or the second jaw carrying the cutting guide.

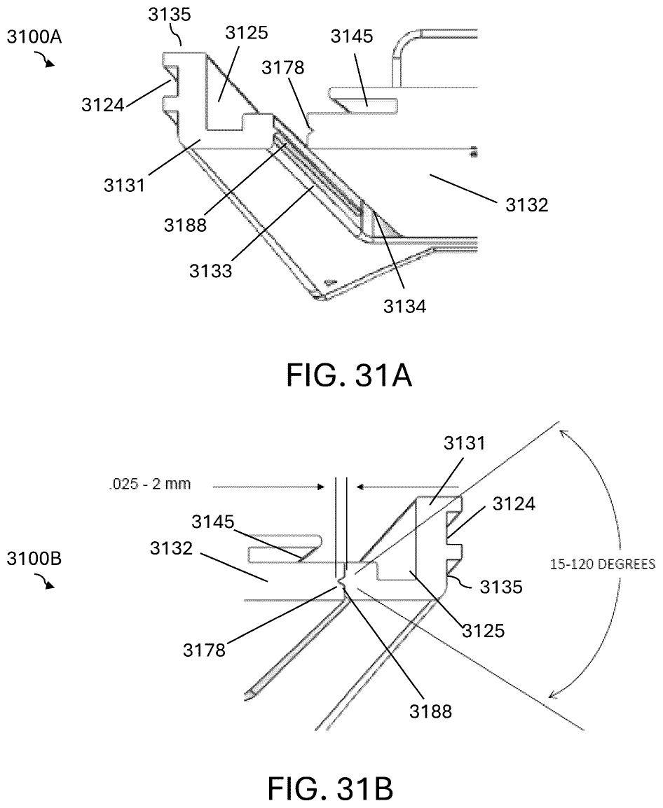

In some variations, the jaws may comprise complementary engagement features to enhance tissue clamping, promote hemostasis, and provide precise alignment during actuation. FIGS. 31A and 31B depict tissue excision device 3100A/B in open and closed configurations, respectively, showing exemplary jaw engagement features. In this variation, the first jaw 3131 and second jaw 3132 define respective tissue contact surfaces 3133 and 3134. The first jaw 3131 may comprise a first engagement feature 3188, such as a male engagement feature (e.g., ridge, tongue, other raised structure), and the second jaw 3132 may comprise a second engagement feature 3178, such as female engagement feature (e.g., groove, recess, other depressed feature), configured to receive the tongue therein. These complementary engagement features may create a mechanical interlock when the jaws are closed, thereby providing enhanced stability and consistent tissue compression.

The first engagement feature 3188 may comprise a male engagement feature (e.g., a raised structure like a tongue) extending along at least a portion of the length of the first tissue contact surface 3133. The height of the male engagement feature may range from about 0.025 mm to about 3 mm, such as about 0.05 mm to about 2.5 mm, about 0.1 mm to about 2 mm, about 0.15 mm to about 1.5 mm, about 0.2 mm to about 1 mm, or about 0.25 mm to about 0.75 mm (including all ranges and subranges therebetween). The profile of the male engagement feature may be triangular, trapezoidal, semicircular, or rectangular, with any suitable radius of curvature at transitional edges to reduce tissue trauma. In some variations, the male engagement feature may comprise an angular (e.g., triangular) cross-sectional shape. An angle thereof may range from about 15 degrees to about 120 degrees, such as about 30 to 100 degrees, or about 45 to 90 degrees, and may be substantially equal to an angle of a corresponding female engagement feature. In some variations, the male engagement feature may comprise a varying height profile along its length to accommodate the varying geometries of the jaws or to provide different compression forces at different points along the tissue contact surfaces.

The second engagement feature 3178 may comprise a female engagement feature (e.g., a depressed structure like a groove) configured to receive and mate with the first engagement feature 3188. The depth of the female engagement feature may correspond to the height of the male engagement feature, and may range from about 0.025 mm to about 3.5 mm, such as about 0.075 mm to about 3 mm, about 0.1 mm to about 2.5 mm, about 0.2 mm to about 2 mm, about 0.3 mm to about 1.5 mm, or about 0.4 mm to about 1 mm (including all ranges and subranges therebetween). The profile of the female engagement feature profile may be complementary to that of the male engagement feature to provide optimal mating, or may have a different profile to create specific tissue compression patterns. For example, a trapezoidal tongue may mate with a rectangular groove to create focused compression zones. In some variations, the female engagement feature may comprise an angular (e.g., triangular) cross-sectional shape. An angle thereof may range from about 15 degrees to about 120 degrees, such as about 30 to 100 degrees, or about 45 to 90 degrees, and may be substantially equal to an angle of the male engagement feature. In some variations, the male engagement feature may comprise a varying depth profile along its length to accommodate the varying geometries of the jaws or to provide different compression forces at different points along the tissue contact surfaces.

The first and second engagement features 3188, 3178 shown in FIGS. 31A and 31B may extend along any suitable portion of the length of the respective tissue contact surfaces. In some variations, the engagement features may extend along the entire length of the tissue contact surfaces, while in other variations, they may extend along about 25% to about 95% of the length, such as about 30% to about 90%, about 40% to about 85%, about 50% to about 80%, or about 60% to about 75% (including all ranges and subranges therebetween). When the jaws transition from the open configuration shown in FIG. 31A to the closed configuration shown in FIG. 31B, the first and second engagement features 3188, 3178 may align and mate, creating a mechanical interlock that enhances jaw stability and tissue compression.

In some variations, the positions of the engagement features may be reversed, with the first jaw 3131 comprising the female engagement feature and the second jaw 3132 comprising the male engagement feature. Additionally, in some variations, multiple ridge-and-groove pairs may be provided along the tissue contact surfaces to create alternating compression zones or to enhance tissue grip. The transverse positioning of the engagement features may also vary, with options including central placement along the tissue contact surfaces, offset placement, or multiple parallel features.

The tissue contact surfaces 3133, 3134 in FIGS. 31A and 31B may be smooth in some variations, relying primarily on the ridge-and-groove engagement to secure tissue. In other variations, the tissue contact surfaces may comprise additional texturing, such as serrations, knurling, or micro-texturing, to further enhance tissue grip. These textured regions may be provided across the entire tissue contact surface or may be selectively applied to regions adjacent to or distant from the engagement features.

In addition to the engagement features, FIGS. 31A and 31B illustrate a cutting element guide/slot 3145 configured to receive a cutting element during tissue excision. The cutting element guide 3145 may be formed in the second jaw 3132 and may extend along a length thereof. The slot 3145 may have a depth of about 0.1 mm to about 5 mm, such as about 0.25 mm to about 4 mm, about 0.5 mm to about 3 mm, about 0.75 mm to about 2.5 mm, or about 1 mm to about 2 mm (including all ranges and subranges therebetween). The slot width may be precisely sized to accommodate the thickness of the cutting element with minimal lateral play, thereby ensuring accurate cutting. In some variations, the slot may comprise tapered or radiused edges to facilitate smooth entry and movement of the cutting element. The slot 3145 may follow the curvature of the second jaw 3132, maintaining a consistent depth along its length to ensure uniform cutting action.

FIGS. 31A and 31B further illustrate a cutting assembly guide with first and second guide rails/tracks 3124, 3125 positioned on the first jaw 3131. These guide tracks may be precisely positioned to align with and guide a cutting assembly along a predetermined path during tissue excision. The first and second guide tracks 3124, 3125 may be oriented substantially parallel to one another and may be spatially configured to receive corresponding portions of a cutting assembly. The guide tracks may be integrally formed with the first jaw or may be separately attached components. In some variations, the guide tracks may comprise smooth surfaces to facilitate low-friction movement of the cutting assembly, while in other variations, they may comprise textured or grooved surfaces to enhance cutting assembly retention.

The dimensions and orientation of the guide tracks 3124, 3125 may be optimized to accommodate specific cutting assembly geometries. The distance between the tracks may range from about 2 mm to about 25 mm, such as about 3 mm to about 20 mm, about 4 mm to about 15 mm, about 5 mm to about 12 mm, or about 6 mm to about 10 mm (including all ranges and subranges therebetween). The height of each track may range from about 0.25 mm to about 5 mm, such as about 0.5 mm to about 4 mm, about 0.75 mm to about 3 mm, about 1 mm to about 2.5 mm, or about 1.25 mm to about 2 mm (including all ranges and subranges therebetween).

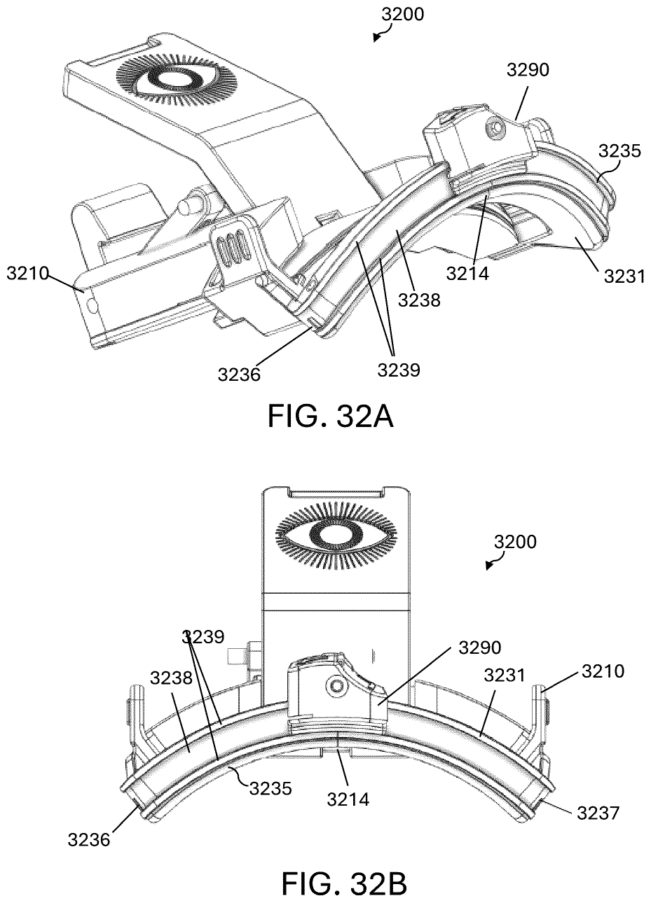

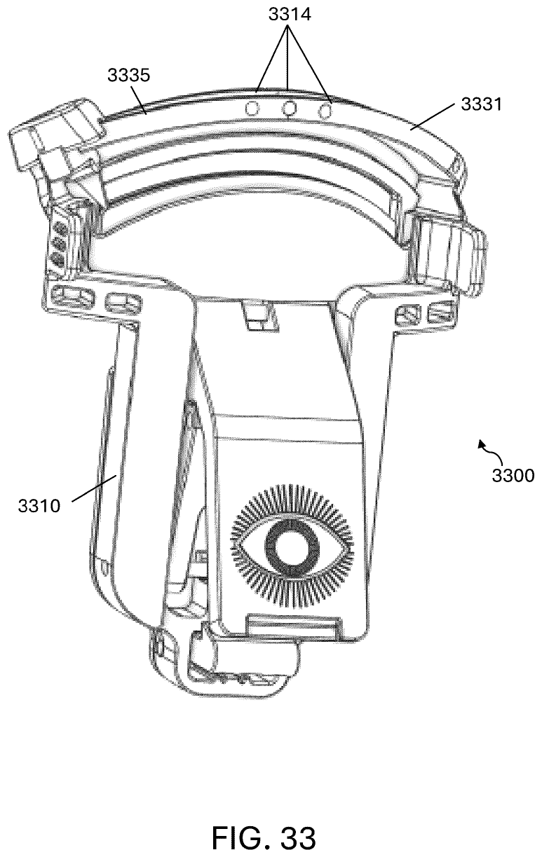

Furthermore, the tissue excision devices herein may comprise one or more alignment features and detent mechanisms to enhance device positioning and operation. FIGS. 32A and 32B depict perspective and top views, respectively, of a tissue excision device 3200 comprising a body 3210 and a first jaw 3231 defining a curved cutting guide 3235, with a cutting assembly 3290 disposed thereon. The device further may further comprise a center marking 3214 on the cutting guide 3235 to facilitate alignment of the cutting assembly 3290 in a centered position. This marking may serve as a visual alignment indicator for ensuring symmetrical placement of the device on the patient and may be configured as a line, dot, arrow, or other suitable indicator. Such markings are described in more detail with reference to FIG. 33 below.

The cutting guide 3235 may further comprise one or more first engagement features 3236, such as recesses, notches, or grooves formed in an upper, outer, or lateral surface of the guide. These features may be configured to receive and releasably engage with corresponding second engagement features on the cutting assembly 3290. The engagement features 3236 may have dimensions ranging from about 0.1 mm to about 10 mm in depth and about 0.25 mm to about 5 mm in width, such as about 0.25 mm to about 7.5 mm in depth and about 0.5 mm to about 4 mm in width, or about 0.5 mm to about 5 mm in depth and about 0.75 mm to about 3 mm in width. These engagement features may be spaced regularly or irregularly along the cutting guide 3235, and may be distributed with varying density depending on the intended use of the device.

Additionally, the cutting guide 3235 may comprise at least one channel 3238 defined by sidewalls 3239 that extend from a top/distalmost surface of the first jaw 3231. This channel structure may be configured to provide enhanced guidance and stability for the cutting assembly during actuation. The channel 3238 may have a depth of about 0.5 mm to about 8 mm, such as about 1 mm to about 7 mm, about 1.5 mm to about 6 mm, about 2 mm to about 5 mm, or about 2.5 mm to about 4 mm (including all ranges and subranges therebetween). The width of the channel may be precisely sized to accommodate the corresponding portion of the cutting assembly with minimal lateral play, thereby ensuring accurate tracking during the tissue excision procedure.

FIG. 33 depicts a perspective view of a tissue excision device 3300 comprising a body 3310 and a first jaw 3331 defining a cutting guide 3335. The device further comprises alignment markings 3314 provided on the cutting guide and/or adjacent surfaces of the first jaw. These markings may serve as visual reference indicators for device positioning relative to anatomical landmarks or surgical planning marks. The alignment markings 3314 may comprise a variety of shapes, patterns, or symbols, such as lines, dots, crosses, or alphanumeric characters, and may be symmetrically distributed along the jaw to facilitate midline alignment. Any suitable number of alignment markings 3314 may be used, such as 1, 2, 3, 4, 5, 6, 7, 8, 9, 10, or more than 10 markings. In some variations, the device 3300 may comprise an odd number of alignment markings 3314 (e.g., 1, 3 or 5) to aid in symmetrical alignment of the device 3300 against the tissue excision site.

The alignment markings 3314 may be applied using various manufacturing techniques, such as laser etching, pad printing, molding, or other suitable processes that ensure visibility and durability throughout the procedure. In some variations, the markings may be color-coded to correspond with different anatomical features or may comprise gradations to indicate measuring increments. The markings may be particularly valuable in procedures requiring precise alignment with facial features, such as brow position, eyelid creases, or commissures, thereby enhancing the consistency and predictability of outcomes.

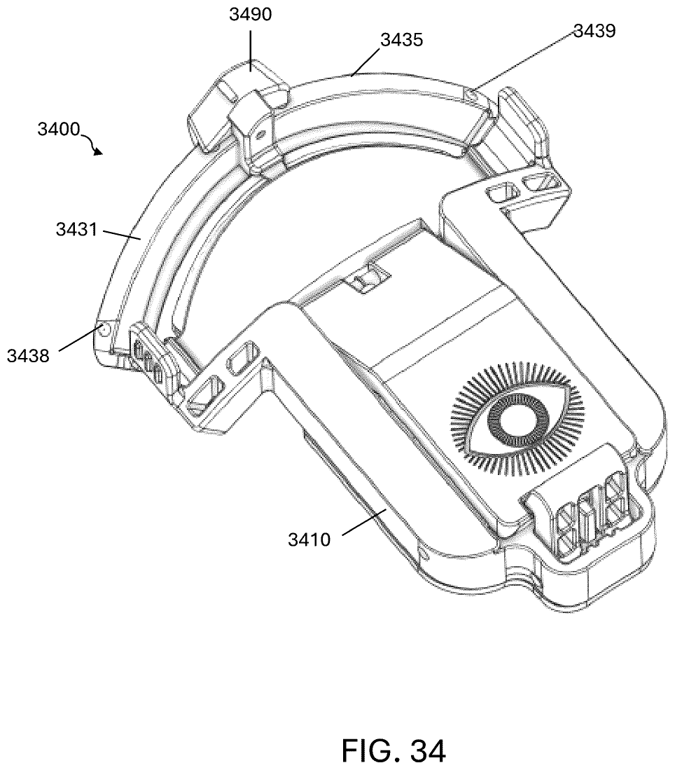

FIG. 34 illustrates a perspective view of an exemplary tissue excision device 3400 comprising a body 3410, a first jaw 3431, and a cutting guide 3435 disposed along the first jaw. The device further comprises first and second detents 3438, 3439 formed along the cutting guide or adjacent regions of the first jaw. These detents may comprise mechanical features configured to positively engage the cutting assembly at specific points along its travel path, such as at the beginning and the end, thereby creating defined starting and stopping positions.

The first detent 3438 may correspond to an initial position of the cutting assembly prior to the excision procedure, while the second detent 3439 may correspond to a final position following completion of the excision. The detents may comprise various geometries, such as recesses, protrusions, or combinations thereof, configured to provide both tactile and audible feedback when engaged. The dimensions of the detents may range from about 0.1 mm to about 2.5 mm in height or depth and about 0.25 mm to about 3.5 mm in width, such as about 0.2 mm to about 2 mm in height or depth and about 0.5 mm to about 3 mm in width, or about 0.3 mm to about 1.5 mm in height or depth and about 0.75 mm to about 2.5 mm in width (including all ranges and subranges therebetween).

The spacing between the first and second detents 3438, 3439 may correspond to the full excision path length, which may range from about 20 mm to about 100 mm, such as about 25 mm to about 90 mm, about 30 mm to about 80 mm, about 35 mm to about 70 mm, about 40 mm to about 65 mm, about 45 mm to about 60 mm, or about 50 mm to about 55 mm (including all ranges and subranges therebetween). In some variations, additional intermediate detents may be provided to create staged cutting sequences or to provide positional feedback during the procedure.

In some variations, the jaws herein, such as one or both of first and second jaws of a tissue excision device, may comprise a cutting assembly lock attached thereto. The cutting assembly lock may be configured to prevent movement of a cutting element of a cutting assembly of the tissue excision device after the cutting assembly is moved from a first position to a second position along the jaws (e.g., along the first jaw). In some variations, the first position may be an initial position of the cutting assembly (e.g., proximal to or at a first end of the tissue excision device) prior to tissue excision and the second position may be a final position of the cutting assembly (e.g., proximal to or at a second end of the tissue excision device) after tissue excision. In some variations, the cutting assembly lock may be fixed to (e.g., integrally formed with) the jaws (e.g., one or both of the first and second jaws). The cutting assembly lock may releasably engage with one or more components of the cutting assembly, such as, for example, a housing of the cutting assembly. For example, the cutting assembly lock may comprise a pin or other protrusion that is received within a corresponding opening or indentation within the housing, and/or may comprise a cutting element retention feature configured to receive or otherwise engage with the cutting element.

Moreover, the jaws herein may comprise one or more features, aside from the tissue contact surfaces, configured to engage or interface with tissue to be excised. For example, one or both of the first jaw and the second jaw of the tissue excision device may comprise a slot or channel (also referred to herein as a “cutting element guide”) configured to receive a cutting element of the cutting assembly to further guide and stabilize the cutting element during tissue excision. In some variations, the slot may be an elongate slot within a second jaw of a tissue excision device. The slot may or may not be curved. When the jaws are in the closed/clamped position, at least a portion of the cutting element may be within the cutting element guide or slot.

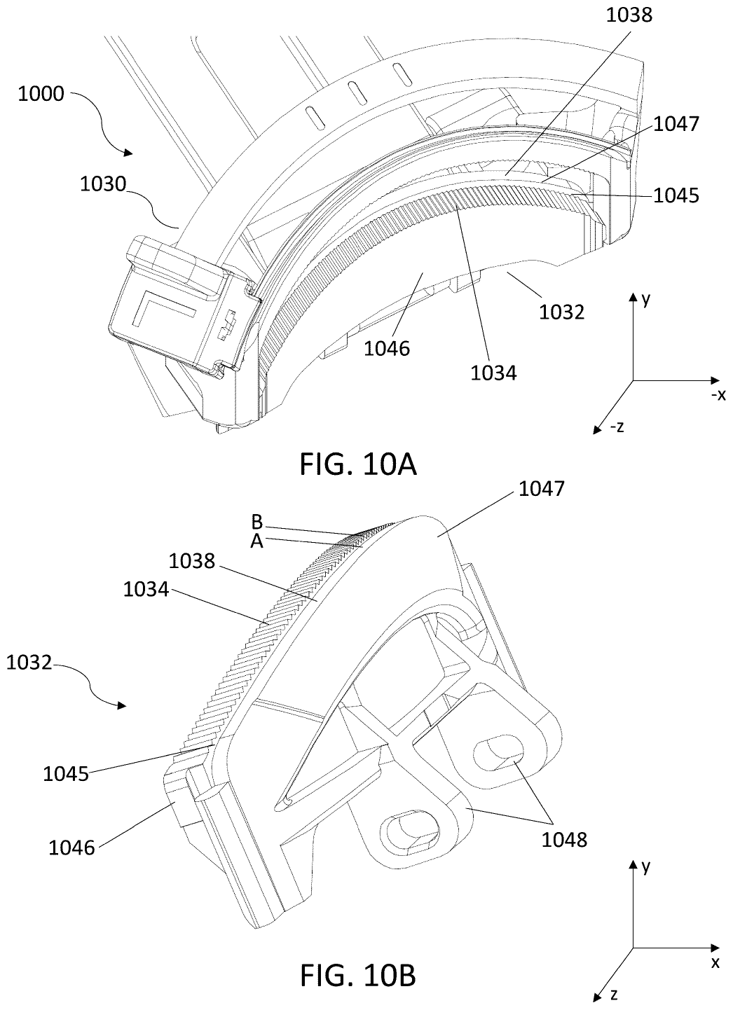

For example, as shown in FIG. 10A, a second jaw 1032 of jaws 1030 of a tissue excision device 1000 may include a slot 1045. The slot 1045 may be formed between a first wall 1046 (e.g., an inner wall) of the second jaw 1032 and a second wall 1047 (e.g., an outer wall) of the second jaw 1032. The first wall 1046 may be an inner wall and the second wall 1047 may be an outer wall in that the first wall 1046 may be positioned closer to a patient or tissue excision site than the second wall 1047 when the tissue excision device 1000 is in use during a tissue excision procedure. Put another way, the first and second walls 1046, 1047 may be defined relative to a plane, such as a plane within the XYZ coordinate system shown. Here, the Y-axis may be defined by a central longitudinal axis of the tissue excision device 1000, and the first and second walls 1046, 1047 may be aligned along an XY-plane that longitudinally bisects the tissue excision device 1000. Because FIG. 10A is a back view of the tissue excision device 1000, the first wall 1046 may be positioned further along the Z-axis (in the-Z direction) than the second wall 1047. It should be understood that the relative positions of the first and second walls 1046, 1047 may alternatively depend on a position of the tissue excision device 1000 in space, and thus may additionally be described relative to the YZ-plane and/or the ZX-plane. An offset (e.g., along a Z-axis, according to a perspective of a user during tissue excision) between the first and second walls 1046, 1047 may define the slot 1045. The slot 1045 may be configured to receive a portion or at least a portion of a cutting element (not shown) of a cutting assembly 1090. In some variations, a first dimension of the slot 1045, such as a depth along the Z-axis (from a perspective of a user), may be about equal to or greater than a maximum thickness of a cutting element (not shown) of the cutting assembly 1090. For example, the depth of the slot 1045 may be about 0.05 mm to about 5 mm, such as about 0.1 mm to about 2.5 mm, about 1 mm to about 2 mm, or about 1.25 mm to about 1.75 mm (e.g., about 0.75 mm, about 0.8 mm, about 0.85 mm, about 0.9 mm, about 0.95 mm, about 1 mm, about 1.05 mm, about 1.1 mm, about 1.15 mm, about 1.2 mm, about 1.25 mm, about 1.3 mm, about 1.25 mm, about 1.4 mm, about 1.45 mm, or about 1.5 mm). In some variations, a second dimension of the slot 1045, such as a length along the longitudinal or Y-axis (from a perspective of a user), may be about equal to or less than a length of a cutting edge of the cutting element. For example, the length of the slot 1045 may be about 1 mm to about 50 mm, such as about 5 mm to about 25 mm, or about 10 mm to about 15 mm (including all ranges and subranges therebetween).

Referring again to the first and second walls, 1046, 1047, and now to FIG. 10B depicting second jaw 1032, the first wall 1046 may include a tissue contact surface (e.g., a second tissue contact surface) 1034, and the second wall 1047 may include a tissue support surface 1038. The tissue support surface 1038 may be configured to facilitate tissue excision by supporting or propping up excess tissue to be excised when the jaws (e.g., jaws 1030 of FIG. 10A) are clamped around the tissue. In some variations, the tissue support surface 1038 may be raised relative to the tissue contact surface 1034. For example, referring to the XYZ coordinate system shown in FIG. 10B, the tissue support surface 1038 may have a position (e.g., a maximum position) A along the Y-axis (e.g., vertical axis) that is greater than a position B (e.g., a maximum position) of the tissue contact surface 1034 along the Y-axis. Alternatively, in some variations, the tissue support surface 1038 may be level with the tissue contact surface 1034.

Moreover, the first and second walls 1046,1047 of the second jaw 1032 may each have a compound curvature, such as an anteroposterior and a superoinferior curvature. In some variations, both the first and second walls 1046, 1047 may have the same ROC for both of an anteroposterior curve and a superoinferior curve of each of the first and second walls 1046, 1047. When a tissue excision device including the second jaw 1032 (i.e., tissue excision device 1000 of FIG. 10A) is in-use, the anteroposterior curve may curve around a bisecting YZ-plane and have first and second lateral ends (relative to the X-axis) that are toward the-Z direction, and a midpoint that is toward the Z direction. Meanwhile, the superoinferior curve may curve around the central bisecting YZ-plane and have first and second lateral ends that are toward the-Y direction, and a midpoint that is toward (e.g., raised along) the Y direction.

As is also shown in FIG. 10B, the second jaw 1032 may include one or more pivot openings 1048 configured to couple to an actuator of the tissue excision device 1000. For example, the second jaw 1032 may include 1, 2, 3, 4, 5, 6, 7, 8, 9, 10, or greater than 10 pivot openings.

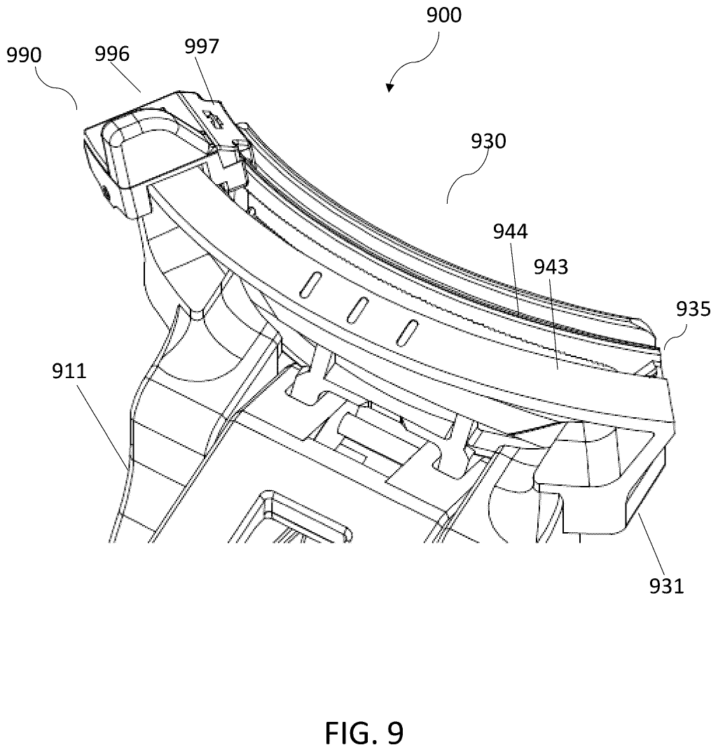

For example, as shown in FIG. 9, a cutting guide 935 may be fixed to (e.g., integrally formed with) a first jaw 931 of a tissue excision device 900. The cutting guide 935 may include a first track 943 configured to support a first portion 996 of a cutting assembly 990 and a second track 944 configured to support a second portion 997 of the cutting assembly 990, which may be movable (e.g., slidable) along the cutting guide 935. Each of the first and second tracks 943, 944 may be configured to be received within a recess of each of the first and second portions 996,997, respectively, of the cutting assembly 990. In some variations, a thickness (e.g., along a Z-axis of the device, from the perspective of a user) of the first track 943 may be about equal to or less than a thickness of the recess within the first portion 996. The thickness of the first track may be about 1 mm to about 20 mm, such as about 2.5 mm to about 15 mm, or about 5 mm to about 10 mm (including all ranges and subranges therebetween). Similarly, in some variations, the thickness of the second track 944 may be about equal to or less than a depth of the recess within the second portion 997. The depth of the second track may be about 0.1 mm to about 5 mm, such as about 0.2 mm to about 2.5 mm, about 0.3 mm to about 2.25 mm, about 0.4 mm to about 2 mm, about 0.5 mm to about 1.75 mm, about 0.6 mm to about 1.25 mm, or about 0.7 mm to about 1 mm. In some variations, a thickness of the first track 943 may be greater than a depth of the second track 944. Moreover, an ROC of one or both of the first and second tracks 943, 944 may be about equal to an ROC of the first jaw 931, such as an ROC of a first curve (e.g., an anteroposterior curve) of the first jaw 931. In some variations, the cutting guide 935 may be configured to position the cutting assembly 990 in close proximity with a front side 911 of the tissue excision device 900 such that a cutting element (not shown) of the cutting assembly may excise a majority of excess tissue clamped within the jaws 930. In some variations, a gap between the cutting element and the front side 911 may be about 0 mm to about 2 mm, such as about 0.1 mm to about 1.75 mm, about 0.2 mm to about 1.5 mm, about 0.3 mm to about 1.25 mm, about 0.4 mm to about 1 mm, or about 0.5 mm to about 0.75 mm (including all ranges and subranges therebetween). In some variations, the gap may be about 0.3 mm to about 0.6 mm, such as about 0.3 mm, about 0.4 mm, about 0.5 mm, or about 0.6 mm.

The jaws herein may be actuated between open and closed/clamped positions via a linkage assembly, which is described in detail below.

### Linkage Assembly

The tissue excision devices herein may comprise linkage assemblies that enable actuation of the jaws with a clamping force that may stop blood flow to the target tissue and/or yield a tissue seam. Various linkage assemblies may be utilized, such as a linkage assembly comprising any combination of an actuator to move the jaws from a first configuration to a second configuration (such as from an open configuration to a closed configuration or vice versa), a lock configured to maintain a second configuration of the jaws (such as the closed configuration), and a biasing member. The linkage assembly may be directly coupled to a movable jaw (e.g., the second/lower jaw described in detail above) of the tissue excision device.

The actuator may include one or more pivot openings coupling the actuator to the jaws herein (e.g., a second jaw) to control a relative position of the jaws, such as a position of a second jaw relative to a first jaw of a tissue excision device. For example, actuating a pivot rotation via one or more pivot joints of the actuator may allow for linear adjustment (e.g., along a longitudinal or vertical axis of the tissue excision device) between the first and second jaws, and thus between first and second tissue contact surfaces thereof. The actuator may be actuated (e.g., translated, rotated (e.g. pivoted) relative to a body of the tissue excision device, as well as relative to a lock coupled to the body.

In some variations, the actuator may be part of a broader linkage assembly. The linkage assembly may include the actuator, a linkage arm, one or more biasing members, and optionally a baseplate or jaw stop feature. The actuator may comprise a lever comprising a first end configured to pivotally couple to the second jaw and a second end configured to be acted upon by a user to cause motion. The lever may comprise a curved, bent (in one or more places), or straight profile. A central region of the actuator may be rotatably coupled to the body via a pivot pin, and may also provide coupling to the linkage arm. The actuator may include a first engagement feature, such as a recess, and an integrated stop member positioned near its first end. The stop member may comprise an angled protrusion configured to contact an underside of the second jaw to limit movement away from the first jaw.

The linkage arm may be pivotally coupled at a first end to the actuator (e.g., adjacent to a bend of the bent lever) and at a second end to the body. In some variations, the linkage arm may comprise an integrated stop member configured to engage an underside of the actuator. This stop member may act to limit rotation of the actuator and maintain a contact surface of the actuator against the second jaw. For example, the stop member of the linkage arm may prevent the actuator from rotating beyond a certain threshold angle, thereby ensuring a consistent open jaw position. In some variations, the stop member may be formed as an angled protrusion from an upper portion of the linkage arm.

Biasing of the actuator may be provided by a (first) biasing member, such as a spring. In some variations, the biasing member may comprise a tension spring coupled between the actuator and the body. For example, the spring may have a first end attached to the actuator (e.g., near the coupling to the linkage arm) and a second end attached to a fixed point within the body. The biasing member may bias the actuator toward an open position in which the second jaw is spaced apart from the first jaw. In other variations, a second biasing member (e.g., torsion spring) may be used to bias the actuator or lock into or out of engagement with each other.

Further, the lock may define freedom of movement of the actuator. The lock and actuator may be configured to releasably engage each other via corresponding engagement features. The lock may be configured to exert a force (e.g., a compression force) to maintain an engagement between the engagement features of the lock and the actuator. Additionally, the lock may be configured to receive an opposite force to release the engagement. A biasing member (e.g., a spring such as a torsion or tension spring) may be coupled with the lock to enable its actuation.

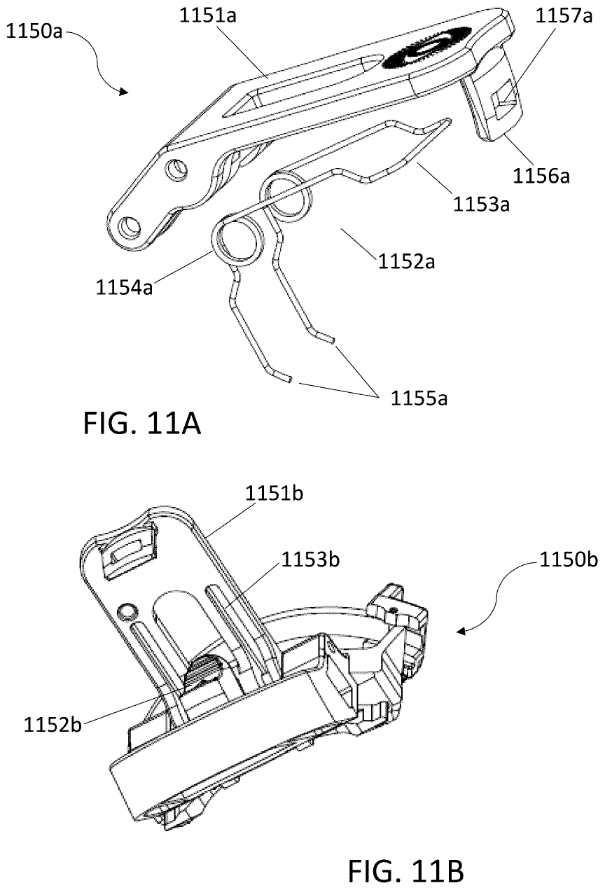

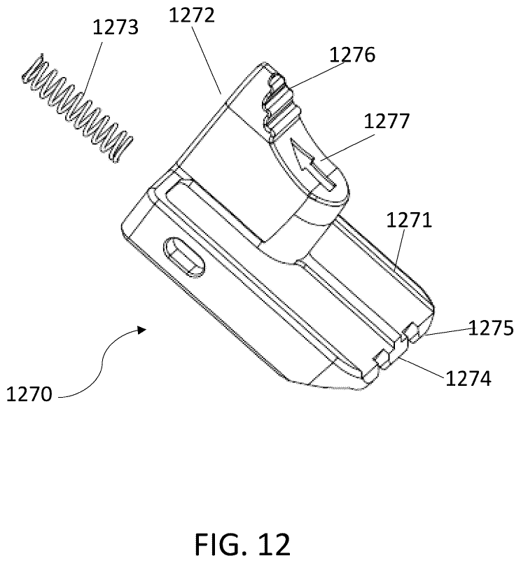

Components of each of an exemplary actuator 1150a and lock 1270 are shown in FIGS. 11 and 12, respectively. First, with respect to FIG. 11A, the actuator 1150a may include an actuator body 1151a, which may, in some variations, be a lever. The actuator body 1151a may be coupled to a spring 1152a. For example, a base 1153a of the spring 1152a may be carried within the actuator body 1151a. An example of this attachment is shown in FIG. 11B, where the base 1153b of the spring 1152b is housed within the body 1151b of the actuator 1150b. Referring again to FIG. 11A, one or more pivot openings 1154a of the spring 1152a may be coupled to (e.g., wound about) one or more pivot pins (e.g., pivot pin 216 of FIG. 2) to form a pivot joint that couples the actuator 1150a to a tissue excision device body, and one or more legs 1155a of the spring 1152a may be coupled to a back side of a lock of the tissue excision device (e.g., lock 1270 of FIG. 12). Further, the actuator body 1151a may include one or more pivot openings configured to couple to (e.g., via pins) one or more corresponding pivot openings of jaws of the tissue excision device to operably couple the actuator 1150a to the jaws. In some variations, the spring 1152a may be a torsion spring configured to bias the body 1151a in an open or unlocked configuration relative to a body or lock of the tissue excision device. The open configuration of the body 1151a (e.g., of the actuator 1150) may be defined by a first (e.g., transverse, perpendicular) position of the body 1151a relative to a longitudinal or vertical axis of the body of the tissue excision device. Oppositely, a closed or locked configuration of the body 1151a (e.g., of the actuator 1150) may be defined by a (e.g., longitudinal, parallel) position of the body 1151a relative to the longitudinal or vertical axis of the body of the tissue excision device. In some variations, the actuator body 1151a may be rotated about 20 degrees to about 160 degrees from the open configuration to the closed configuration relative to the body of the tissue excision device, such as about 30 degrees, about 45 degrees, about 60 degrees, or about 90 degrees. Moreover, the actuator 1150a may include an engagement feature 1156a extending from the actuator body 1150a (e.g., extending perpendicularly therefrom). The engagement feature 1156a may be configured to releasably engage a portion of the lock via a distal aperture 1157a. That is, the engagement feature 1156a may be configured to receive a portion of the lock (e.g., a corresponding engagement feature) through the distal aperture 1157a to secure the actuator 1150a in a closed configuration.

Second, referring to FIG. 12, the lock 1270 may include a lock body 1271, a lock actuator coupled (e.g., fixed) to the lock body 1271, a spring 1273, and an engagement feature 1274. The lock body 1271 may include a backside recess (not shown) configured to carry the spring 1273, which may be a compression spring. The lock actuator 1272 may extend from the body 1271 (e.g., transversely, perpendicularly therefrom), and may be configured to move (e.g., translate) the body 1271 relative to a body of a tissue excision device. In some variations, the lock actuator 1272 may be integrally formed with the body 1271. The engagement feature 1274 may be a projection or extension at a first end 1275 of the body 1271. The engagement feature 1274 may extend from the body 1271 (e.g., parallelly therefrom), and may be coupled to the lock actuator 1272 via the spring 1273. In some variations, the engagement features 1274 may be integrally formed with the body 1271. Additionally, the engagement feature 1274 may have a first configuration, such as an extended configuration (e.g., relative to a body of a tissue excision device), and a second configuration, such as a receded configuration (e.g., relative to a body of a tissue excision device), that is controllable via actuation of the lock actuator 1272. The engagement feature 1274 may be biased toward or in the extended configuration via a force (e.g., a compression force) applied to the engagement feature 1274 by spring 1273. In some variations, the force may be about 10 psi to about 400 psi, such as about 15 psi to about 300 psi, about 0 psi to about 250 psi, about 25 psi to about 200 psi, about 30 psi to about 150 psi, about 40 psi to about 100 psi, about 50 psi to about 75 psi, greater than or about equal to 20 psi, greater than or about equal to 25 psi, greater than or about equal to 30 psi, greater than or about equal to 35 greater than or about equal to, greater than or about equal to 40 psi, greater than or about equal to 45 psi, greater than or about equal to 50 psi, greater than or about equal to 75 psi, or greater than or about equal to 100 psi (including all ranges and subranges therebetween). The force applied by the spring 1273 may be overcome to releasably engage the engagement feature 1274 with a corresponding engagement feature of the actuator (e.g., engagement feature 1156a of FIG. 11A). For example, a portion of the actuator (e.g., the actuator engagement feature) may be pressed onto (e.g., via an actuator body) the lock engagement feature 1274 to overcome the spring force, transitioning the lock engagement feature 1274 to the receded configuration. When the actuator is subsequently released, the spring force may be re-exerted by spring 1273 such that the lock engagement feature 1274 transitions to the extended configuration and engages the corresponding engagement feature of the actuator (e.g., by extending through a distal aperture of the actuator engagement feature). To release the engagement, the lock actuator 1272 may be configured to receive an opposing force (e.g., a compression force) that is greater than or equal to the force exerted by the spring 1273 to again transition the lock engagement feature 1274 to the receded configuration and release the actuator from the closed configuration. In some variations, actuating the lock actuator 1272 may include translating (e.g., sliding) the lock actuator 1272 vertically along a vertical or longitudinal axis of the body 1271. Further, in some variations, the lock actuator 1272 may include a grip 1276 configured to aid a user in palpating the lock actuator 1272 for actuation. Similarly, in some variations, the lock actuator 1272 may include a surface pattern 1277 in the form of a label or symbol, such as an arrow, to visually indicate to the user a direction of the force configured to be received by the lock actuator 1272.

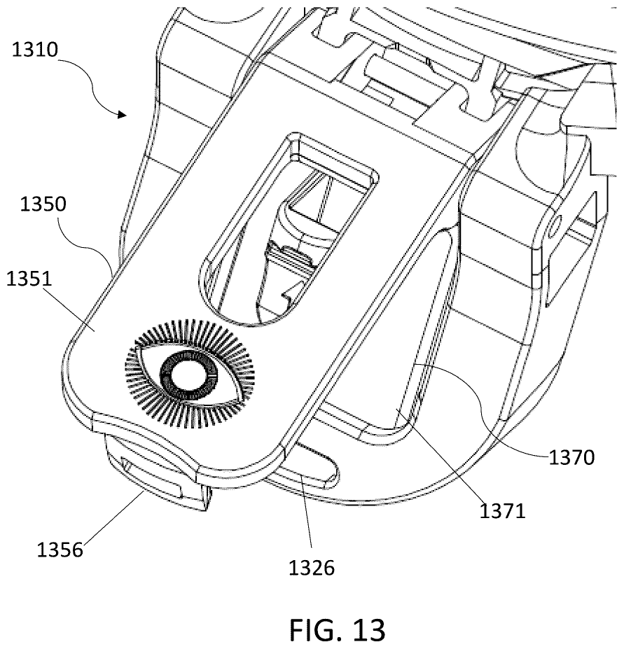

FIG. 13 is a perspective view of a tissue excision device body 1310 (“device body”) having an open configuration of an actuator 1350 relative to the device body 1310 and/or a lock 1370. For example, an actuator body 1351 of the actuator 1350 may have the open configuration relative to a lock body 1371 of the lock 1370 and/or to the device body 1310. Each of the actuator 1350 and the lock 1370 may be coupled to the device body 1310 (e.g., via pins, as explained with reference to FIG. 2). Further, the device body 1310 may include an aperture 1326 extending transversely through the device body 1310. The aperture may be configured to receive a first engagement feature 1356 of the actuator 1350. The first engagement feature 1356 may releasably engage with a second engagement feature (not shown) of the lock 1370 within the space provided by the aperture 1326.

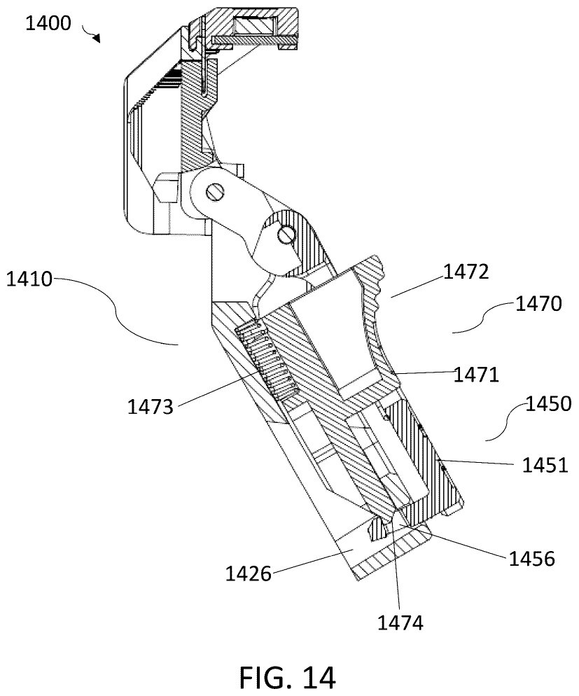

FIG. 14 is a cross-sectional view of a tissue excision device 1400 with a device body 1410 having an actuator 1450 and a lock 1470 coupled thereto. As shown, an actuator body 1451 of an actuator 1450 may be a closed configuration relative to a device body 1410 and a lock body 1471 of the lock 1470. In the closed configuration, a second engagement feature 1474 of the lock 1470 may extend through an aperture 1426 within the device body 1410 due to a force applied to the second engagement feature 1474 (via the lock body 1471) by a spring 1473 within the lock body 1471. The aperture 1426 may be housing a first engagement feature 1456 of the actuator 1450 such that the second engagement feature 1474 also extends at least partially through an aperture within the first engagement feature 1456. Thus, the actuator 1450 may be held in the closed position by the lock 1470. To transition the actuator 1450 to the open configuration, an opposing force that is equal to or greater than the force applied by the spring 1473 may be applied to the lock actuator 1472 to translate the second engagement feature 1474 (via the lock body 1471), removing the second engagement feature 1474 from the first engagement feature 1456.

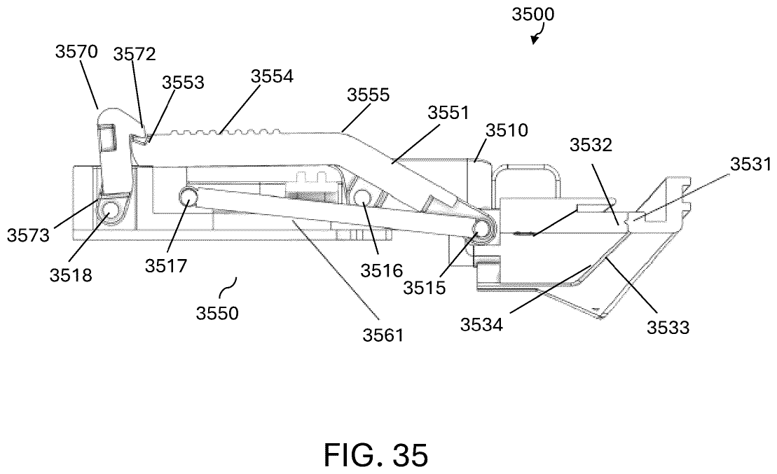

FIGS. 35-38B depict variations of linkage assemblies for use in tissue excision devices. These assemblies may comprise combinations of lever actuators (e.g., bent lever actuators), one or more biasing members (e.g., torsion and tension springs), linkage arms with or without integrated stops, lock features with spring-biased engagement, and various forms of angular stops to define precise and repeatable jaw actuation behavior. Features shown and described herein are compatible with one another and may be implemented in different configurations to accommodate user ergonomics, tissue compression force thresholds, manufacturability, and surgical preferences. The use of integrated engagement surfaces, spring-coupled components, and mechanical travel constraints supports robust and user-intuitive operation. In some variations, the linkage assembly may be preassembled as a module and inserted into a body cavity of the tissue excision device, enabling simplified manufacturing and improved maintenance. Stop members may be interchangeable or switchable between actuator-mounted and linkage arm-mounted configurations, and may be designed for tool-less replacement. These configurations allow for precise and stable control of jaw motion, promote predictable behavior during use, and may be selected based on desired ergonomic, mechanical, or assembly preferences. All of the following variations may be compatible with clamping and cutting functionality described elsewhere herein, and may be further modified to optimize manufacturing efficiency, user interaction, or performance during tissue excision procedures. Each of FIGS. 35-38B is described in more detail below.

FIG. 35 provides a cross-sectional side view of an exemplary tissue excision device 3500 including a linkage assembly 3550 in a closed jaw configuration. An actuator 3551, which may comprise a bent or curved profile including a grip surface 3554 and optional texturing or contouring for tactile feedback, may be pivotally coupled to the body at pivot joint 3515. The bend 3555 in the actuator 3551 may define an angle that facilitates ergonomic actuation while maintaining mechanical advantage during jaw closure. The actuator 3551 may comprise one or more engagement features, such as male engagement member 3572 or female engagement member 3553, to interact with a corresponding feature of the lock 3570, which is also pivotally coupled to the body at pivot joint 3518. These engagement features may comprise interlocking geometries, such as hooks, protrusions, recesses, or complementary surfaces that enable secure coupling and controlled release. When actuated, the actuator 3551 may rotate about pivot 3515, causing linear motion of second jaw 3532 relative to first jaw 3531. In some variations, the actuator 3551 may be coupled to the second jaw 3532 via an intermediate linkage component, thereby creating a compound mechanical advantage. The second jaw 3532 may be pivotally coupled to the actuator via joint 3515 and may include an upper tissue contact surface 3533. The tissue contact surface 3533 may comprise texturing, ridges, or other surface features to enhance tissue gripping capabilities. The second jaw may be biased toward an open position by a first biasing member 3561 (e.g., a tension spring) coupled between the anchor points 3515 and 3517. The biasing member 3561 may be configured to provide sufficient return force to overcome tissue resistance and friction within the linkage assembly. A linkage arm (not shown in this view) may be positioned parallel to or below the actuator and configured to limit motion of the actuator in the open position. In this view, the actuator is shown in a closed configuration, compressing the tissue between the jaws. The lock 3570 may further include a second biasing member 3573 (e.g., a torsion spring) to facilitate its actuation and retraction, such as upon receiving a user input or opposing force. The torsion spring 3573 may have a spring constant selected to provide tactile feedback to the user without requiring excessive force to overcome. The entire linkage assembly 3550 may be held in a collapsed position via actuation of the actuator and engagement of the lock. Additional mechanical stops, detents, or rotational angle limits may be provided within the actuator or linkage components to define travel boundaries for each element. The angle defined by actuator bend 3555 may be between about 20 degrees to about 160 degrees in the closed configuration, or between about 30 degrees to about 90 degrees in variations prioritizing ergonomic actuation force application. The precise angle selection may influence the mechanical advantage achieved during jaw closure, thereby affecting the tissue compression force generated for a given user input force.

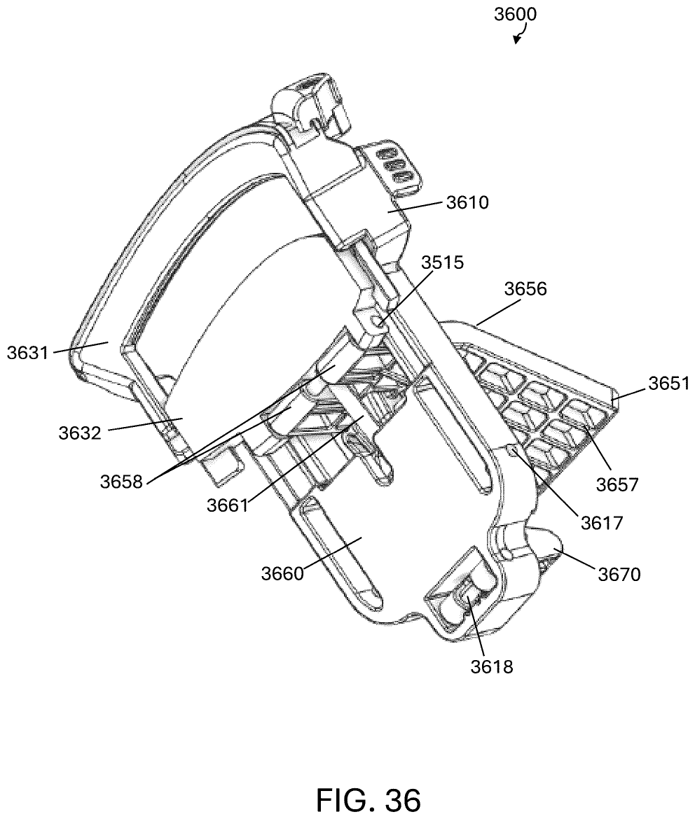

FIG. 36 shows a perspective back view of a tissue excision device 3600 having a linkage assembly in an open or resting configuration. The actuator 3651 may have a top/front surface and an underside/back surface, the latter of which may include contact surface 3658 configured to contact a lower/proximal portion of the second jaw 3632 to limit range of movement. The actuator 3651, second jaw 3632, and first jaw 3631 may be pivotally coupled to the body 3610 via a common joint 3615. This coaxial mounting arrangement may reduce part count and simplify assembly while maintaining precise alignment between components. The lock 3670 may be coupled to actuator 3651 via first and second (e.g., male and female) engagement features 3656 and 3618. These engagement features may comprise complementary geometries designed to create a positive mechanical interlock when engaged. A first biasing member 3661, such as a tension or torsion spring, may be coupled between the actuator and an anchor point on the body 3610 to bias the actuator to an open position. The spring constant of this biasing member may be selected to balance the desire for positive jaw opening against the need to prevent excessive jaw separation velocity. In some variations, actuator movement may be constrained by interaction with the linkage arm (not shown) or a stop member extending from the linkage arm. The actuator 3651 may define a lower boundary of the jaw opening range, while stop member(s), which may be an integrated stop geometry of the linkage arm, may define the upper boundary. In this variation, the lock 3670 may be partially retracted, allowing the actuator 3651 to assume an angled orientation relative to the housing 3610, in which the jaws are held open at a defined distance. The geometric relationship between the actuator 3651, the housing 3610, and the jaws 3631, 3632 may be precisely defined to ensure consistent and repeatable opening behavior.

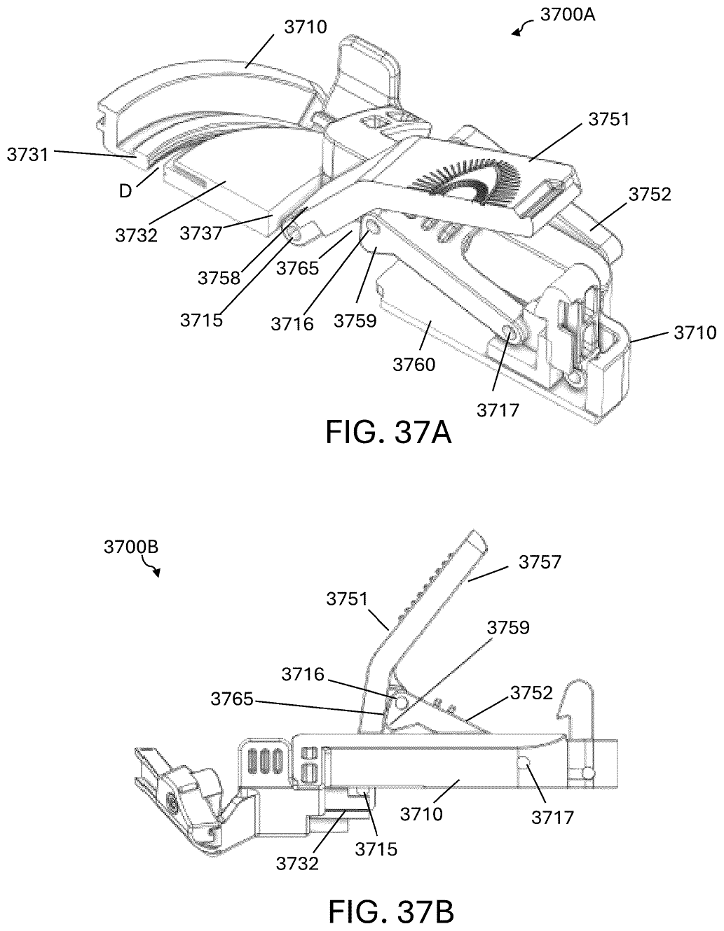

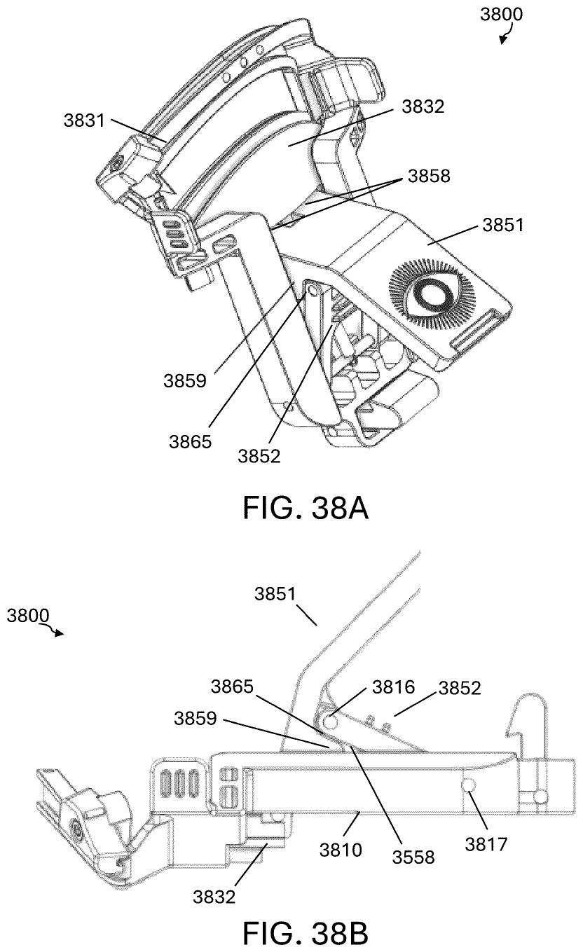

FIGS. 37A-38B illustrate alternative jaw stop configurations for tissue excision devices. Each variation defines a maximum open position of the jaws using a mechanical stop member that engages between the actuator and another device component. Specifically, FIGS. 37A-37B depict a first variation in which the stop member is integrated with the linkage arm and engages the actuator, whereas FIGS. 38A-38B show a second variation in which the stop member is integrated with the actuator and engages the linkage arm. These alternative placements provide different mechanical advantages and design tradeoffs.

FIGS. 37A and 37B illustrate a first jaw stop variation of a tissue excision device 3700A, 3700B in which a stop member is integrated into the linkage arm rather than the actuator. These figures show the tissue excision device in partially and fully open configurations, respectively, and provide visual detail regarding the spatial relationships among the actuator 3751, linkage arm 3752, and second jaw 3732. In this variation, the stop functionality is achieved through an engagement between a protrusion formed on the linkage arm 3752 and a lower surface 3757 of the actuator 3751.

As shown in FIG. 37A (perspective view), the linkage arm 3752 includes a stop member 3759 that may be integrally formed with or separately attached to the arm. The stop member 3759 may include an upper contact surface 3758 that is angled or stepped, and configured to contact the underside 3757 of actuator 3751 when the jaws reach an open limit. In the partially open configuration, the actuator 3751 is rotated upward but the contact surface 3758 of the stop member 3759 is not yet in full parallel contact with the underside 3757, leaving further angular travel available. This configuration may represent an intermediate state between closed and fully open positions. The second jaw 3732 is pivotally coupled to the body 3710 at joint 3715 and may be advanced or retracted by actuation of linkage arm 3752 about pivot 3716. A torsion or compression spring (not shown) may bias the actuator 3751 upward, and a lock mechanism may releasably secure the actuator in a closed configuration.

FIG. 37B shows a corresponding side view of the device 3700B in a fully open configuration. In this position, the stop member 3759 on linkage arm 3752 contacts the underside 3757 of the actuator 3751 at an interface 3765. The contact geometry at interface 3765 may define a zero-degree angle between the mating surfaces, limiting further angular displacement of the actuator and thereby setting a consistent open position of the second jaw 3732 relative to the first jaw 3731. This stop member 3759 may comprise any suitable geometry such as a rectangular block, a curved surface, a rounded protrusion, or a stepped profile. By precisely defining the open angle of the actuator relative to the body, the stop geometry ensures ergonomic operation and prevents hyperextension of the actuator. The device 3700B may further comprise a baseplate 3760 lacking any integrated stop surface, as the open limit is fully defined by interaction between the actuator and linkage arm.

FIGS. 38A and 38B illustrate a second jaw stop variation in which the stop member is integrated into the actuator rather than the linkage arm. FIG. 38A shows a perspective view of tissue excision device 3800 with actuator 3851 having a protruding stop member 3859 extending from an underside surface near its posterior region. The stop member 3859 may be monolithically formed with the actuator or affixed by fasteners, adhesives, or inserts. The actuator 3851 rotates about pivot joint 3816 and drives the second jaw 3832 relative to the first jaw 3831 through coupled motion with linkage arm 3852. In this variation, the stop member 3859 is configured to contact an engagement surface 3858 on the linkage arm 3852. The linkage arm may be biased toward a collapsed or expanded state via biasing member 3861 (e.g., tension spring or torsion spring), and may rotate about pivot joint 3817 formed within the body 3810 of the device.

In FIG. 38B, the actuator 3851 is shown in a fully open configuration. The stop member 3859 protruding from the actuator underside contacts the upper surface 3858 of the linkage arm 3852 at an interface 3865, preventing further upward rotation of the actuator 3851. The contact geometry at the interface may define a zero-degree relative angle between the actuator and the linkage arm at full open, or may define a slight offset (e.g., about 5 degrees to about 20 degrees) to tailor jaw positioning. The actuator-mounted stop member 3859 may comprise any geometry sufficient to engage the linkage arm and reliably halt rotation, such as a curved lip, sloped wedge, tab, or keyed protrusion. As in the first variation, this stop interaction establishes a defined open limit for the second jaw, which may range from about 3 mm to about 25 mm (e.g., about 10 mm) depending on clinical use.

The key distinction between the two stop member variations shown in FIGS. 37A-37B and FIGS. 38A-38B lies in the placement of the stop member and the direction of the mechanical interaction. In the first variation (FIGS. 37A-37B), the stop member 3759 is integrated with the linkage arm 3752 and interfaces with the actuator 3751. In the second variation (FIGS. 38A-38B), the stop member 3859 is integrated with the actuator 3851 and interfaces with the linkage arm 3852. Each variation may offer distinct advantages in terms of manufacturability, mechanical leverage, and force distribution. For example, linkage arm-integrated stop members may allow greater angular travel of the actuator and may be less exposed to user contact, whereas actuator-integrated stop members may enable more compact component packaging and simplify integration with actuator-mounted locking mechanisms. Either variation may be used in isolation or in combination with other stop features such as integrated detents, jaw slots, or baseplate projections.

### Cutting Assembly

The tissue excision devices herein may additionally comprise a cutting assembly configured to excise (e.g., cut) tissue secured by jaws of the tissue excision devices. The cutting assembly may be configured to excise tissue by cutting tissue laterally along a tissue excision device. Generally, the cutting assembly may be moveably (e.g., slidably) coupled to the jaws, such as to a first jaw thereof. In particular, the cutting assembly may have a first position, or initial position, at or proximal to a first end (e.g., a first lateral end) of the jaws and a second, opposite position, or final position, at or proximal to a second, opposite end (e.g., a second lateral end) of the jaws. In some variations, the cutting assembly may be configured to be moved one time from the first position to the second position. Additionally, as explained above, the jaws may comprise a cutting assembly lock configured to prevent additional movement of the cutting assembly (e.g., from the second position back toward the first position) after the cutting assembly is moved one time. Alternatively, in some variations, the cutting assembly may be configured to be exchanged between the first and second positions any suitable number of times.

The cutting assembly may comprise one or more housing portions coupled together to form a housing structure that retains a cutting element. In some variations, the cutting assembly may comprise a first housing portion and a second housing portion that may be coupled to one another via coupling elements such as screws, pins, snap-fit connectors, or other mechanical fastening techniques. This modular approach may facilitate assembly, maintenance, and potential replacement of individual components. The first housing portion may define at least one channel configured to couple to a cutting assembly guide on the first jaw, and may comprise a plurality of contoured surfaces to facilitate ergonomic manipulation of the cutting assembly during a tissue excision procedure. The second housing portion may define a slot or groove configured to receive and securely retain the cutting element, thereby allowing for precise positioning of the cutting edge relative to tissue to be excised.

Generally, the cutting assembly may be moved in at least one direction with respect to the jaws. Briefly considering the tissue excision device 600 of FIGS. 6A and 6B and the XYZ coordinate system shown and described with respect thereto, where the Y-axis may define a central longitudinal axis of the tissue excision device may be a vertical or Y-axis of the device 600, a first direction of movement of the cutting assembly 690 along the jaws 630 may be in the negative direction along the X-axis and/or, due to the curvature of the jaws 630, in the positive direction along the Z-axis and, subsequently, in the negative direction along the Z-axis. Put another way, with respect to the X-axis, the cutting assembly 690 may be moved from a first position to a second position along the X-axis (e.g., from a right side of the device 600 to a left side of the device 600), where the first position has an X-coordinate that is greater than an X-coordinate of the second position. Because the Y-axis may define a central longitudinal axis of the device 600, the first X-coordinate may be positive, and the second X-coordinate, mirrored across the Y-axis, may be negative. With respect to the Z-axis, the cutting assembly 690 may be moved from a first position to a second position along the Z-axis, where the first position has an Z-coordinate that is about equal to a Z-coordinate of the second position, and where an intermediate position of the cutting assembly (e.g., at point I) along the Z-axis has a Z-coordinate that is greater than the Z-coordinates of the first and second positions. Oppositely, a second direction of movement of the cutting assembly 690 along the jaws 630 may be in the positive direction along the X-axis and/or, as for the first direction, in the positive direction along the Z-axis and, subsequently, in the negative direction along the Z-axis. That is, with respect to the X-axis, the cutting assembly 690 may be moved from a first position to a second position along the X-axis (e.g., from a left side of the device 600 to a right side of the device 600), where the first position has an X-coordinate that is less than an X-coordinate of the second position. Again, because the Y-axis may define a central longitudinal axis of the device 600, the first X-coordinate may be negative, and the second X-coordinate, mirrored across the Y-axis, may be positive. When the device 600 is With respect to the Z-axis, the cutting assembly 690 may be moved from a first position to a second position along the Z-axis, where the first position has an Z-coordinate that is about equal to a Z-coordinate of the second position, and where an intermediate position of the cutting assembly (e.g., at point I) along the Z-axis has a Z-coordinate that is greater than the Z-coordinates of the first and second positions.

In general, the cutting assembly may comprise a housing having a handle and/or a contact surface, and a cutting element. The handle and/or contact surface may facilitate manual movement of the cutting assembly while maintaining orientation and grip. The handle and the contact surface may each be configured to be contacted (e.g., grasped, pushed, pulled, touched, etc.) by a user (e.g., using one or more fingers) to move the cutting assembly in the desired direction. In some variations, the handle may be fixed to (e.g., integrally formed with or formed separately and fixedly coupled) the housing. The contact surface may comprise at least one region, such as two or more neighboring regions having similar or different geometries. For instance, the contact surface may comprise a first region having a substantially linear profile and a second region having a curved profile (e.g., concave, convex, or compound curved). In other variations, the contact surface may comprise a plurality of regions of alternating geometries to accommodate different finger positions or hand sizes. The contact surface regions may be configured to receive and/or support one or more fingers of the user while the cutting assembly is being moved via the handle. The transition between neighboring contact surface regions may be gradual and smooth, or may comprise distinct edges that provide tactile feedback to the user regarding finger placement. One or more of the contact surface regions and/or the handle may comprise text, symbols, or other markings that may be printed, etched, embossed, debossed, molded, or otherwise provided thereon. Such markings may serve various purposes, including but not limited to: indicating a direction of intended movement (e.g., arrows, directional symbols), identifying a type of cutting assembly (e.g., “L” or “R” to denote left or right configurations), providing orientation guidance (e.g., “UP” or an orientation arrow), indicating compatible tissue excision devices or anatomical sites (e.g., “eyelid,” “upper,” or “lower”), displaying warnings or precautions (e.g., “single use,” “sterile”), or providing manufacturer information (e.g., brand name, model number, or patent numbers). The texture of the contact surface regions may also vary, with some regions having a smooth finish and others having a textured finish (e.g., ridged, stippled, knurled, or micro-textured) to enhance grip security during use. In some variations, the contact surface may comprise a material or coating different from the remainder of the housing, such as a soft-touch polymer, silicone overlay, or elastomeric insert, to improve tactile feedback and reduce finger fatigue during extended procedures. Generally, the handle and the contact surface may be positioned on opposite sides of the housing, (e.g., on opposite ends of a top surface) of the housing. Alternatively, a cutting assembly may comprise a handle and contact surface oriented on a same housing side (e.g., a front surface thereof).

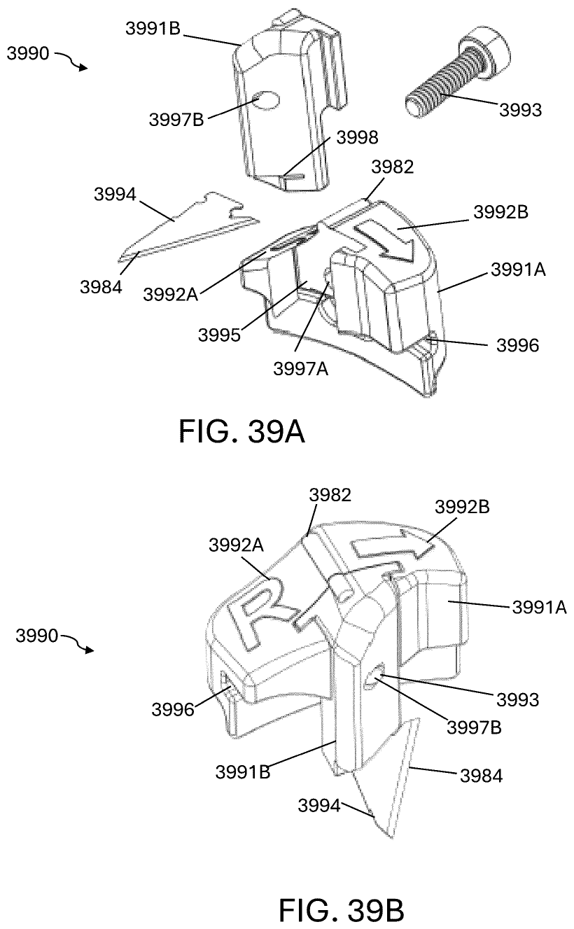

FIGS. 39A and 39B illustrate an exploded view and an assembled view, respectively, of a cutting assembly 3990 comprising first and second housing portions coupled together. The first housing portion 3991A may comprise at least one channel 3996 configured to couple to a cutting assembly guide on a device body (e.g., to sidewalls defining a guide track thereof). The first housing portion 3991A may also comprise a first contact surface region 3992A having contoured portions that may facilitate ergonomic manipulation during use. These contoured regions may be designed to receive user contact and facilitate movement of the cutting assembly 3990 along the cutting guide during use. The first housing portion 3991A may further define a first slot 3995 configured to receive at least part of the second housing portion 3991B therein. The first housing portion 3991A may further comprise a first upper (distal) portion of lumen 3997A for receiving a coupling element 3993. The second housing portion 3991B may comprise a second slot 3998 configured to receive a cutting element 3994 therein (e.g., a blade with at least one cutting edge 3984). The second housing portion 3991B may also comprise a second lower (proximal) portion of lumen 3997B therethrough configured to receive the coupling element 3993. The first housing portion 3991A may have a slot that is sized and shaped to receive the second housing portion 3991B, thereby allowing the second housing portion 3991B to be releasably coupled with the first housing portion 3991A. In some variations, the second housing portion 3991B may be removable from the first housing portion 3991A. The interface/midpoint 3982 between the first and second contact surfaces 3992A/B may serve as a handle that extends laterally or transversely to improve grip during use.

The first and second housing portions 3991A, 3991B may be coupled via a coupling element 3993 (e.g., a screw, pin, etc.). Either or both portions may comprise curved or contoured surfaces (e.g., convex, concave, or curved in an arc). In some variations, the first housing portion 3991A may comprise finger hold regions 3992A to improve grip and facilitate movement of the cutting assembly 3990. Text symbols or other distinguishing markings on the contact surfaces may facilitate distinguishing between device types or cutting directions. The cutting element 3994 may be secured within the second housing portion 3991B via a pin or other suitable coupling feature.

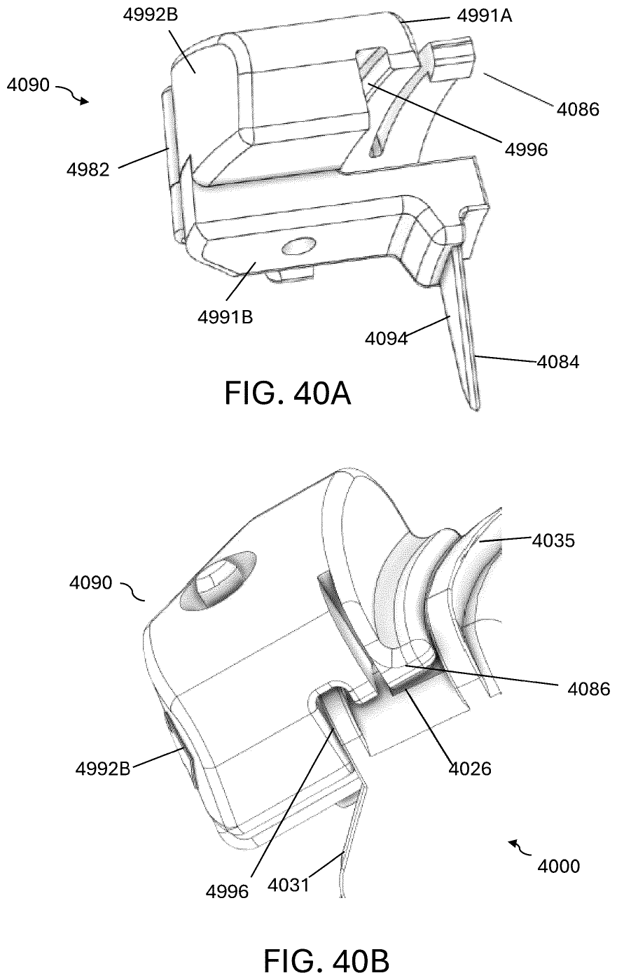

FIGS. 40A and 40B illustrate side views a cutting assembly 4090 having modified housing geometry. FIG. 40A shows a side view wherein the cutting element 4094 may extend transversely from the second housing portion 4091B (e.g., at about a 90 degree angle). This perpendicular orientation may optimize cutting mechanics by providing a direct transfer of force from the user's input to the tissue being excised. The second housing portion 4091B may have a longitudinal axis that is perpendicular to the longitudinal axis of the first housing portion 4091A. This orthogonal configuration may maintain the cutting element 4094 proximal to the clamped jaw interface while the coupled housing portions provide longitudinal bulk that displaces contact surfaces from the clamped jaw interface/cutting element and simultaneously provides sufficient surface area for secure gripping. The perpendicular arrangement of the housing portions may also enhance mechanical stability during the cutting operation by distributing forces across multiple axes, thereby reducing the likelihood of unwanted movement or deflection of the cutting element.

The cutting assembly 4090 (e.g., one or both housing portions) may comprise an engagement feature 4086 configured to mate with a corresponding feature on a cutting assembly guide 4035. The engagement features may be designed to provide both tactile and audible feedback when properly engaged, potentially producing a distinct click or increased resistance at the moment of engagement. This configuration may be useful to prevent the cutting assembly from moving after an excision procedure (e.g., one slide of the cutting assembly from an initial to a final position on the cutting assembly guide) is complete, thereby reducing the risk of accidental tissue damage or incomplete excision. In some variations, the engagement feature 4086 may be positioned on a first side/end of the cutting assembly (e.g., on a lateral end of the first housing portion), and the cutting edge 4084 may face toward that first side/end. This arrangement ensures that as the cutting assembly is advanced, the cutting edge leads the movement, creating a clean, progressive cut through the target tissue.

FIG. 40B depicts a blown up view of a portion of a tissue excision device 4000 comprising a first jaw 4031 having a cutting assembly guide 4035 and a cutting assembly 4090 disposed thereon. The cutting assembly 4090 may engage the guide 4035 via at least one channel 4026 thereof, and the channel 4026 may extend longitudinally through the first housing portion 4091A. The channel-and-guide interface may be precision-manufactured to provide smooth translation with minimal lateral play, thereby ensuring consistent cutting performance throughout the excision procedure. The cutting assembly guide 4035 may comprise a first engagement feature 4026 (e.g., recess, protrusion, or other complementary geometry) configured to mate with a corresponding engagement feature 4086 (e.g., hook, detent, or complementary surface) on cutting assembly 4090. These engagement features may be designed with specific interference tolerances to ensure secure engagement while allowing for intentional disengagement when necessary. In the illustrated embodiment, the engagement features 4026, 4086 are coupled, which may indicate that the device 4000 has been used and is now in a final, locked position that prevents further movement of the cutting element 4094. This locking mechanism may serve as a safety feature that prevents reuse of single-use devices and/or indicates completion of a cutting procedure.

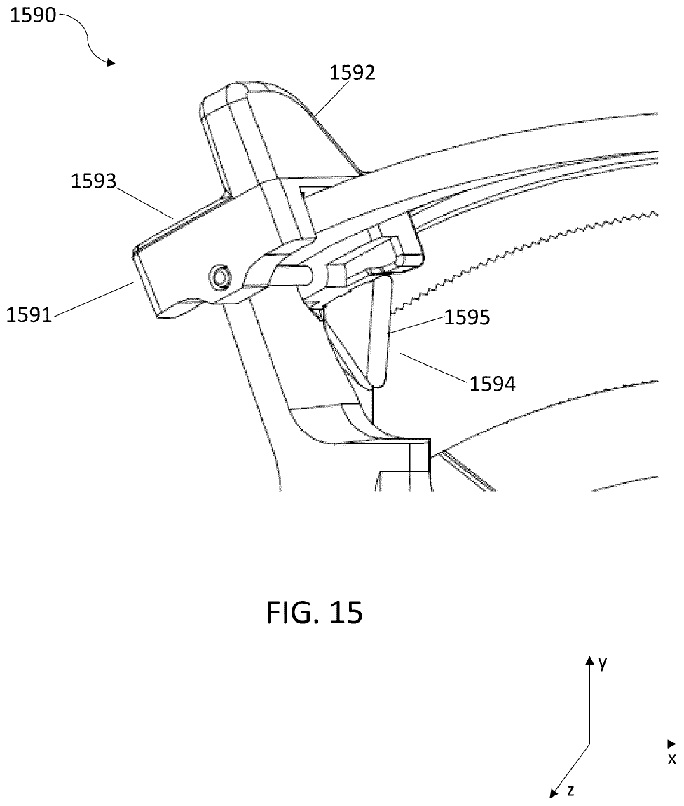

FIG. 15 is a perspective front view of an exemplary cutting assembly 1590 having the components introduced above. More specifically, the cutting assembly 1590 may include a housing 1591 with a handle 1592 positioned thereon and a contact surface 1593 next to the handle 1592. Further, the cutting assembly 1590 may include a cutting element 1594 coupled to the housing 1591 (e.g., to an interior surface thereof). The cutting element 1594 may extend distally from the housing 1591 along a longitudinal axis thereof, which may be a Y-axis of the XYZ coordinate system shown. In some variations, the handle 1592 may extend proximally from the housing 1591 along the longitudinal or Y-axis of the cutting assembly 1590. For example, the handle 1592 may extend proximally from an external surface, such as from a top or proximal surface or end, of the housing 1591. Additionally, or alternatively, in some variations, the handle may extend perpendicularly, relative to the XY-plane shown in FIG. 15, from the housing 1591, such as from a side surface of the housing 1591. In some variations, the handle 1592 and the cutting element 1594 may be aligned relative to a central longitudinal axis of the cutting assembly 1590. In some variations, the handle 1592 and the cutting element 1594 may be offset relative to the central longitudinal axis (e.g., offset along the X-axis shown). Generally, a central longitudinal axis of the handle 1592 may be parallel to a central longitudinal axis of the cutting element 1594.

Furthermore, the cutting assembly may have a configuration that facilitates movement of the cutting assembly toward a given direction along the jaws. For example, a position of the handle on the housing and/or an angle of a cutting edge of the cutting element may be optimized to facilitate movement of the cutting assembly along the jaws of the tissue excision device and through (via the cutting edge of the cutting element) the tissue being excised.

With respect to the handle, in some variations, the handle may be positioned on a first end of the cutting assembly that is proximal to the direction in which the cutting assembly is configured to be moved. For example, a cutting assembly configured to be moved only in a first or second direction along an axis (e.g., an X-axis, as in FIGS. 6A, 6B, and FIG. 15) may include a handle that is positioned toward or on a corresponding first or second end (e.g., first or second lateral end along the X-axis shown in FIGS. 6A, 6B, and FIG. 15) of the cutting assembly. This configuration of the handle may allow a user to simply push the cutting assembly toward the intended direction of movement thereof using, for example, a single finger, while maximizing a surface area of the contact surface next to the handle to support the finger(s) of the user during tissue excision. A surface area of the contact surface may be about 10 mm² to about 200 mm², such as about 25 mm² to about 175 mm², about 50 mm² to about 150 mm², about 75 mm² to about 125 mm², or about 100 mm² to about 110 mm² (e.g., less than about 75 mm², about 75 mm2, about 80 mm², about 85 mm², about 90 mm², about 95 mm², about 100 mm², about 105 mm², about 110 mm², or greater than about 110 mm²). Moreover, a maximum length (e.g., along a longitudinal axis of a tissue excision device) of the handle may be about 0.5 mm to about 20 mm such as about 1 mm to about 15 mm, about 2 mm to about 10 mm, about 3 mm to about 8 mm, about 4 mm to about 6 mm, or about 5 mm (including all ranges and subranges therebetween).

Further, when the handle is positioned on a top surface (e.g., a surface facing the positive or proximal direction of a longitudinal or vertical axis of the cutting assembly or tissue excision device) of the housing, as opposed to a side surface (e.g., a front side surface) thereof, a rotational force applied to the handle (via the user) to move the cutting assembly during tissue excision may be limited or prevented. This may help to maintain the cutting element in its intended orientation relative to the tissue being excised.

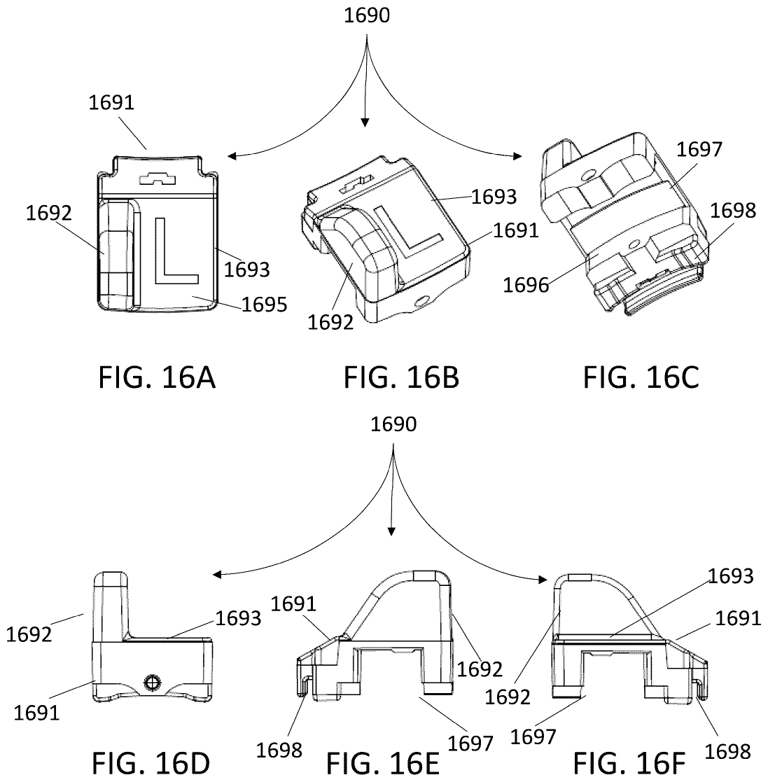

FIGS. 16A-16F show various views of a cutting assembly housing 1691 (“housing”) having a first configuration. Specifically, FIG. 16A is a top view of the housing 1691, FIG. 16B is another top view of the housing 1691, FIG. 16C is a bottom view of the housing 1691, FIG. 16D is a first (e.g., front) side view of the housing 1691, FIG. 16E is a second (e.g., left) side view of the housing 1691, and FIG. 16F is a third (e.g., right) side view of the housing 1691. The first configuration shown in FIGS. 16A-16F may be one in which a handle 1692 of the housing 1691 is positioned toward a first direction of movement of the cutting assembly 1690. In some variations, the first configuration may be considered a “left” configuration of a cutting assembly because, when a user aligns a tissue excision device having the first configuration on a tissue excision site, a handle 1692 may be on a left side of the housing from the perspective of the user. In particular, as shown, the handle 1692 may be on a left side of a first (e.g., top) exterior surface 1695 of the housing 1691. A contact surface 1693 may be positioned next to the handle 1602, also on the first exterior surface 1695. Referring to FIG. 16C, the housing 1691 may have a first (e.g., back) interior surface 1696 where a cutting element (not shown) may be coupled to the housing 1691 (e.g., via any suitable fastener, such as, for example, a pin). Additionally, referring to FIG. 16C and FIGS. 16E and 16F, the housing 1691 may include first and second recesses 1697, 1698 where the housing 1691 may be moveably attached to jaws of a tissue excision device, such as on a cutting assembly guide of a first jaw of the tissue excision device. The cutting element coupled to the housing 1691 may have a cutting edge that faces toward the first (e.g., “left”) direction. Thus, the user may apply a force to the cutting assembly 1690 to move in the first direction, and the cutting element may excise tissue via the cutting edge that faces the first direction. In some variations, the housing 1691 may include a surface pattern in the form of text or an image (e.g., a symbol) on one or more surfaces thereof, such as the letter “L” shown on the first exterior surface 1695 FIGS. 16A and 16B to indicate the configuration of the cutting assembly 1690. In some variations, a tissue excision device having the cutting assembly 1690 may be configured for use on one or more anatomical areas of a patient, such as only on a first (e.g., right) upper eyelid of the patient.

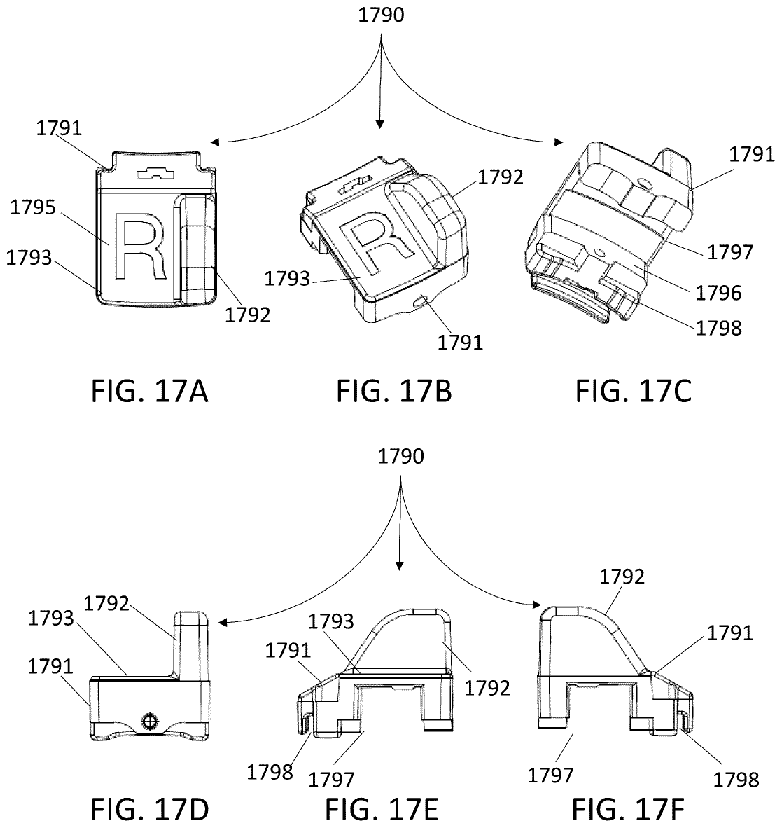

Oppositely, FIGS. 17A-17F show various views of a cutting assembly housing 1791 (“housing”) having a second configuration. Specifically, FIG. 17A is a top view of the housing 1791, FIG. 17B is another top view of the housing 1791, FIG. 17C is a bottom view of the housing 1791, FIG. 17D is a first (e.g., front) side view of the housing 1791, FIG. 17E is a second (e.g., left) side view of the housing 1791, and FIG. 17F is a third (e.g., right) side view of the housing 1791. The first configuration shown in FIGS. 17A-17F may be one in which a handle 1792 of the housing 1791 is positioned toward a first direction of movement of the cutting assembly 1790. In some variations, the first configuration may be considered a “right” configuration of a cutting assembly because, when a user aligns a tissue excision device having the second configuration on a tissue excision site, a handle 1792 may be on a right side of the housing from the perspective of the user. In particular, as shown, the handle 1792 may be on a right side of a first (e.g., top) exterior surface 1795 of the housing 1791. A contact surface 1793 may be positioned next to the handle 1792, also on the first exterior surface 1795. Referring to FIG. 17C, the housing 1791 may have a first (e.g., back) interior surface 1796 where a cutting element (not shown) may be coupled to the housing 1791 (e.g., via a pin). Additionally, referring to FIG. 17C and FIGS. 17E and 17F, the housing 1791 may include first and second recesses 1797, 1798 where the housing 1791 may be moveably attached to jaws of a tissue excision device, such as on a cutting assembly guide of a first jaw of the tissue excision device. The cutting element coupled to the housing 1791 may have a cutting edge that faces toward the second (e.g., “right”) direction. Thus, the user may apply a force to the cutting assembly 1790 to move in the second direction, and the cutting element may excise tissue via the cutting edge that faces the second direction. In some variations, the housing 1791 may include a surface pattern in the form of text or an image (e.g., a symbol) on one or more surfaces thereof, such as the letter “R” shown on the first exterior surface 1795 of FIGS. 17A and 17B to indicate the configuration of the cutting assembly 1790. In some variations, a tissue excision device having the cutting assembly 1790 may be configured for use on one or more anatomical areas of a patient, such as only on a second (e.g., left) upper eyelid of the patient.

In some variations, the cutting element of the cutting assembly may include a bevel (e.g., a single or double bevel) and, as described above, a cutting edge of the cutting element may be a first edge of the bevel that faces a same direction in which the cutting assembly is configured to be moved. This configuration, as opposed to a cutting element configuration including two opposing cutting edges on a bevel of the same size, may allow for an angle of the cutting edge to be maximized, which may improve a cut quality of the tissue excision (e.g., by reducing a force necessary to cut through the tissue). That is, a cutting edge having a larger angle with respect to a central longitudinal or vertical axis of the cutting assembly may improve cutting performance of the cutting element. Thus, a cutting element having only a single cutting edge may only require that the angle of the cutting edge, and not the angle of the second, non-functional edge, be maximized relative to the central longitudinal axis. Accordingly, a size of the cutting element with the single cutting edge at a desired angle may be smaller than a size of a cutting element with two cutting edges, each at the same desired angle. This may be preferable to configure a tissue cutting device having relatively smaller dimensions of components that interact with the cutting element (e.g., cutting assembly guide, slot configured to receive the cutting element, etc.) and to minimize a gap between the cutting element and a front side of the device to maximize an amount of excess tissue excised using the tissue excising device. Furthermore, a larger cutting element angle may reduce a cutting force required to cut through the tissue as compared to a smaller cutting element angle. Generally, an angle of the cutting element, relative to a central longitudinal axis of the cutting assembly, may be about 10 degrees to about 80 degrees, such as about 30 degrees to about 45 degrees, about 20 degrees, about 25 degrees, about 30 degrees, about 35 degrees, about 40 degrees, about 45 degrees, or about 50 degrees. An average thickness of the cutting edge may be less than an average thickness of a remainder of the cutting element. In some variations, a thickness of the cutting edge may vary. For example, a thickness of the cutting edge may decrease (e.g., linearly or substantially so) from a first (e.g., inner) end of the cutting edge to a second (e.g., outer) edge thereof. In some variations, a maximum thickness of the cutting edge may be about 15 μm to about 1,000 μm, such as about 25 μm to about 500 μm, about 50 μm to about 250 μm, about 75 μm to about 200 μm, or about 100 μm to about 150 mm (including all ranges and subranges therebetween). Oppositely, a minimum thickness of the cutting edge may be about 10 μm to about 500 μm, such as about 15 μm to about 250 μm, about 25 μm to about 200 μm, about 50 μm to about 150 μm, or about 75 μm to about 100 μm (including all ranges and subranges therebetween). Moreover, a maximum length of the cutting edge may be about 0.5 mm to about 20 mm such as about 1 mm to about 15 mm, about 2 mm to about 10 mm, about 3 mm to about 8 mm, about 4 mm to about 6 mm, or about 5 mm (including all ranges and subranges therebetween). Further, a maximum length of the cutting element about 1 mm to about 30 mm such as about 2 mm to about 25 mm, about 3 mm to about 20 mm, about 4 mm to about 15 mm, about 5 mm to about 12.5 mm, about 7.5 mm to about 10 mm, or about 10 mm (including all ranges and subranges therebetween).

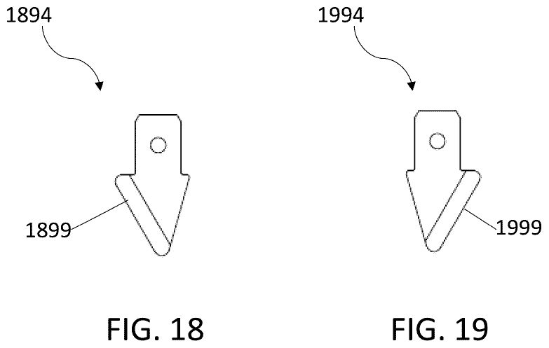

FIG. 18 is a front view of an exemplary cutting element 1894 in a first configuration, in which a cutting edge 1899 thereof may face a same first direction (e.g., leftward, from a user's perspective) as a handle of a cutting assembly housing configured to couple with the cutting element 1894. For example, the cutting element 1894 may be used with the cutting assembly housing 1691 depicted in FIGS. 16A-16F. Oppositely, FIG. 19 is a front view of an exemplary cutting element 1994 in a second configuration, in which a cutting edge 1999 thereof may face a same first direction (e.g., rightward, from a user's perspective) as a handle of a cutting assembly housing configured to couple with the cutting element 1994. For example, the cutting element 1994 may be used with the cutting assembly housing 1791 depicted in FIGS. 17A-17F. One or of the cutting elements 1894, 1994 may have a single or double bevel. Further, in some variations, one or both of the cutting elements may include a second cutting edge opposite the cutting edge 1899, 1999, which may be a first cutting edge.

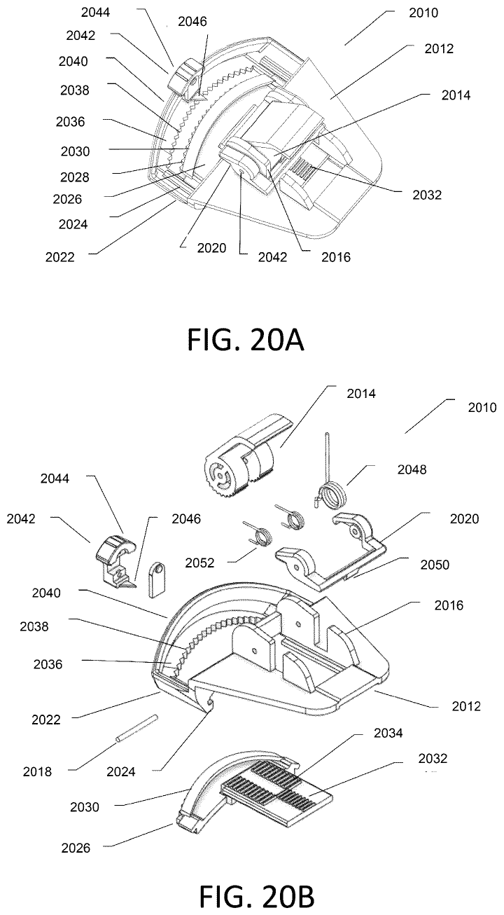

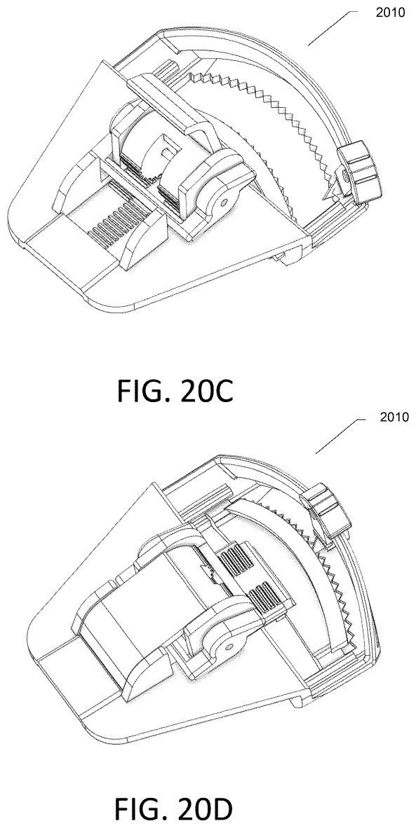

In some variations, the cutting assemblies herein may include a handle that projects outward from a first side surface, such as a front side surface, of a housing of the cutting assembly. Such an exemplary configuration is shown in FIGS. 20A-23D, which illustrate various tissue excision devices having a handle 2044, 2194, 22154, or 23244. In some variations, such the handle may be aligned with a central longitudinal axis of the cutting assembly or cutting assembly. Alternatively, in some variations, the handle extending from the first side surface of the cutting element assembly may be offset from a center of the cutting element assembly, such as offset along a transverse axis of the cutting element assembly.

### 1.2. Alternative Tissue Excision Device Configurations

Various exemplary alternative configurations of the tissue excision devices herein (and aspects thereof) will be described below. While the following configurations may be described with reference to FIGS. 20A-23B, it should be understood that one or more of the features described below may be incorporated into and/or replaced with one or more corresponding features of tissue excision devices described above and shown in FIGS. 1A-19.

FIGS. 20A and 20B are perspective and exploded views, respectively, of an exemplary alternative tissue excision device 2010 that is configured to be actuated via a rachet system. The tissue excision device 2010 (“device”) may be a skin removal and wound closure system that may generally include a body 2012, a pinion gear 2014, a gear mount 2016, a gear axle 2018, and a ratchet assembly 2020. The body 2012 may include a frame 2022 and a guide rail 2024. A second jaw 2026 may be movable (e.g., slidable) within the guide rail 2024. The second jaw 2026 may include second serrations 2028, cutting element guard 2030, ratchet teeth 2032, and gear rack 2034. The frame 2022 may include a first jaw 2036 (e.g., fixed thereto) with first serrations 2038. Further, the frame 2022 may include a cutting guide 40 upon which a cutting assembly 2042 may be moved. The cutting assembly 2042 may include a handle 2044 and a cutting element 2046. The pinion gear 2014 may engage with gear rack 2034 such that rotation of the pinion gear 2014 results in linear movement of the second jaw 2026 along the guide rail 24. Rotation of the pinion gear 2014 may bring the second jaw 2026 into a closed clamping position where the second jaw 2026 may be proximal to the first jaw 2036. An example of the closed position of the device 2010 is shown in FIG. 20D. Oppositely, rotation of the pinion gear 2014 in the reverse direction may place the first and second jaws 2026, 2036 in an open position where the second jaw 2026 is distal to the first jaw 2036. An example of the open position of the device 2010 is shown in FIG. 20C. Further, the ratchet assembly 2020 may include a catch 2050 and a ratchet spring set 2052. The ratchet assembly 2020 may allow movement of the second jaw 2026 in the clamping direction (e.g., proximally toward the first jaw 2036) and may prevent reverse motion until released. A torsion spring 48 may bias the pinion gear 2014 to hold the first and second jaws 2036, 2026 in the open position.

In practice, the first jaw 2036 and the second jaw 2026 may be set in the open position while the cutting assembly 2042 may be positioned in a pre-cutting or initial position at a first end of the cutting guide 2040. Excess tissue may be positioned between the first and second jaws 2036, 2026, for example, using a grasper as described herein. The pinion gear 14 may be rotated to bring the second jaw 2026 into contact with the excess tissue. Using pre-drawn markings (e.g., drawn by a surgeon or other medical professional) as a guide, the tissue may be positioned such that the markings are aligned with front surfaces of the first and second jaw members 2036, 2026. The pinion gear 2014 may then be rotated to advance the second jaw 2026 such that the tissue is clamped between the two jaws at a pressure to achieve hemostasis (e.g., about 10 psi to about 50 psi, such as about 30 psi). The ratchet assembly 2020 may interact with ratchet teeth 2032 to hold the second jaw 2026 in place relative to the first jaw 2036 until the ratchet assembly 2020 is released. When the excess tissue is properly clamped, the cutting assembly 2042 may be manually slid along the cutting guide 2040 to excise the tissue that sits above the front surfaces of the first and second jaw members 2036, 2026. Once cutting is complete and the excess tissue is removed, the rachet assembly 2020 may be released. When the ratchet assembly 2020 is in a released position, the torsion spring 2048 may exert torque on pinion gear 2014 such that the second jaw 2026 returns to an open position and the device may be withdrawn from the patient.

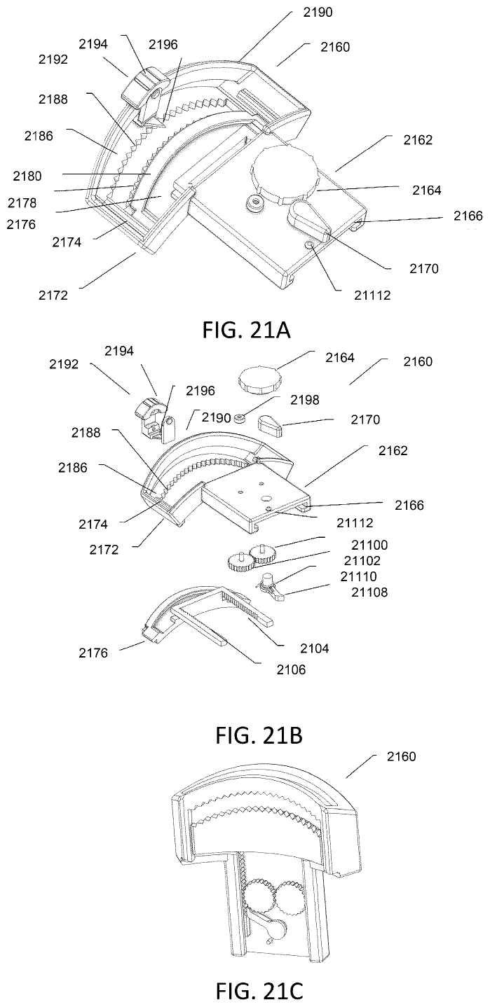

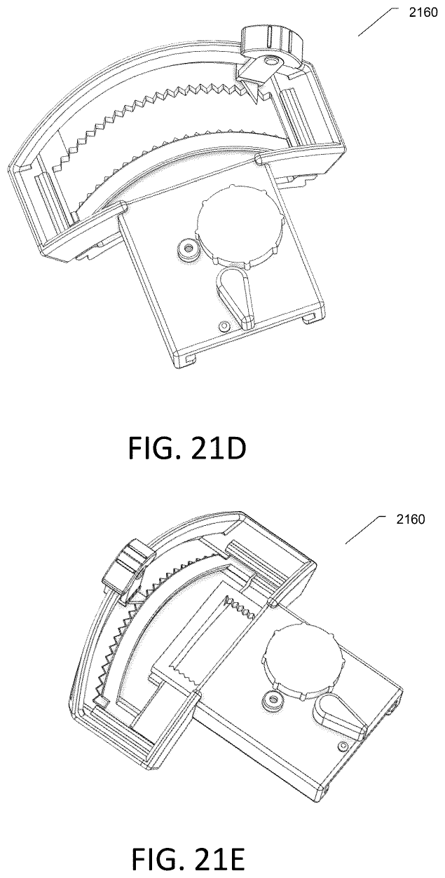

Another exemplary alternative tissue excision device, device 2160, is depicted in FIGS. 21A-21E. FIG. 21A is a perspective view of the device 2160, which may be a tissue removal and wound closure device that is configured to be actuated via a rotational system. The device 2160 may include a body 2162, a drive knob 2164, a channel 2166, and a release lever 2170. The body 2162 may include a frame 2172 and a guide rail 2174. The second jaw 2176 may be movable (e.g., slidable) within the guide rail 2174. Additionally, the second jaw 2176 may include second serrations 2178, and cutting element guard 2180. The frame 2172 may feature a first jaw 2186 (e.g., integrally formed with the frame) and first serrations 2188. Moreover, the frame 2172 may include a cutting guide 2190 upon which the cutting assembly 2192 may be moved. The cutting assembly 2192 may include a handle 2194 and a cutting element 2196. Further, the device 2160 may be configured such that rotation of the drive knob 2164 results in linear movement of second jaw 2176 along the guide rail 2174.

Additionally, FIG. 21B depicts an exploded view of the device 2160. As shown, the device 2160 may include the drive knob 2164 which may be rotated with respect to the body 2162 and may engage with a first gear 21100 to drive the first gear 21100. The first gear 21100 may additionally engage with a second gear 21102 and a first (e.g., right) rack 21104. The second gear 21102 may engage with the first gear 21100 and a second (e.g., left) rack 21106. A nut 2198 may couple the second gear 21102 to the body 2162. Further, the right and left racks 21104, 21106 may be attached to the second jaw 2176. Rotation of the drive knob 2164 with respect to the body 2162 may result in advancement or retraction of the second jaw 2176 with respect to the first jaw 2186. In some variations, rotation of the drive knob 2164 in a first direction with respect to the body 2162 may bring the second jaw 2176 into a closed clamping position where the second jaw 2176 is proximal to the first jaw 2186. An example of the device 2160 in the closed position is shown in FIG. 21E. Oppositely, rotation of drive knob 2164 in the reverse direction may position the first and second jaw members 2186, 2176 in an open position. An example of the device 2160 in the open position is shown in FIG. 21D. Moreover, the second gear 21102 may interface with a pawl 21108. The pawl 21108 may comprise a spring 21110 and may couple to release lever 2170. The pawl 21108 and the spring 21110 may combine to allow movement of the second jaw 2176 in the clamping direction while preventing reverse movement of the second jaw 2176 (unless released). Further, the body 2170 may include a protrusion 21112 configured to hold the release lever 2170 and pawl 21108 in an unlocked position. The spring 21110 may bias the pawl 21108 to catch on teeth of the second gear 21102 to prevent retraction of second jaw 2176. Pivoting the release lever 70 beyond the protrusion 21112 may release the pawl 21108 from the second gear 21102, enabling retraction of the second jaw 2176 via rotation of the drive knob 2164 in a second, opposite direction with respect to the body 2162.

In practice, the first jaw 2186 and the second jaw 2176 may be set in the open position while the cutting assembly 2192 may be positioned in a pre-cutting position at a first end of the cutting guide 2190. Excess tissue may be positioned between the first jaw 2186 and the second jaw 2176. The drive knob 2164 may be rotated to bring the second jaw 2176 into contact with the tissue. Using pre-drawn markings as a guide, the tissue may be positioned such that the markings are aligned with front surfaces of the first and second jaws 2186, 2176. The drive knob 2164 may then be rotated in a first direction to advance the second jaw 2176 such that the tissue is clamped between the two jaws at a pressure to induce hemostasis within the tissue. The pawl 21108 may interact with the second gear 21102 to hold the second jaw 2176 in place relative to the first jaw 2186. Once the excess tissue is clamped, the cutting assembly 2192 may be manually moved (e.g., slid) along the cutting guide 2190 to excise the excess tissue that sits above front surfaces of the first and second jaw members 2186, 2176. Once the excess tissue is removed, the first jaw 2186 and the second jaw 2176 may be spread apart by pivoting the release latch 2170 to its unlocked position and subsequently rotating the drive knob 2164 in a second, opposite direction.

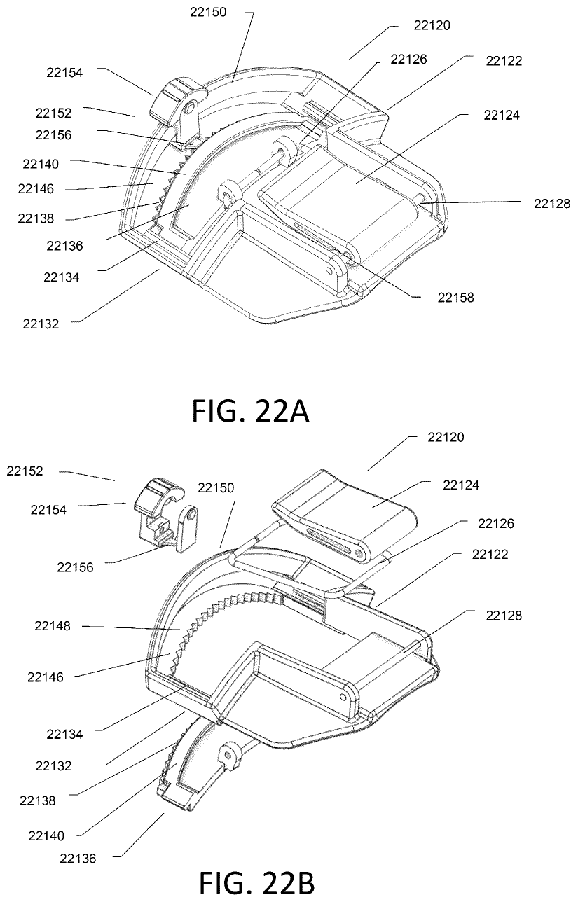

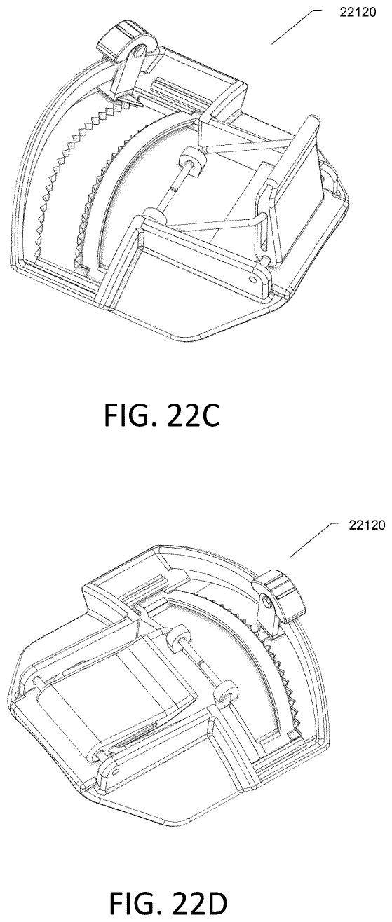

Yet another exemplary alternative tissue excision device, device 22120, is depicted in FIGS. 22A-22D. FIGS. 22A and 22B are perspective and exploded views, respectively, of the device 22150, which may be a tissue removal and/or wound closure device that is configured to be actuated via a pivot system. The device 22120 may include a body 22122, a handle 22124, a keeper 22126, and an axle 22128. The handle 22124 may be rotatable with respect to the body 22122 via the axle 22128. Additionally, the keeper 22126 may be rotatable and translatable with respect to the handle 22124 via a slot 22158. The keeper 22126 may be rotatably coupled to a second jaw 22136, which may be movable within a guide rail 22134. The second jaw 22136 may include second serrations 22138 and a cutting element guard 22140. Moreover, the frame 22132 may feature a first jaw 22146 having first serrations 22148. The frame 22132 may additionally include a cutting guide 22150 upon which a cutting assembly 22152 may be moved. The cutting assembly 22152 may include a handle 22154 and a cutting element 22156.

In some variations, rotation of the handle 22124 in a first direction (e.g., toward a front surface of the device 22120) may move the keeper 22126 in the first direction and toward a first (e.g., proximal) end of the device 22120. This action may advance the second jaw 22136 along the guide rail 22134. Rotating the handle 22124 such that a longitudinal axis thereof is parallel to a longitudinal axis of the body 22122 may move the second jaw 22136 into a closed clamping position where the second jaw 22136 is proximal to the first jaw 22146. An example of the device 22120 in the closed position is depicted in FIG. 22D. In the closed position, the handle 22124 and keeper 22126 may be rotated past parallel such that the angulation and compressive forces that result lock the first and second jaws 22146, 22136 in the closed position. Further, rotation of handle 22124 in a second, opposite direction (e.g., away from a front surface of the device 22120) may release the compressive forces and move the second jaw 22136 away from the first jaw 22146 (e.g., toward the open position). An example of the device 22120 in the open position is depicted in FIG. 22C.

In practice, the first jaw 22146 and the second jaw 22136 may be set in the open position while the cutting assembly 22152 may be positioned in a pre-cutting position at a first end of the cutting guide 22150. Excess tissue may be positioned between first jaw 22146 and second jaw 22136. The handle 22124 may be pivoted in the first direction, toward a front surface of the device 22120, to bring second jaw 22136 into contact with the tissue. Using pre-drawn markings as a guide, the tissue may be positioned such that the markings are aligned with the front surfaces of the first and second jaws 22146, 22136. Then, the handle 22124 may be fully pivoted to clamp the tissue between the first and second jaws 22146, 22136 at a pressure to achieve hemostasis. Once the excess tissue is clamped, the cutting assembly 152 may be manually moved along the cutting guide 22150 to excise the excess tissue that sits on the front surfaces of the first and second jaw members 22146, 22136. Next, the handle 22124 may be rotated in the second direction, away from the front surface of the device 22120, to open the first and second jaws 22146, 22136 and device 22120 may be removed from the surgical field to prepare for wound closure.

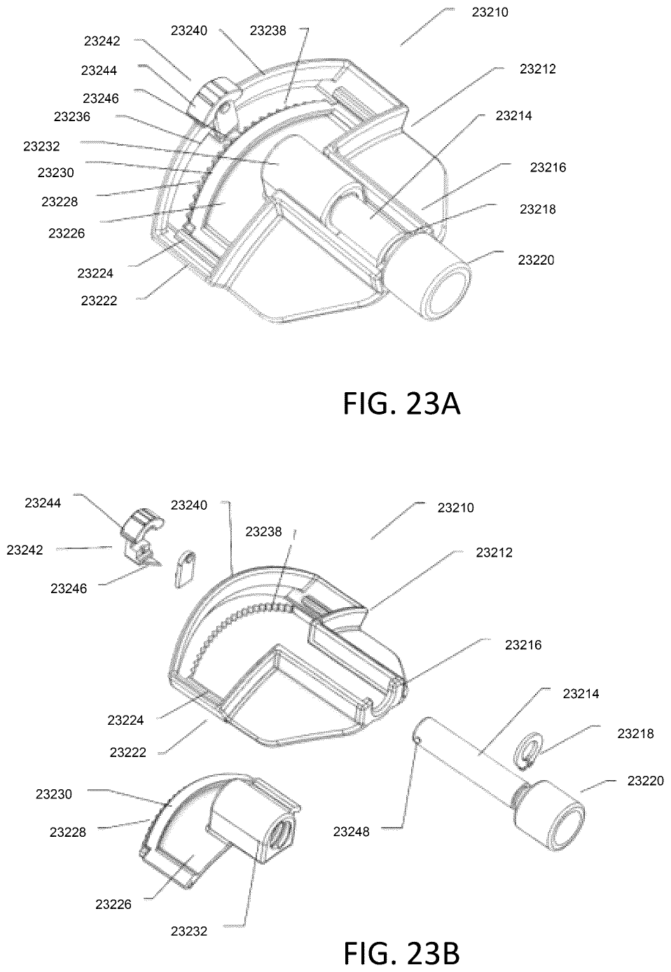

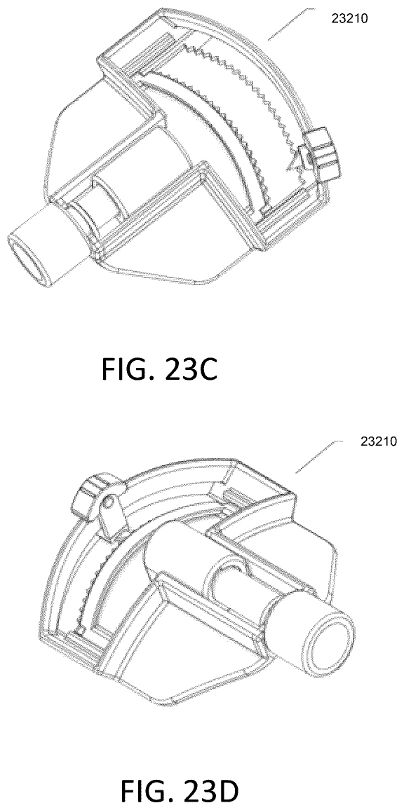

Still another exemplary alternative tissue excision device, device 23210, is depicted in FIGS. 23A-23D. FIGS. 23A and 23B are perspective and exploded views, respectively, of the device 23210, which may be a tissue removal and wound closure device that is configured to be actuated via a rachet system a screw system. The device 23210 may include a body 23212, a lead screw 23214, a screw mount 23216, a snap ring 23218, and a lead screw knob 23220. The body 23212 may feature a frame 23222 and a guide rail 23224. A second jaw 23226 may be movable within the guide rail 23224. Additionally, the second jaw 23226 may include second serrations 23228, a cutting element guard 23230, and a threaded nut 23232. The frame 23222 may include a first jaw 23236 having second serrations 23238. Further, the frame 23222 may include a cutting guide 23240 upon which the cutting assembly 23242 may be moved. The cutting assembly 23242 may include a handle 23244 and a cutting element 23246. The lead screw 23214 may include a pin set 23248 to engage with threaded nut 23232. The lead screw 23214 may be rotatably coupled to the body 23212. Further, the screw mount 23216 and the snap ring 23218 may be configured to prevent linear movement of lead screw 23214. In some variations, rotation of a lead screw knob 23220 may cause the second jaw 23226 to advance and/or retract linearly (relative to the first jaw 23236). For example, rotation of the lead screw knob 23220 may advance second jaw 23226 proximally toward the first jaw 23236, and toward a closed position. An example of a closed position of the device 23210 is shown in FIG. 23D. Oppositely, rotation of the lead screw knob 23220 in a second, opposite direction may retract the second jaw 23226 distally away from the first jaw 23236, toward an open position. An example of an open position of the device 23210 is shown in FIG. 23C. In some variations, a screw pitch of the threaded nut 23232 may be configured to provide a clamping force in the closed position that induces hemostasis and prevents slippage of the tissue within the first and second jaws 23236, 23226 (e.g., a force of about 10 psi to about 50 psi, such as about 30 psi).

In practice, the first jaw 23236 and the second jaw 23226 may be in the open position while the cutting assembly 23242 is positioned in a pre-cutting position at a first end of the cutting guide 23240. Excess tissue may be positioned between the first jaw 23236 and the second jaw 23226. The lead screw 23214 may be rotated in a first direction to bring the second jaw 23226 into contact with the tissue. Using pre-drawn markings as a guide, the tissue may be positioned such that the markings are aligned with the front surfaces of the first and second jaw members 23236, 23226. Next, the lead screw knob 23214 may be further rotated in the first direction to advance second jaw 23226 such that the tissue is clamped between the first and second jaws 23236, 23226 at a pressure configured to induce hemostasis of the tissue. Once the tissue is clamped, the cutting assembly 23242 may be manually moved along the cutting guide 23240 to excise the tissue that sits on the front surfaces of the first and second jaw members 23236, 23226. Next, the lead screw knob 23220 may be rotated in a second, opposite direction to retract second jaw 23226 away from the first jaw 23236. The device 23210 may then then be withdrawn from the patient.

Yet another exemplary alternative tissue excision device may incorporate a guillotine-type cutting mechanism that differs from the previously described configurations. This guillotine configuration may employ a cutting element that moves linearly in a vertical plane to cut tissue in a single action. That is, the guillotine mechanism may enable simultaneous tissue excision across an entire width of the clamped tissue seam. In some variations, the body of this device variation may be fabricated from metal rather than polymer materials to provide enhanced structural rigidity and durability. In this configuration, the first and second jaws may first capture and secure the tissue, after which the cutting element may be descended or advanced through the secured tissue. The cutting element may be moved along a longitudinal axis of the device body. This guillotine mechanism may thus enable simultaneous tissue excision across the entire width of tissue clamped between the jaws. Additionally, the linear cutting motion may reduce the force required from the user while enhancing excision precision.

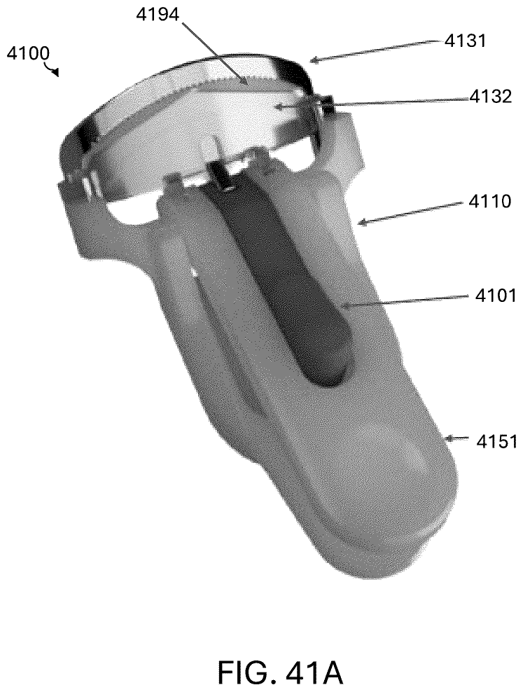

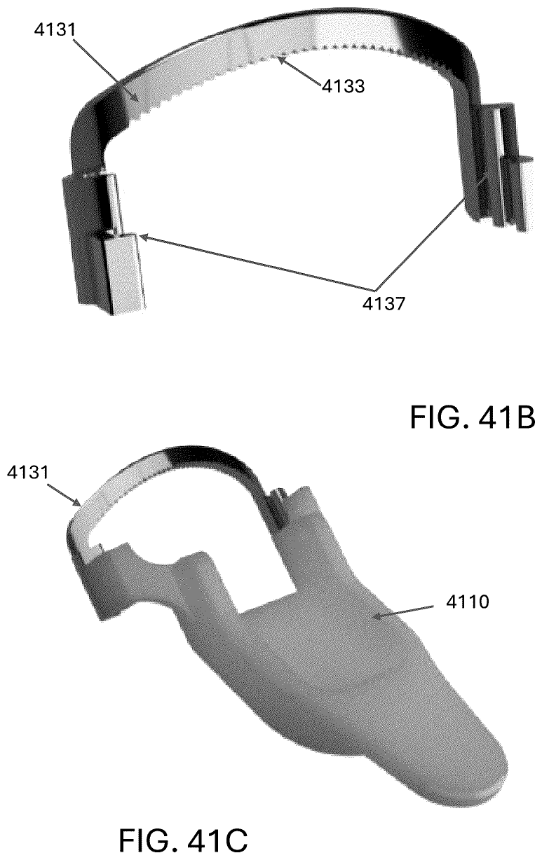

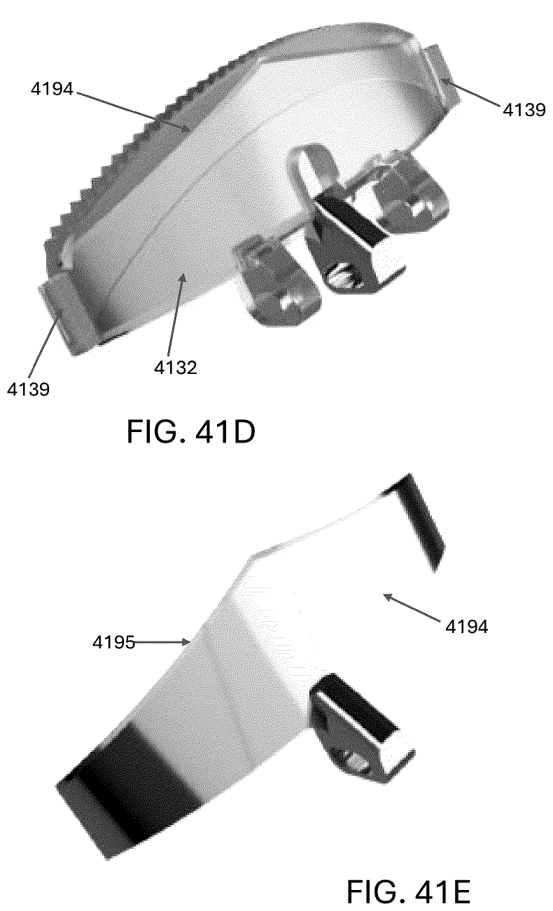

FIGS. 41A-41E depict an exemplary tissue excision device 4100 having a guillotine-type cutting mechanism. As shown in FIG. 41A, the device 4100 may comprise a body 4110 that may have reduced width and/or increased length compared to other devices described herein (e.g., devices 200A/B and 2800A/B). The device 4100 may further comprise an upper jaw 4131 and a lower jaw 4132. In some variations, one or both of the upper and lower jaws 4131, 4132 may be formed of metal, which may provide enhanced mechanical advantage for clamping and may enable higher pressures against the jaws compared to polymer-based devices.

As shown in FIG. 41B, the upper jaw 4131 may comprise a tissue contact surface 4133 and coupling guides 4137 configured to allow movement of the lower jaw 4132 relative to the upper jaw 4131. The upper jaw 4131 may be integrated with the body 4110 or may be coupled (e.g., overmolded) to the body via machining or injection molding, as illustrated in FIG. 41C. Further, FIG. 41D shows that the lower jaw 4132 may comprise coupling features 4139 configured to fit within lumens or channels defined by the coupling guides 4137. The upper and lower jaws 4131, 4132 may comprise serrations or other surface texture as shown, or alternatively may have be smooth.

Unlike previously described devices where the cutting assembly slides along a guide track to excise tissue, this guillotine configuration employs a cutting element 4194 that moves linearly in a vertical plane to cut tissue in a single action. The cutting element 4194 may be inset into the lower jaw 4132 and movable relative thereto. As shown in FIG. 41E, the cutting element 4194 may comprise a cutting edge 4195, such as a blade. In some variations, the cutting element 4194 may be curved. For example, the cutting element 4194 may comprise a chevron shape to help minimize vertical travel required for cutting tissue. At least a portion of the cutting element 4194 may protrude from the lower jaw 4132 (e.g., extending distally toward the upper jaw) during the cutting operation. The cutting element 4194 may be thin-such as about 0.004 inches in thickness—to provide enhance cutting performance.

Furthermore, the device 4100 may comprise a first actuator (e.g., lever) 4151 configured to move one or both of the jaws relative to each other (e.g., the second jaw relative to the first, as also described with respect to devices 200A/B and 2800A/B). Additionally, the device 4100 may comprise a second actuator (e.g., lever) 4101 configured to move the cutting element 4194. In some variations, pivoting movement of the second actuator 4101 may be translated to linear movement of the cutting element 4194 along a longitudinal axis of the device 4100. In some variations, the second actuator 4101 may comprise a lock operably coupled thereto to prevent actuation (e.g., pushing) thereof until tissue is clamped between the first and second jaws. That is, the first actuator 4151 may be used before the second actuator 4101 to clamp tissue, during which the second actuator 4101 may be locked or unactuatable. Then, the second actuator 4101 may become actuatable once tissue is clamped (when the first actuator 4151 may be locked to maintain the clamping) to move the cutting element 4194 and excise the tissue.

Additional details and other suitable tissue excision devices and aspects thereof, such as the body, the jaws, the actuator, and the lock, for use with the systems and methods herein are provided in U.S. patent application Ser. No. 18/140,984, filed Apr. 28, 2023, titled “Wound Creation for Excess Skin Removal and Closure Systems and Methods”, the contents of which are hereby incorporated by reference herein in their entirety.

### 1.3. Graspers

The systems herein may generally include a grasper, such as forceps or another suitable grasping tool, configured to position tissue within an opening of the tissue excision devices herein during tissue excision. The graspers herein may be elongate, manually controlled devices manufactured from any suitable material, such as, for example, metal (e.g., stainless steel, such as medical grade steel) or plastic. In some variations, the graspers herein may be used to interact with tissue to align surgical markings on a tissue excision site relative to markings on a tissue excision device being used to excise tissue of the tissue excision site. This positioning may enable the tissue excision device to excise the tissue in a preferred shape, and may facilitate symmetric excisions of two or more related tissues, such as tissue of both of a first (e.g., right) and second (e.g., left) upper eyelid of a patient.

In one example, the graspers herein may be used (e.g., by a surgeon or other medical professional) to position tissue relative to a tissue excision device for blepharoplasty (i.e., upper eyelid tissue excision). For example, when the issue excision device for blepharoplasty is in an open configuration, tissue of a tissue excision site of a patient may be positioned between the first and second jaws thereof. The graspers herein may be used to facilitate the movement of the tissue into an opening between the first and second jaws. In particular, the graspers herein may be configured to grip upper eyelid tissue of the patient and pull the tissue away from the patient, and into and through the opening.

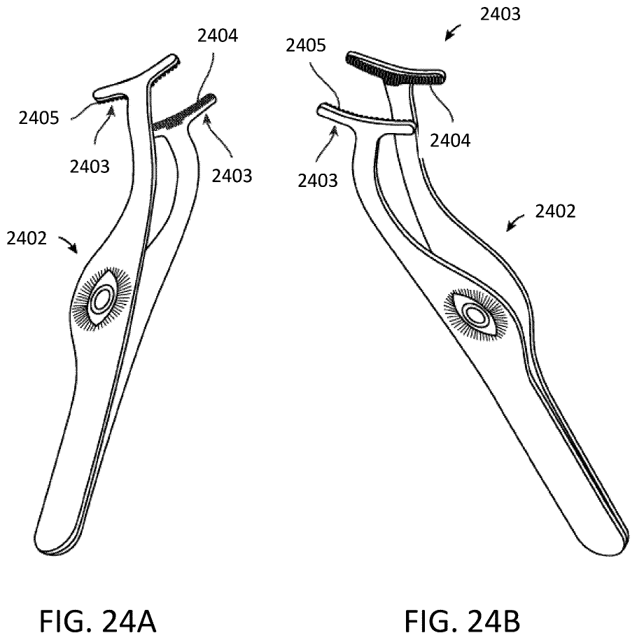

FIGS. 24A and 24B illustrate aspects of a grasper 2402 in an open position or configuration. The grasper 2402 may be actuated from the open position to a closed position, where the distal ends 2403 may be moved toward each other (e.g., via a hand of a user) such that they are touching and/or holding tissue therebetween. The grasper 2402 may include inner surfaces 2404 of the distal ends 2403 that are configured to grasp tissue therebetween. The distal ends 2503 may include various curvatures, widths, and lengths to optimize tissue grasping during a tissue excision procedure. For example, when the grasper 2400 is configured to be used with a tissue excision device for excising tissue on a cheek, neck, underarm, or similarly sized anatomical area of a patient, the distal ends 2503 may include have a first surface area, and when the grasper 2400 is configured to be used with a tissue excision device for excising tissue on an eyeball, lower eyelid, upper eyelid, or similarly sized anatomical area of a patient, the distal ends 2503 may include have a second, smaller surface area. In some variations, the distal ends 2403 may be curved to conform to the intended anatomical area for manipulation.

Moreover, in some variations, the grasper 2402 may include one or more microneedles 2405 at one or both of the distal ends 2403 thereof. In use, the microneedles 2405 may operate to sensitize the tissue to local topical anesthetics and/or to manipulate the tissue.

Additional details and other suitable graspers and aspects thereof for use with the systems and methods herein are provided in U.S. patent application Ser. No. 18/140,984, filed Apr. 28, 2023, titled “Wound Creation for Excess Skin Removal and Closure Systems and Methods”, the contents of which were previously incorporated by reference herein.

### 2. Kits

A tissue excision system including one or more tissue excision devices and/or one or more graspers may be packaged together as a single kit, or may be packaged separately and sold together or separately for use together or separately. In some variations, the kits my further include one or more additional surgical tools, such as one or more of a scalpel, a tissue disruption tool, a cauterizer, sutures, and/or the like. Additionally, or alternatively, in some variations, the kits herein may further include one or more of a local anesthetic, such as lidocaine, and an adhesive, such as a tissue adhesive like cyanoacrylate. One or more units of one or both of the local anesthetic and the adhesive may be included in a single kit.

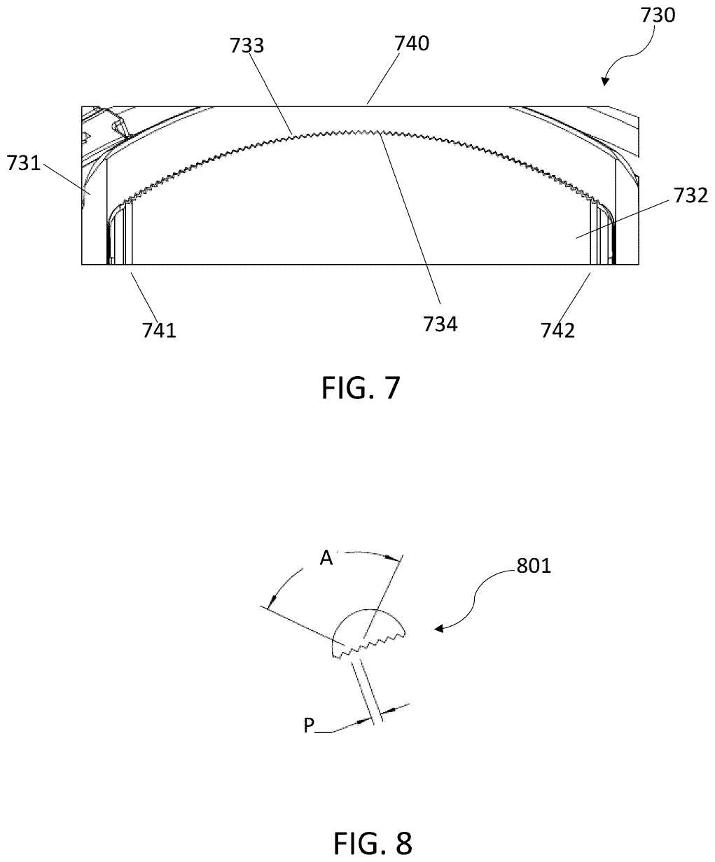

In some variations, the kits may include tissue excision devices having different jaw configurations, such as devices comprising various tongue-and-groove engagement feature arrangements on tissue contact surfaces as described with reference to FIGS. 31A-31B and/or devices comprising serrations along the tissue contact surfaces, as described with reference to FIGS. 7-8. These varying tissue engagement features may provide surgeons with options that optimize tissue compression, hemostasis, and excision precision based on specific anatomical requirements and tissue characteristics. The kits may also include devices with various linkage assembly configurations as shown in FIGS. 35-38B to accommodate different user preferences for actuator ergonomics and jaw motion characteristics, thereby enabling selection of devices with mechanical advantages and actuation forces tailored to individual surgical technique and hand strength. Additionally, kits may include a plurality of devices, some having engagement features for preventing movement of the cutting assembly pre-and/or post-procedure (a “blade lock”), and others lacking these engagement features. In some variations, the devices with blade locks may be configured for single use to ensure sterility and cutting element sharpness, while the devices without blade locks may be configured to be used multiple times with appropriate sterilization between procedures.

Further, the kits herein may include instructions for use (IFU) of the tissue excision device(s) and/or the grasper(s) therein. For example, the IFU may guide a user, such as a surgeon or other medical professional, through performing one or more procedures (e.g., blepharoplasty) using one or more of the tissue excision devices herein.

In some variations, the kits and/or delivery devices described herein may be placed in specialized packaging. The packaging may be designed to protect the systems or devices therein. For example, it may be desirable for the packaging to maintain a tissue excision device therein in an open configuration to prevent a cutting element of the tissue excision device from rubbing against other elements or surfaces of the tissue excision device (which may dull a cutting edge thereof and/or distort the cutting element from its intended position).

In one exemplary variation, the packaging may include a tray having a first recess with a shape generally corresponding to a shape of a first tissue excision device. In some variations, the kits herein may include a plurality of tissue excision devices and/or graspers. For example, a kit may include 2, 3, 4, 5, or than 5 tissue excision devices configured for use on one or more anatomical areas of a patient. As another example, the packaging may include a tray having the first recess and a second recess with a shape generally corresponding to a shape of a second tissue excision device, where the first tissue excision device may be configured for use on a first (e.g., right) upper eyelid, and the second tissue excision device may be configured for use on a second, different (e.g., left) upper eyelid. The first and second tissue excision devices may be configured to be used simultaneously and/or in succession during tissue excision. For example, tissue from the first and second tissue excision sites may be positioned within the jaws of both of the first and second tissue excision devices simultaneously to provide accurate and symmetric relative excisions within the first and second tissue excision sites.

### 3. Methods

Described herein are also methods for excising tissue, including removing excess tissue from a tissue excision site and sealing an excision wound within the tissue excision site. The methods herein may utilize any one of the variations of the tissue excision devices and/or graspers described herein. It should be appreciated that a variety of tissue excision devices and/or graspers with selected differences in dimensions and designs may be provided, and that a trained user may select a device that is most suited for the size and location of the tissue being excised.

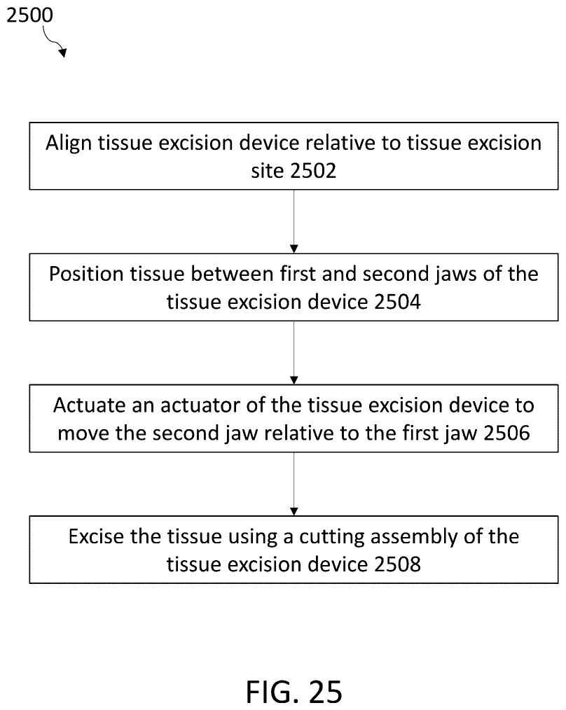

The methods may generally be double-handed, single-user controlled methods that are minimally invasive, e.g., they are tailored for reducing or eliminating a need for cautery and/or sutures during tissue excision, which as previously mentioned, may be advantageous over conventional approaches. For example, eliminating a need for cautery and/or sutures may simplify the procedure for a user (e.g., surgeon or other medical professional) by reducing a number of steps to perform the excision. As another example, eliminating cautery and/or suturing may improve post-operative healing and reduce the recovery period for a patient by reducing an amount/type of trauma applied to the patient during the tissue excision. However, use of cautery, sutures, and/or other suitable surgical techniques may be contemplated in some instances and thus, are not excluded here. The methods herein may be used for medical treatment and/or diagnosis, such as for removing skin lesions from a patient, and/or for cosmetic treatment, such as for removing excess or unwanted tissue. In some variations, the methods herein may be used to excise tissue on one or more anatomical locations of a patient, such as one or more of an upper eyelid, a lower eyelid, an eyeball, an eyebrow, a cheek, a jaw, an underarm, and a neck of a patient. Further, one or more additional treatment modalities may be used with the methods herein, including medication, incisional surgery, laser surgery, cryosurgery, other forms of surgery, and combinations thereof. Exemplary methods will be described in further detail below with reference to FIGS. 25-27.

Turning to FIG. 25, a flow chart of an exemplary method 2500 for excising tissue is depicted. A first step of the method 2500 may include aligning a tissue excision device relative to a tissue excision site of a patient 2502. During the step 2502, the tissue excision device may be in an open configuration defined by a first separation between first and second jaws of the tissue excision device. The first separation may be a dimension (e.g., length) of an opening between the first and second jaws. The step 2502 may include aligning the opening relative to the tissue excision site.

In some variations, the tissue excision device may have one or more transparent portions, such as transparent first and second jaws and/or a transparent body that allow a user to view the tissue excision site through the tissue excision device. For example, the step 2502 may include aligning the tissue excision device relative to the tissue excision site through a transparent first jaw of the device. Additionally, or alternatively, the tissue excision device may comprise one or more markings to aid in symmetrically aligning the tissue excision device on the tissue excision site, such as the alignment markings 3314 described with reference to FIG. 33. Accordingly, the step 2502 may include using one or more markings of the tissue excision device, such as one or more markings on the first jaw thereof, to align the tissue excision device relative to the tissue excision site. In some variations, prior to the step 2502, a local anesthetic may be applied to or injected into the tissue excision site.

A second step of the method 2500 may include positioning tissue between the first and second jaws of the tissue excision device 2504. In some variations, a grasper may be used to grasp and pull the tissue through the opening of the tissue excision device. Next, the method 2500 may include actuating an actuator of the tissue excision device to move the second jaw relative to the first jaw 2506. In some variations, moving the second jaw relative to the first jaw may include reducing a dimension (e.g., a length) of the opening therebetween. For example, the step 2506 may include closing the first and second jaws using the actuator. The actuator may be configured to move the second jaw proximally along a longitudinal axis of the tissue excision device such that the second jaw is translated to a position that is proximal to the first jaw.

In some variations utilizing the linkage assemblies described with reference to FIGS. 35-38B, the actuating step may comprise engaging an actuator to pivot about a body-mounted joint, thereby transferring force through the linkage assembly to the second jaw. The specific actuator configuration may determine the mechanical advantage and ergonomics of the actuation process. For example, devices with bent lever actuators as shown in FIG. 35 may provide different force profiles during jaw closure than those with alternative linkage arrangements.

In some variations, the step 2506 may include clamping the tissue between the first and second jaws. In some variations, the clamping may be achieved and/or aided by engaging the actuator with a lock configured to maintain a position of the actuator, and thus maintain a configuration (e.g., a closed configuration) of the tissue excision device (e.g., of the jaws). The step 2506 may be repeated any suitable number of times to ensure that a desired portion of tissue of the tissue excision site is appropriately clamped within the first and second jaws. In general, a surgeon or medical professional may attempt one or more times to clamp the tissue along surgical markings made on the tissue excision site until a desired position of the device relative to the tissue excision site is achieved. For example, the tissue excision device may be reopened, repositioned, and reclosed on the tissue excision site any suitable number of times.

Finally, the method 2500 may include excising the tissue using a cutting assembly, such as a cutting assembly of the tissue excision device 2508 or a separate cutting tool. During the step 2508, the tissue excision device may be in the closed configuration defined by a second, smaller separation between the first and second jaws of the tissue excision device. In some variations, the step 2508 may include manually moving the cutting assembly from a first position on the tissue excision device to a second position on the tissue excision device.

For devices incorporating the cutting assemblies shown in FIGS. 39A-40B, this may involve grasping the cutting assembly housing at the contact surface regions and sliding the assembly along the cutting assembly guide of the first jaw. Depending on the specific cutting assembly design selected, the cutting motion may be guided by the first and second guide rails/tracks of the cutting assembly guide, while the engagement features help maintain proper positioning throughout the cutting stroke. In some variations, upon reaching the final position, engagement features such as those shown in FIG. 40B (elements 4026, 4086) may engage to prevent inadvertent return movement of the cutting assembly.

In some variations, the first and second positions may be pre-cutting or initial and post-cutting or final positions of the cutting assembly on the tissue excision device. In some variations, the step 2508 may include moving the cutting assembly one time, from the first position to the second position on the tissue excision device. Alternatively, in some variations, the step 2508 may include moving the cutting assembly back and forth along the tissue excision device any suitable number of times (e.g., two, three, four, or more) until a desired cut has been made in the tissue.

The method 2500 may further include removing the excised tissue from the surgical area, such as via the grasper. Similarly, the method 2500 may further include removing the tissue excision device from the surgical area. This may involve disengaging the actuator from the lock to transition the tissue excision device (e.g., the jaws thereof) back to the open configuration and remove the tissue excision device from the tissue excision site. As is explained herein throughout, the tissue excision device may be configured to promote self-sealing of the excision wound. Accordingly, the wound may be partially (e.g., at least partially) or fully sealed, due to cohesive forces within the wound caused by a clamping force and/or surface pattern of the tissue excision device, upon removal of the tissue excision device from the tissue excision site. In some variations, the wound may be further sealed using an adhesive, such as a tissue adhesive. The adhesive may be applied to close the wound before and/or after the tissue excision device is removed from the tissue excision site. Generally, and advantageously, the wound may be sealed without using sutures or cautery.

In some variations, one or more steps of the method 2500 may be optional. For example, in some variations, the method 2500 may be a method for sealing tissue (e.g., without excising tissue), and the step 2508 may be optional. That is, the method 2500 may be used to seal tissue via the actuating step 2506, which may result in a wounded tissue or tissue of a previously excised tissue excision site to at least partially self-seal (e.g., due to a clamping force applied thereto during the actuating 2506). Similarly, in some variations, one or more steps of the method 2500 may be repeated any suitable number of times. For example, one or more of the steps 2502, 2504, and 2506 of the method 2500 may be repeated prior to the excising step 2508 to seal or reseal a wound in the tissue excision site caused by the step 2508. In some variations, the tissue excision device may be actuated again (as in step 2506) to move the second jaw relative to (e.g., away from) the first jaw, and actuated yet again to move the second jaw relative to (e.g., toward) the first jaw to provide another clamping force to the tissue excision site and promote self-sealing of the wound thereon. In some variations, between these two re-actuating steps, the tissue excision device may additionally be realigned relative to the tissue excision site (as in step 2502) to adjust where the following clamping force may be applied.

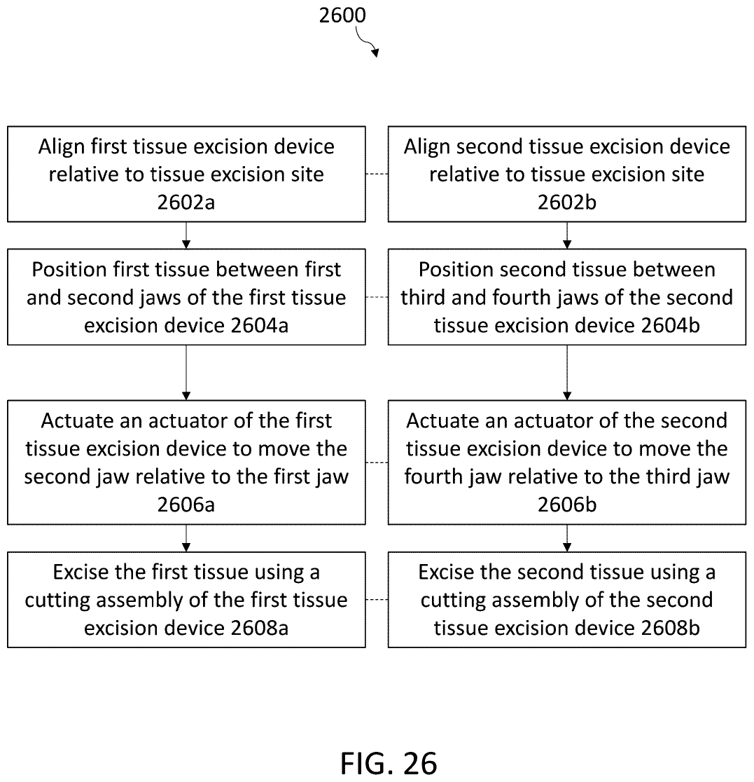

In some variations, one or more steps of the method 2500 may be repeated sequentially or simultaneously using a plurality of tissue excision devices, such as using two or more tissue excision devices. FIG. 26 is a flow diagram of an exemplary method 2600 for excising tissue from first and second tissue excision sites using corresponding first and second tissue excision devices. The method 2600 may be substantially the same as the method 2500 in that it may include the steps of: aligning first and second tissue excision devices relative to first and second tissue excision sites, respectively 2602a,b; positioning first and second tissue between first and second jaws and third and fourth jaws of the first and second tissue excision devices, respectively 2604a,b; actuating an actuator of each of the first and second tissue devices to move each of the second and fourth jaws relative to each of the first and third jaws, respectively 2606a,b; and excising the first and second tissue using a cutting assembly of each of the first and second tissue excision devices, respectively 2608a,b. However, the steps 2602a-2608a may be performed on only the first tissue of the first tissue excision site using the first tissue excision device (having the first and second jaws), while the steps 2602b-2608b may be performed on only the second tissue of the second tissue excision site using the second tissue excision device (having the third and fourth jaws).

In some variations, the first and second tissue excision sites may be on corresponding first and second anatomical locations of the patient. For example, the first and second tissue excision sites may be on first and second upper or lower eyelids, first and second eyeballs, first and second sides of a jaw, first and second underarms, first and second (or more) portions (e.g., sides) of a neck, or first and second cheeks of a patient. The first and second tissue excision devices may be configured for use on only the first or second tissue excision site, respectively.

In some variations, the first tissue excision device may comprise a cutting assembly, such as those described with reference to FIGS. 16A-16F and 39A-40B, configured to be actuated and to excise tissue in a first direction (e.g., from right to left, from a user's point of view), and the second tissue excision device may comprise a cutting assembly, such as those described with reference to FIGS. 17A-17F and 39A-40B, configured to be actuated and to excise tissue in a second, opposite direction (e.g., from left to right, from a user's point of view). The housing and cutting element configuration for these paired devices may be mirror images, with contact surfaces appropriately positioned for ease of use in either a left-handed or right-handed orientation. Accordingly, the step 2608a may include moving the cutting assembly of the first tissue excision device in the first direction (e.g., from a first position toward the second direction to a second position toward the first direction). Oppositely, the step 2608b may include moving the cutting assembly of the second tissue excision device in the second, opposite direction (e.g., from a first position toward the first direction to a second position toward the second direction). In some variations, the method 2600 may include aligning the first tissue excision device relative to the second (e.g., after and/or at the same time as one of the steps 2062a,b), or vice versa, to improve symmetry between excisions resulting from the steps 2608a,b.

While each step 2602a,b-2608a,b may be completed simultaneously, it should be understood that the steps 2602a,b-2608a,b need not all be completed simultaneously. For example, one or more surgeons or other medical professionals may complete steps 2602a-2606a in immediate succession, followed by steps 2602b-2606b in succession, followed by steps 2608a and 2608b. Alternatively, steps 2602b-2606b may be completed before steps 2602a-2606a, and/or step 2608b may be completed before step 2608a. Moreover, in some variations, all of steps 2602a-2608a of the method 2600 may be completed prior to or following completion of all of steps 2602b-2608b. Additionally, the steps 2602a-2608a and/or 2602b-2608b may include one or more optional steps and/or one or more repeated steps, as explained above with reference to the method 2500 of FIG. 25. For example, one or both of the steps 2608a,b may be optional, and/or one or more of the steps 2602a,b-2606a,b may be repeated any suitable number of times.

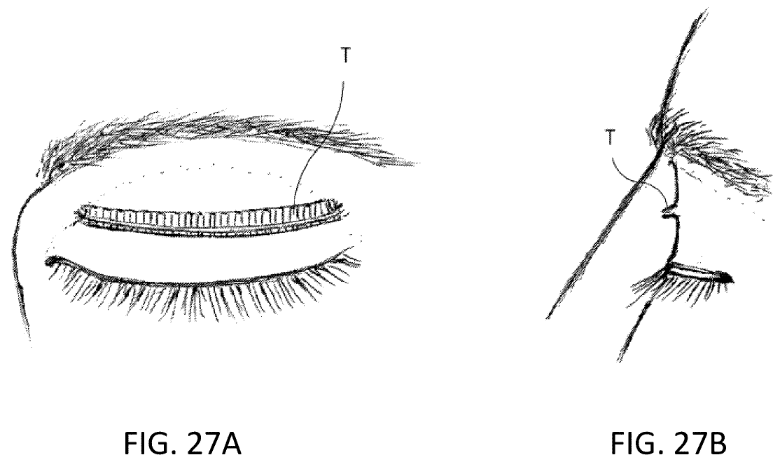

Further, FIGS. 27A and 27B depict a configuration of (at least partially) self-sealed tissue T, such as upper eyelid tissue, after a tissue excision device used to excise the excess tissue thereof is removed from the upper eyelid area. As shown, due to the clamping force applied by the tissue excision device to the tissue T, the tissue T may be pressed against itself along an entire length of the excision site and may protrude from a skin surface of the patient, forming a clamped tissue seam. Thus, FIGS. 27A and 27B illustrate a configuration of the tissue T following step 2508 of the method 2500 and steps 2608a,b of the method 2600.

The acts performed as part of the methods herein may be ordered in any suitable way. Accordingly, various methods may be constructed in which acts are performed in an order different than illustrated, which may include performing some acts simultaneously, even though shown as sequential acts in illustrative examples.

While certain variations are described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive variations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive variations described herein. It is, therefore, to be understood that the foregoing variations are presented by way of example only and that, within the scope of the appended claims and equivalents thereto; inventive variations may be practiced otherwise than as specifically described and claimed. Inventive variations of the present disclosure are directed to each individual feature and/or method described herein. In addition, any combination of two or more such features and/or methods, if such features and/or methods are not mutually inconsistent, is included within the inventive scope of the present disclosure.

All references cited are herein incorporated by reference in their entirety.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

While embodiments of the present invention have been shown and described herein, those skilled in the art will understand that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A device for excising tissue, comprising:
a body;
a first curved member coupled to the body and comprising a first tissue contact surface, the first tissue contact surface comprising a first radius of curvature (ROC);
a second curved member movable relative to the first curved member and comprising a second tissue contact surface comprising a second, smaller ROC;
an actuator coupled to the body and configured to decrease a distance between the first and second curved members; and
a cutting assembly configured to excise the tissue.

2. The device of claim 1, wherein the first ROC is about 30 mm to about 40 mm and the second ROC is about 25 mm to about 35 mm.

3. The device of claim 1, wherein the first ROC is about 1 to about 1.5 times greater than the second ROC.

4. The device of claim 1, wherein, when the first and second curved members are in a closed configuration, a gap is formed between both of a first end and a second end of each of the first and second tissue contact surfaces.

5. The device of claim 1, wherein the second curved member further comprises a first projection coupled to a first end of the second curved member and a second projection coupled to a second, opposite end of the second curved member.

6. The device of claim 5, wherein the first curved member comprises a first channel at least partially surrounding first projection and a second channel at least partially surrounding the second projection, and wherein the first and second projections are configured to be translated vertically within the first and second channels, respectively, in response to actuation of the actuator.

7. The device of claim 1, wherein the second curved member further comprises a tissue support surface adjacent to the second tissue contact surface.

8. The device of claim 7, wherein the tissue support surface is raised relative to the second tissue contact surface along a longitudinal axis of the device.

9. The device of claim 7, wherein the tissue support surface and the second tissue contact surface form a slot therebetween, and wherein the slot is configured to receive a portion of a cutting element of the cutting assembly therein during excision of the tissue.

10. The device of claim 1, wherein the first curved member is fixed to the body.

11. The device of claim 10, wherein the first curved member is integrally formed with the body.

12. The device of claim 1, wherein the first and second tissue contact surfaces face one another.

13. (canceled)

14. The device of claim 1, wherein the actuator comprises a lever.

15. The device of claim 1, wherein the cutting assembly is coupled to and moveable along the first curved member.

16. (canceled)

17. The device of claim 1, wherein the cutting assembly comprises a housing comprising a handle and a cutting element coupled to the housing.

18. The device of claim 17 further comprising a cutting assembly lock coupled to one or both of the first and second curved members, wherein the cutting assembly lock is configured to prevent movement of the cutting element after the cutting assembly is moved from a first position to a second position along the first curved member.

19. The device of claim 18, wherein the first position is an initial position of the cutting assembly prior to tissue excision and the second position is a final position of the cutting assembly after tissue excision.

20. (canceled)

21. The device of claim 1, wherein the device is configured to be used on an upper eyelid, a lower eyelid, an eyeball, an eyebrow, a cheek, a jaw, an underarm, or a neck of a patient.

22. The device of claim 21, wherein the device is configured to be used on only an upper right eyelid or only an upper left eyelid of the patient.

23. (canceled)

24. The device of claim 1, wherein the tissue comprises a lesion on the patient.

25-109. (canceled)

110. The device of claim 1, wherein the on or both of the first or second curved members is transparent.

111. The device of claim 1, wherein the first curved member comprises one or more markings configured to aid in alignment of the device with the tissue.
